(19) United States
(12) Patent Application Publication
(10) Pub. No.: US 2024/0207299 A1
(43) Pub. Date: Jun. 27, 2024

(54) PYRROLOPYRIMIDINE NUCLEOSIDES FOR TREATING OR PREVENTING A SARS-COV-2 INFECTION

(71) Applicant: Chimerix, Inc., Durham, NC (US)

(72) Inventors: Heidi Muth COLTON, Apex, NC (US); Mohammed Anowrul KABIR, Cary, NC (US); Ernest Randall LANIER, Jr., Chapel Hill, NC (US); Phiroze Behram SETHNA, Durham, NC (US); Roy W. WARE, Raleigh, NC (US)

(73) Assignee: Chimerix, Inc., Durham, NC (US)

(21) Appl. No.: 18/553,144

(22) PCT Filed: Mar. 29, 2022
(86) PCT No.: PCT/US2022/022332

§ 371 Date: Sep. 28, 2023

Related U.S. Application Data
(60) Provisional application No. 63/308,435, filed on Feb. 9, 2022.
(60) Provisional application No. 63/167,292, filed on Mar. 29, 2021.

(51) Int. Cl. A61K 31/7064; A61K 9/00; A61P 31/14

(52) U.S. Cl. CPC A61K 31/7064; A61K 9/0078; A61P 31/14

(57) ABSTRACT

The present disclosure provides methods of treating or preventing a SARS-CoV-2 infection or a SARS-CoV-2-infection associated disease or disorder (e.g. COVID-19) comprising administering to a subject in need thereof an effective amount of a pyrrolopyrimidine nucleoside analog of the Formula I, Formula IA, Formula IB or Formula II and pharmaceutical compositions thereof wherein R_{c} and A are defined herein.

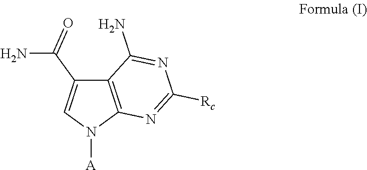

### CROSS-REFERENCE TO RELATED APPLICATIONS

This application claims priority to, and the benefit of, U.S. Provisional Application Nos 63/167,292, filed Mar. 29, 2021, and 63/308,435, filed Feb. 9, 2022, the contents of each of which are incorporated by reference in their entirety.

### BACKGROUND

SARS-CoV-2 and associated conditions (e.g. COVID-19) continues to have serious adverse health effects on individuals across the globe with far reaching and likely long-lasting economic consequences. Accordingly, development of an antiviral effective against SARS-CoV-2 is important to improve the health of infected individuals and as a public health measure to subside the current outbreak, prevent further outbreaks, and treat/prevent further mutations and further outbreaks of SARS-CoV-2 and other coronaviruses.

### SUMMARY

The present disclosure provides methods of treating and/or preventing a SARS-CoV-2 infection and/or a SARS-CoV-2 infection-associated disease or disorder (e.g. COVID-19) with one or more compounds disclosed herein. The disclosure addresses the immediate clinical need for new therapies that can be used to treat and/or prevent SARS-CoV-2-induced disease.

In some aspects, the present disclosure is directed to methods of treating and/or preventing a SARS-CoV-2 infection and/or a SARS-CoV-2 infection-associated disease or disorder (e.g. COVID-19) with one or more compounds of Formula I:

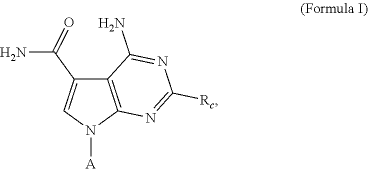

prodrugs thereof, metabolites thereof, and pharmaceutically acceptable salts, solvates, enantiomers, diastereomers, racemates and mixtures thereof, wherein:
A is:

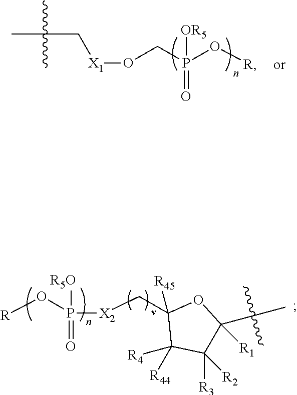

X₁ is CR₁₁R₁₂ or OCH₂CH₂ with the oxygen atom distal to the R moiety in A, in which R₁₁ and R₁₂ are independently hydrogen or substituted or unsubstituted C₁-C₄ alkyl;
X₂ is absent, —O—, —C(O)O—, or —OCH₂— with the oxygen atom distal to the R moiety in A;
each R independently is hydrogen, substituted or unsubstituted C₁-C₆ alkyl,

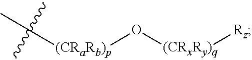

or R is an amino acid residue bound via the carbonyl group of X₂;
v is 0 or 1;
n is 0, 1, 2, or 3 and when X₂ is —C(O)O—, n is 0;
p is 2, 3, 4, 5, 6, 7, 8, 9, 10, or 11;
q is 1, 2, 3, 4, 5, 6, 7, 8, 9, 10, 11, 12, 13, 14, 15, 16, 17, or 18;
R_{z} is hydrogen, halogen, C₁-C₄ alkylthio, C₁-C₄ alkoxy, substituted or unsubstituted C₁-C₄ alkyl, substituted or unsubstituted C₂-C₄ alkenyl, substituted or unsubstituted C₂-C₄ alkynyl, substituted or unsubstituted aryl, substituted or unsubstituted heteroaryl, substituted or unsubstituted cycloalkyl, substituted or unsubstituted cycloalkenyl; or substituted or unsubstituted non-aromatic heterocyclic ring;
Rₐ, R_{b}, Rₓ, and R_{y} are each independently selected from the group consisting of hydrogen, halogen, OH, SH, substituted or unsubstituted C₁-C₆ alkoxy, substituted or unsubstituted aryloxy, substituted or unsubstituted C₁-C₆ alkylthio, substituted or unsubstituted arylthio, substituted or unsubstituted —O-carbonylalkyl, substituted or unsubstituted —O-carbonylaryl, substituted or unsubstituted C₁-C₆ alkyl, substituted or unsubstituted C₂-C₆ alkenyl, substituted or unsubstituted C₂-C₆ alkynyl, substituted or unsubstituted aryl, substituted or unsubstituted heteroaryl, substituted or unsubstituted cycloalkyl, and substituted or unsubstituted cycloaklenyl;
alternatively any Rₐ or R_{b} in (CRₐR_{b})ₚ is taken with another Rₐ or R_{b}, together with the atoms to which they are attached and any intervening atoms therebetween to form a carbon-carbon double or triple bond, a C₆-C₁₀ aryl, 5- to 10-membered heteroaryl, C₃-C₁₀ cycloalkyl, C₄-C₁₀ cycloalkenyl, or 5- to 10-membered non-aromatic heterocyclic ring structure; or any Rₓ or R_{y} in (CRₓR_{y})_{q} is taken with another Rₓ or R_{y}, together with the atoms to which they are attached and any intervening atoms therebetween to form a carbon-carbon double or triple bond, a C₆-C₁₀ aryl, 5- to 10-membered heteroaryl, C₃-C₁₀ cycloalkyl, C₄-C₁₀ cycloalkenyl, or 5- to 10-membered non-aromatic heterocyclic ring structure; or any CRₐR_{b} or CR_{X}R_{y} is replaced by oxygen, sulfur, sulfinyl (SO) or sulfonyl (SO₂);
R₁ and R₄₅ are each independently hydrogen, halogen, substituted or unsubstituted C₁-C₆ alkyl, substituted or unsubstituted C₃-C₆ cycloalkyl, substituted or unsubstituted C₂-C₆ alkenyl, substituted or unsubstituted C₄-C₈ cycloalkenyl, substituted or unsubstituted C₂-C₆ alkynyl, substituted or unsubstituted C₈-C₁₂ cycloalkynyl, azido, —OH, substituted or unsubstituted C₁-C₆ alkoxy, substituted or unsubstituted amino, —SH, or substituted or unsubstituted C₁-C₆ alkylthio;
each of R₂, R₃, R₄ and R₄₄ independently is hydrogen, halogen, substituted or unsubstituted C₁-C₆ alkyl, N₃, OH, substituted or unsubstituted C₁-C₆ alkoxy, substituted or unsubstituted amino, SH, or substituted or unsubstituted C₁-C₆ alkylthio; alternatively R₃ and one of R₄ and R₄₄ together with the atoms to which they are attached form a carbon-carbon double bond;
R₅ is hydrogen, R, M, substituted or unsubstituted aryl, substituted or unsubstituted aralkyl, substituted or unsubstituted C₁-C₆ alkyl, substituted or unsubstituted C₁-C₆ heteroalkyl, substituted or unsubstituted cycloalkyl, substituted or unsubstituted non-aromatic heterocyclic ring, or substituted or unsubstituted heteroaryl; wherein M is a cation; and
R_{c} is substituted or unsubstituted C₁-C₆ alkyl, substituted or unsubstituted C₃-C₆ cycloalkyl, substituted or unsubstituted C₂-C₆ alkenyl, substituted or unsubstituted C₄-C₈ cycloalkenyl, substituted or unsubstituted C₂-C₆ alkynyl, substituted or unsubstituted C₈-C₁₂ cycloalkynyl, or substituted or unsubstituted aryl.

In some aspects, the present disclosure relates to methods of treating and/or preventing a SARS-CoV-2 infection and/or a SARS-CoV-2 infection-associated disease or disorder (e.g. COVID-19) with one or more compounds of Formula IA:

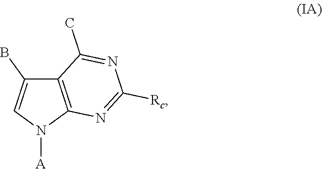

prodrugs thereof, metabolites thereof, and pharmaceutically acceptable salts, solvates, enantiomers, diastereomers, racemates or mixtures thereof, wherein:
A is:

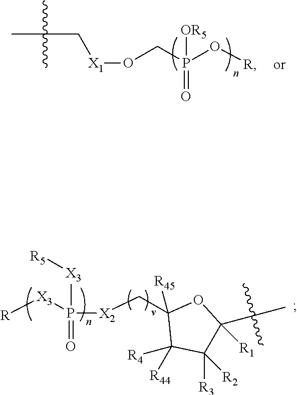

X₁ is —CR₁₁R₁₂— or —OCH₂CH₂— wherein the oxygen atom is distal to the R moiety in A;
R₁₁ and R₁₂ are independently hydrogen or C₁-C₄ alkyl, wherein the alkyl is optionally substituted with one or more halogen, —OH, —SH, or —NH₂;
X₂ is absent, —O—, —C(O)O—, or —OCH₂— wherein the oxygen atom is distal to the R moiety in A;
X₃ is independently —O— or —NH—;
B is independently —C(O)NH₂, aryl, or heteroaryl;
C is independently —OR, —NHR, or —N═CHN(R)₂;
each R independently is hydrogen or —C₁-C₆alkyl, wherein the alkyl is optionally substituted with one or more —OH, —SH, or —NH₂, oxo, Rₐ, or —ORₐ,

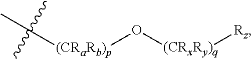

or an amino acid residue bound via the carbonyl group,
v is 0 or 1;
n is 0, 1, 2, or 3 and when X₂ is —C(O)O—, n is 0;
p is 2, 3, 4, 5, 6, 7, 8, 9, 10, or 11;
q is 1, 2, 3, 4, 5, 6, 7, 8, 9, 10, 11, 12, 13, 14, 15, 16, 17, or 18;
R_{z} is hydrogen, halogen, —C₁-C₄ alkylthio, —C₁-C₄ alkoxy, —C₁-C₄alkyl, —C₂-C₄alkenyl, —C₂-C₄alkynyl, aryl, heteroaryl, —C₃-C₈cycloalkyl, —C₄-C₈cycloalkenyl, or 3- to 5-membered nonaromatic heterocycle, wherein each alkylthio, alkoxy, alkyl, alkenyl, alkynyl, aryl, heteroaryl, cycloalkyl, cycloalkenyl, or heterocycle is optionally substituted with one or more halogen, —OH, —SH, or —NH₂;
Rₐ, R_{b}, Rₓ, and R_{y} are each independently selected from the group consisting of hydrogen, halogen, —OH, —SH, —C₁-C₆ alkoxy, aryloxy, —C₁-C₆alkylthio, arylthio, —OC(O)C₁-C₆alkyl, —OC(O)aryl, —C₁-C₆alkyl, —C₂-C₆ alkenyl, —C₂-C₆alkynyl, aryl, heteroaryl, —C₃-C₈cycloalkyl, and —C₄-C₈cycloaklenyl, wherein each alkoxy, aryloxy, alkylthio, arylthio, alkyl, aryl, alkenyl, alkynyl, heteroaryl, cycloalkyl, or cycloalkenyl is optionally substituted with one or more halogen, —OR₁₁, —SR₁₁, or —NR₁₁R₁₂;
or any two Rₐ or R_{b}, together with the atom to which they are both attached, can combine to form a C₃-C₈ spirocycloalkyl or 3- to 8-membered spiroheterocycle;
or any two Rₐ or R_{b}, when on adjacent atoms, can combine to form a cis- or trans-carbon-carbon double bond or a carbon-carbon triple bond;
or any two Rₐ or R_{b}, when on adjacent atoms, can combine to form an oxo, aryl, heteroaryl, —C₃-C₁₀cycloalkyl, —C₄-C₁₀cycloalkenyl, or 5- to 10-membered ring heterocycle;
or any CRₐR_{b} can be replaced by —O—, —S—, —S(O)—, or —SO₂—;
or any two Rₓ or R_{y}, together with the atom to which they are both attached, can combine to form a —C₃-C₈ spirocycloalkyl or 3- to 8-membered spiroheterocycle;
or any two Rₓ or R_{y}, when on adjacent atoms, can combine to form a cis- or trans-carbon-carbon double bond or a carbon-carbon triple bond;
or any two Rₓ or R_{y}, when on adjacent atoms, can combine to form an oxo, aryl, heteroaryl, —C₃-C₁₀cycloalkyl, —C₄-C₁₀cycloalkenyl, or 5- to 10-membered ring heterocycle;
or any CR_{X}R_{y} can be replaced by —O—, —S—, —S(O)—, or —SO₂—;
R₁ and R₄₅ are each independently hydrogen, halogen, —N₃, —OH, —NH₂, —SH, —C₁-C₆alkyl, —C₃-C₆ cycloalkyl, —C₂-C₆ alkenyl, —C₄-C₈ cycloalkenyl, —C₂-C₆ alkynyl, —C₈-C₁₂ cycloalkynyl, —C₁-C₆alkoxy, or —C₁-C₆ alkylthio wherein each alkyl, cycloalkyl, alkenyl, cycloalkenyl, alkynyl, cycloalkynyl, alkoxy or alkylthio is independently substituted with one or more halogen, —N₃, —OH, —NH₂, or —SH;
R₂, R₃, R₄ and R₄₄ are each independently hydrogen, halogen, —N₃, —OH, —NH₂, —SH, —C₁-C₆alkyl, —C₁-C₆alkoxy, or —C₁-C₆alkylthio, wherein each alkyl, alkoxy, or alkylthio is optionally substituted with one or more halogen, oxo, —N₃, —OH, —NH₂, or —SH;
or R₃ and one of R₄ and R₄₄, together with the atoms to which they are attached, can form a carbon-carbon double bond;
or R₃ and one of R₄ and R₄₄, together with the atoms to which they are attached, can combine to form a 4- to 8-membered cycloalkyl or heterocycle optionally substituted with C₁-C₆ alkyl;
R₅ is independently hydrogen, —R, M⁺, aryl, aralkyl, —C₁-C₆ alkyl, —C₁-C₆ heteroalkyl, cycloalkyl, non-aromatic heterocyclic ring, or heteroaryl, wherein M⁺ is a cation and wherein each aryl, aralkyl, alkyl, heteroalkyl, cycloalkyl, heterocycle, or heteroaryl is optionally substituted with one or more halogen, —N₃, —OH, —NH₂, or —SH; and
R¹¹ and R¹² are each independently, at each occurrence, hydrogen, halogen, —OH, —SH, —C₁-C₆ alkoxy, aryloxy, —C₁-C₆alkylthio, arylthio, —OC(O)C₁-C₆alkyl, —OC(O)aryl, —C₁-C₆alkyl, —C₂-C₆ alkenyl, —C₂-C₆alkynyl, aryl, heteroaryl, —C₃-C₈cycloalkyl, and —C₄—C₈cycloaklenyl, wherein each alkyl, aryl, alkenyl, alkynyl, heteroaryl, cycloalky and cycloalkenyl is optionally substituted with one or more halogen, —N₃, —OH, —NH₂, or —SH;
R_{c} is —C₁-C₆ alkyl, —C₃-C₆ cycloalkyl, —C₂-C₆ alkenyl, —C₄-C₈ cycloalkenyl, —C₂-C₆ alkynyl, —C₈-C₁₂ cycloalkynyl, or aryl, wherein each alkyl, cycloalkyl, alkenyl, cycloalkenyl, or aryl is optionally substituted with one or more halogen, —N₃, —OH, —NH₂, or —SH;
   wherein any of the nitrogen atoms in the fused pyrimidine ring can be oxidized.

In some aspects, the present disclosure relates to methods of treating and/or preventing a SARS-CoV-2 infection and/or a SARS-CoV-2 infection-associated disease or disorder (e.g. COVID-19) with one or more compounds of Formula II:

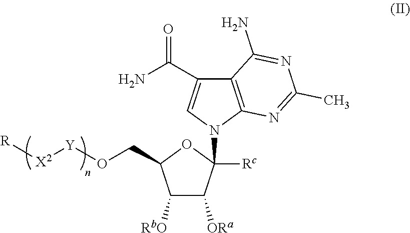

prodrugs thereof, metabolites thereof, and pharmaceutically acceptable salts, solvates, enantiomers, diastereomers, racemates or mixtures thereof, wherein:
Y is —C(O)—, or

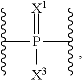

wherein X¹ is independently O, NH, or S, X² is independently a bond, —O—, —S—, or —NH—, and X³ is independently —OR, —NHR, or —SR;
R is independently —H, —C₁-C₂₀alkyl, —C₂-C₂₀alkenyl, —C₂-C₂₀alkynyl, —C₃-C₈cycloalkyl, —C₄-C₈cycloalkenyl, aryl, heteroaryl, or heterocyclyl, wherein each alkyl, alkenyl, alkynyl, cycloalkyl, cycloalkenyl, aryl, heteroaryl, or heterocyclyl is optionally substituted with one or more halogen, oxo, R¹, —OR¹, —NR¹R², —SR¹, —OC(O)R¹, —C(O)OR¹, —NHC(O)OR¹, or —NHC(O)R¹;
Rₐ and R^{b} are each independently, at each occurrence, —H, —C₁-C₂₀alkyl, —C₂-C₂₀alkenyl, —C₂-C₂₀alkynyl, —C₃-C₈cycloalkyl, —C₄-C₈cycloalkenyl, aryl, heteroaryl, or heterocyclyl, wherein each alkyl, alkenyl, alkynyl, cycloalkyl, cycloalkenyl, aryl, heteroaryl, or heterocyclyl is optionally substituted with one or more halogen, oxo —OR¹, —NR¹R², —SR¹, —OC(O)R¹, —C(O)OR¹—NHC(O)OR¹, or —NHC(O)R¹;
R¹ and R² are each independently, at each occurrence, —H, —C₁-C₂₀alkyl, —C₂-C₂₀alkenyl, —C₂-C₂₀alkynyl, —C₃-C₈cycloalkyl, —C₄-C₈cycloalkenyl, aryl, heteroaryl, or heterocyclyl, wherein each alkyl, alkenyl, alkynyl, cycloalkyl, cycloalkenyl, aryl, heteroaryl, or heterocyclyl is optionally substituted with one or more halogen, oxo, R³, R⁴, —OR³, —NR³R₄, —SR³, —OC(O)R³, —C(O)OR³, —NHC(O)OR³, or —NHC(O)R³;
R³ and R⁴ are each independently, at each occurrence, —H, —C₁-C₂₀alkyl, —C₂-C₂₀alkenyl, —C₂-C₂₀alkynyl, —C₃-C₈cycloalkyl, —C₄-C₈cycloalkenyl, aryl, heteroaryl, or heterocyclyl, wherein each alkyl, alkenyl, alkynyl, cycloalkyl, cycloalkenyl, aryl, heteroaryl, or heterocyclyl is optionally substituted with one or more halogen, oxo, aryl, heteroaryl, —OH, —NH₂, —SH, —OC(O)H, —C(O)OH, —NHC(O)OH, or —NHC(O)H;
R^{c} is independently —H or -D; and
n is independently 0, 1, 2 or 3.

In another aspect, the present disclosure is directed to methods of treating and/or preventing a SARS-CoV-2 infection and/or a SARS-CoV-2 infection-associated disease or disorder (e.g. COVID-19) comprising administering to a subject in need thereof an effective amount of a compound described herein (e.g., a compound of Formula I, Formula IA, Formula IB or Formula II, or a prodrug thereof). In some aspects, the present disclosure is directed to methods for treating or preventing a SARS-CoV-2 infection or a SARS-CoV-2-infection associated disease or disorder (e.g. COVID-19), comprising administering to a subject in need thereof an effective amount of a compound of Formula II or a pharmaceutically acceptable salt, solvate, enantiomer, diastereomer, racemate or mixture thereof. In some aspects, the present disclosure is directed to methods for treating or preventing a SARS-CoV-2 infection or a SARS-CoV-2-infection associated disease or disorder (e.g. COVID-19), comprising administering to a subject in need thereof an effective amount of a compound of Formula II or a pharmaceutically acceptable salt or solvate thereof.

In some aspects, the present disclosure is directed to methods of treating and/or preventing a SARS-CoV-2 infection and/or a SARS-CoV-2 infection-associated disease or disorder (e.g. COVID-19) with a pharmaceutical composition comprising a compound of Formula I, Formula IA, Formula IB or Formula II, or a prodrug thereof, and a pharmaceutically acceptable carrier. The present disclosure also relates to use of the pharmaceutical formulation disclosed herein in the manufacture of a medicament for treating and/or preventing a SARS-CoV-2 infection and/or a SARS-CoV-2 infection-associated disease or disorder (e.g. COVID-19).

In some aspects, the present disclosure is directed to the use of a compound of the present disclosure for preparing a medicament for preventing a SARS-CoV-2 infection or a SARS-CoV-2-infection associated disease or disorder (e.g. COVID-19) in a patient in need thereof.

In some aspects, the present disclosure is directed to compounds for use preventing a SARS-CoV-2 infection or a SARS-CoV-2-infection associated disease or disorder (e.g. COVID-19) in a patient in need thereof.

In some aspects, the present disclosure is directed to methods for treating or preventing a SARS-CoV-2 infection or a SARS-CoV-2-infection associated disease or disorder (e.g. COVID-19) in a patient in need thereof, comprising administering a compound described herein, a prodrug thereof, or a pharmaceutically acceptable salt, solvate, enantiomer, diastereomer, racemate or mixture thereof, wherein said administration results in the in vivo formation of one or more active metabolites. Unless otherwise defined, all technical and scientific terms used herein have the same meaning as commonly understood by one of ordinary skill in the art to which this disclosure belongs. In the case of conflict, the present specification, including definitions, will control. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents, and other references mentioned herein are incorporated by reference. The references cited herein are not admitted to be prior art to the claimed disclosure. In addition, the materials, methods, and examples are illustrative only and are not intended to be limiting.

Other features and advantages of the present disclosure will be apparent from the following detailed description and claims.

### BRIEF DESCRIPTION OF THE DRAWINGS

FIGS. 1A and 1B show dose response curves for Compound 1 against MHV nLuc and SARS-CoV-2-nLuc. Results are relative to control cells treated with DMSO. FIG. 1A: Dose response curve for Compound 1 against murine hepatitis virus (MHV) encoding an nLuc reporter in DBT cells. Antiviral activity (black line) and cell viability (white line) is shown for each concentration. FIG. 2B: A representative dose response curve for Compound 1 against SARS-CoV-2 encoding an nLuc reporter in primary HAE cells grown at the air-liquid interface. Antiviral activity (black line) is shown for each concentration. Remdesivir (RDV) was included as a positive control.

FIG. 2 is a Coronavirus Phylogenetic Tree Showing Relationship Between SARS and Bat-Derived Coronaviruses WIV1 and SHC014. Figure is modified from Holbrook M G, Anthony S J, Navarrete-Macias I, et al. Updated and Validated Pan-Coronavirus PCR Assay to Detect All Coronavirus Genera. Viruses. 2021. 13:599. Shaded arrows indicate SARS-CoV-2 and bat-derived SAR-like viruses. Empty arrow indicates MHV (murine CoV).

FIG. 3 shows Cq values for the SARS-CoV-2 genome detection assay described in Example 4. Cq=quantification cycle.

FIGS. 4A-4F show the effects of pre- and post-infection administration of Compound 1 on the clinical signs of Covid-19 disease on BALB/c mice. HPI=hours post infection.

FIG. 5 shows the effects of pre- and post-infection administration of Compound 1 on the body weight of BALB/c mice infected with SARS-CoV-2-MA10. HPI=hours post infection.

FIG. 6 shows the effects of pre- and post-infection administration of Compound 1 on the gross lung discoloration of BALB/c mice infected with SARS-CoV-2-MA10. HPI=hours post infection.

FIGS. 7A and 7D show postcaval lung lobe nsp4 qPCR data in BALB/c mice infected with SARS-CoV-2-MA10 and treated with Compound 1 pre- and post-infection compared to vehicle. FIGS. 7B and 7E show nasal viral titer data in BALB/c mice infected with SARS-CoV-2-MA10 and treated with Compound 1 pre- and post-infection compared to vehicle. For FIG. 7B, “*” indicates p<0.005. FIG. 7C shows lung viral titer data (Combined superior and middle lung lobes) in BALB/c mice infected with SARS-CoV-2-MA10 and treated with Compound 1 pre- and post-infection compared to vehicle. HPI=hours post infection. ns=not significant. nsp4 is a membrane-spanning coronavirus nonstructural protein.

FIGS. 8A and 8B show postcaval lung lobe nsp4 qPCR data and lung viral titer data in BALB/c mice treated prophylactically with Compound 1 8 hours prior to infection with SARS-CoV-2-MA10.

### DETAILED DESCRIPTION

This application relates to methods of treating and/or preventing a SARS-CoV-2 infection using pyrrolopyrimidine nucleoside analogs, phospholipid conjugates thereof, prodrugs thereof, and morphic forms thereof. This application also related to methods of treating and/or preventing a SARS-CoV-2 infection using pharmaceutical compositions comprising pyrrolopyrimidine nucleoside analogs, phospholipid conjugates thereof, and morphic forms thereof.

The present disclosure provides methods for treating or preventing a SARS-CoV-2 infection or SARS-CoV-2 infection associated disease or disorder (e.g. COVID-19) with a nucleoside compound, a phosphonate analog thereof, a metabolite thereof, or a prodrug thereof.

Nucleoside phosphonates represent a target class of antivirals to inhibit viruses which rely on viral encoded enzymes using ribonucleotides or deoxyribonucleotides as substrates. However, without wishing to be bound by theory, one block to efficacy for this class of antivirals is the requirement for biochemical modification of the administered agent inside target cells to form the active antiviral nucleoside triphosphate. In some embodiments, if a nucleoside is delivered, three phosphorylation steps are required to form the triphosphate. Delivery of nucleoside phosphonates effectively bypasses the first phosphorylation, but can exacerbate problems of delivering clinically useful amounts of the charged drug across the lipid bilayers surrounding cells.

Without wishing to be bound by theory, lipid conjugation can be used to disguise oral drugs, including nucleoside phosphonates and phosphates, as natural compounds that are readily absorbed by the body. Specifically, in some embodiments, nucleoside phosphonates can be modified to resemble partially metabolized (monoacyl) phospholipids. In some embodiments, in contrast to normal diacylphospholipids, monoacyl lipid-modified nucleosides can readily penetrate the enterocytes lining the lumen of the gut, enter the circulating blood and/or lymph and, unlike standard drugs, remain intact. Consequently, the lipid moiety can do more than deliver the nucleoside to the plasma; it can facilitate efficient uptake into the target cells. The lipid can be cleaved in the cytoplasmic compartment of the target cells and in the case of nucleoside analog conjugates, can yield the corresponding monophosphate. Overall, this strategy can lead to greatly increased levels of the active antiviral at the site of viral replication.

It has been reported that metabolism of nucleoside analogs to the active triphosphate forms may be influenced by the different cell lines used in the assays. This has been demonstrated for the guanosine analog AT-527 in MRC5 cells where poor phosphorylation was noted along with lack of antiviral activity against SARS-CoV-2. A similar observation was made in Vero cells for the same compound (Good, Steven S et al. “AT-527, a Double Prodrug of a Guanosine Nucleotide Analog, Is a Potent Inhibitor of SARS-CoV-2 In Vitro and a Promising Oral Antiviral for Treatment of COVID-19.” Antimicrobial agents and chemotherapy vol. 65,4 e02479-20. 18 Mar. 2021). In the case of remdesivir, a similar finding was noted where the potency was much lower in Vero cells compared to other cells (Tao, Sijia et al. “Comparison of anti-SARS-CoV-2 activity and intracellular metabolism of remdesivir and its parent nucleoside.” Current research in pharmacology and drug discovery vol. 2 (2021): 100045).

In some embodiments, the compounds of the present disclosure have improved efficacy/toxicity ratio compared to compounds in the art used similarly. In some embodiments, Compound 1 exhibits improved efficacy/toxicity compared to compounds in the art used similarly. In some embodiments, the improved efficacy/toxicity ratio arises from increased efficacy. In some embodiments, the improved efficacy/toxicity ratio arises from decreased toxicity compared to compounds in the art used similarly. In some embodiments, the improved efficacy/toxicity ratio arises from both increased efficacy and decreased toxicity compared to compounds in the art used similarly. Without wishing to be bound by theory, it may be that inhaled deliver of a compound described herein (e.g., a compound of Formula I, Formula IA, Formula IB or Formula II, or a prodrug thereof) concentrates exposure at the site of viral replication, thereby reducing systemic exposure.

### Definitions

Certain compounds of the present disclosure and definitions of specific functional groups are also described in more detail below.

It will be appreciated that the compounds, as described herein, may be substituted with any number of substituents or functional moieties. In general, the term “substituted” whether preceded by the term “optionally” or not, and substituents contained in formulas disclosed herein, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this disclosure, heteroatoms such as nitrogen may have hydrogen substituents and/or any permissible substituents of organic compounds described herein which satisfy the valencies of the heteroatoms. The nitrogen and sulfur heteroatoms may optionally be oxidized, and the nitrogen heteroatom may optionally be quaternized. Examples of substituents on the moieties disclosed herein (e.g., alkyl, alkenyl, alkynyl, alkoxy, aryl, heteroaryl, cycloalkyl, cycloalkenyl, non-aromatic heterocycle groups) include, but are not limited to, alkenyl, alkynyl, halogen, haloalkyl, alkoxy, alkylthio, alkylsulfinyl, alkylsulfonyl, heteroaryl, aryl, cycloalkyl, cycloalkenyl, non-aromatic heterocycle, hydroxyl, carbamoyl, oxo, amino, nitro, azido, —SH, and —CN.

The present disclosure is intended to include all isotopes of atoms occurring in the present compounds. Isotopes include those atoms having the same atomic number but different mass numbers. In particular one, some, or all hydrogens may be deuterium. Radioactive isotopes may be used, for instance for structural analysis or to facilitate tracing the fate of the compounds or their metabolic products after administration. By way of general example and without limitation, isotopes of hydrogen include deuterium and tritium and isotopes of carbon include C-13 and C-14. For example, compounds of Formula I include those wherein R₁ is H or D; R₂ and R₃ are independently H, D, OH, OD, CH₃, or CD₃; and/or R₄ is H, D, CH₃, or CD₃.

The term “independently” is used herein to indicate that the variable, such as atom or functional group, which is independently applied, varies independently from application to application. For example, where more than one substituent or atom (carbon or heteroatom, such as oxygen (O), sulfur (S), or nitrogen (N)) occurs, each substituent or atom is independent of another substituent or atom and such substituents or atom can also alternate.

The term “alkyl”, as used herein, refers to saturated, straight-chain or branched hydrocarbon radicals containing, in certain embodiments, between one and twenty, including between one and ten, or between one and six, carbon atoms. Branched means that one or more lower C₁-C₆ alkyl groups such as methyl, ethyl or propyl are attached to a linear alkyl chain. Exemplary alkyl groups include methyl, ethyl, n-propyl, i-propyl, n-butyl, t-butyl, n-pentyl, and 3-pentyl. Examples of C₁-C₆ alkyl radicals include, but are not limited to, methyl, ethyl, propyl, isopropyl, butyl, tert-butyl, neopentyl, n-hexyl radicals; and examples of C₁-C₈ alkyl radicals include, but are not limited to, methyl, ethyl, propyl, isopropyl, n-butyl, tert-butyl, neopentyl, n-hexyl, heptyl, octyl radicals. Examples of C₁-C₂₀ alkyl radicals include but are not limited to hexadecamethyl, hexadecaethyl, hexadecopropyl, octadecamethyl, octadecaethyl, octadecapropyl and the like.

The term “alkenyl”, as used herein, denotes a monovalent straight or branched group derived from a hydrocarbon moiety containing, in certain embodiments, from two to six, or two to eight, or two to twenty carbon atoms having at least one carbon-carbon double bond. The double bond may or may not be the point of attachment to another group. Examples of C₂-C₈ alkenyl groups include, but are not limited to, for example, ethenyl, propenyl, butenyl, 1-methyl-2-buten-1-yl, heptenyl, octenyl and the like. As defined herein, “akenyl” groups include both cis- and trans-isomers.

The term “alkynyl”, as used herein, denotes a monovalent straight or branched group derived from a hydrocarbon moiety containing, in certain embodiments, from two to six, or two to eight, or two to twenty carbon atoms having at least one carbon-carbon triple bond. The triple bond may or may not be the point of attachment to another group. Examples of C₂-C₈ alkynyl groups include, but are not limited to, for example, ethynyl, propynyl, butynyl and the like.

The term “alkoxy” refers to an —O-alkyl radical.

The term “thioalkyl” or “alkylthio” refers to an —S-alkyl radical. In some embodiments, thio group can be replaced by a sulfinyl (SO) or sulfonyl (SO₂).

The terms “hal”, “halo”, or “halogen”, as used herein, refer to an atom selected from fluorine, chlorine, bromine and iodine.

The terms “haloalkyl”, “haloalkenyl”, or “haloalkynyl”, as used herein refer to an alkyl, alkenyl or alkynyl that is substituted with one or more halogens or halo groups. Examples of haloalkyl include but are not limited to CF₃, CH₂CF₃, CCl₃.

The term “aryl”, as used herein, refers to a mono- or poly-cyclic carbocyclic ring system having one or more aromatic rings, fused or non-fused, including, but not limited to, phenyl, naphthyl, tetrahydronaphthyl, indanyl, idenyl and the like. The term aryl includes indoline.

The term “cycloalkyl”, as used herein, denotes a monovalent group derived from a monocyclic or polycyclic saturated carbocyclic ring compound. Examples of C₃-C₈-cycloalkyl (3- to 8-membered cycloalkyl) include, but not limited to, cyclopropyl, cyclobutyl, cyclopentyl, cyclohexyl, cyclopentyl and cyclooctyl; and examples of C₃-C₁₂-cycloalkyl include, but not limited to, cyclopropyl, cyclobutyl, cyclopentyl, cyclohexyl, bicyclo [2.2.1]heptyl, and bicyclo [2.2.2] octyl and the like.

The term “cycloalkenyl”, as used herein, denotes a monovalent group derived from a monocyclic or polycyclic partially unsatured (i.e., non-aromatic) carbocyclic ring compound. In other words, it refers to a monovalent group derived from a monocyclic or polycyclic carbocyclic ring compound having at least one carbon-carbon double bond. Examples of such groups include, but are not limited to, cyclopropenyl, cyclobutenyl, cyclopentenyl, cyclohexenyl, cycloheptenyl, cyclooctenyl, and the like.

The term “cycloalkynyl,” as used herein, denotes a monovalent group derived from a monocyclic or polycyclic partially unsaturated (i.e., non-aromatic) carbocyclic compound having at least one carbon-carbon triple bond. Examples include cyclooctyne.

The term “heteroaryl”, as used herein, refers to a mono- or poly-cyclic (e.g., bi-, or tri-cyclic or more) fused or non-fused, radical or ring system having at least one aromatic ring, having from five to ten ring atoms of which at least one ring atom is selected from S, O, P, and N. In other words, heteroaryl is aryl where containing at least one heteroatom. Examples of heteroaryl include but are not limited to pyridinyl, furanyl, thiazolyl, imidazolyl, indolyl, benzofuranyl, and the like.

The term “5- or 6-membered heteroaryl”, is taken to mean a ring having five to twelve ring atoms of which at least one ring atom is selected from S, O, P, and N. Heteroaryl includes, but is not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl, and the like.

The term “non-aromatic heterocyclic” ring or “non-aromatic heterocycle,” as used herein, refers to a saturated or unsaturated, non-aromatic monocyclic or polycyclic, fused or non-fused system, where, for example, at least one ring contains between one and four heteroatoms independently selected from oxygen, sulfur, phosphorous and nitrogen. The nitrogen and sulfur heteroatoms may optionally be oxidized, and the nitrogen heteroatom may optionally be quaternized. Representative non-aromatic heterocyclic groups include, but are not limited to, [1,3]dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

As used herein, the term “oxo” is understood to describe a carbonyl group (i.e., C(O)).

As described herein, compounds of the disclosure may optionally be substituted with one or more substituents, such as those illustrated generally above, or as exemplified by particular classes, subclasses, and species of the disclosure. It will be appreciated that the phrase “optionally substituted” is used interchangeably with the phrase “substituted or unsubstituted.” In general, the term “substituted,” whether preceded by the term “optionally” or not, refers to the replacement of hydrogen radicals in a given structure with the radical of a specified substituent. Unless otherwise indicated, an optionally substituted group may have a substituent at each substitutable position of the group, and when more than one position in any given structure may be substituted with more than one substituents selected from a specified group, the substituent may be either the same or different at every position. The term “protected” as described herein, refers to functional groups or compounds of the present disclosure having a protecting group used in synthesis to temporarily mask the characteristic chemistry of a functional group (such as hydroxyl, amino, carboxyl, etc.) because it interferes with another reaction. After completion of the reaction, these protecting groups are removed by common methods, or protected compounds are used as prodrugs or as the compounds of the disclosure.

The term “prodrug” or “pharmaceutically acceptable prodrugs”, as used herein refers to compounds that are rapidly transformed in vivo to yield the parent compound, for example by hydrolysis or enzymatic reaction. Examples of prodrugs include esters and other pharmaceutically acceptable derivatives, which, upon administration to a subject, are capable of providing active compounds of this disclosure. For example, where the metabolite is formed in an enzymatic reaction in which the molecular weight of the compound increases, contemplated enzymes include kinases (e.g., formation of a mono-, di-, or triphosphate from contemplated compounds), transferases (e.g., terminal nucleotidyl transferase adds a polynucleotide to the compound), or polymerases (e.g., DNA-dependent RNA polymerase adds a polyribonucleotide to the compound). Similarly, where the reaction is a non-enzymatic reaction, the molecular weight of contemplated compounds may be increased via thiol oxidation to form the corresponding disulfide (e.g., where contemplated compounds include an SH group). In another example, the metabolite may be formed in an enzymatic reaction in which the molecular weight of the compound decreases, and contemplated enzymes include aminohydrolases (e.g., deprotection of a protected amino group), phosphatases (removal of a phosphate group from a mono-, di-, or triphosphate), nucleosidases (e.g., hydrolytic cleavage of the heterocyclic base from the sugar), and so on. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce an active compound. The compounds of this invention possess antiviral activity against SARS-CoV-2, or are metabolized to a compound that exhibits such activity, or both. For example, prodrugs of Compound 1 include, but are not limited to, one or more compounds described herein. Further, prodrugs of Compound 1 include, but are not limited to, derivatives of Compound 1 having modification at one or more the places marked below.

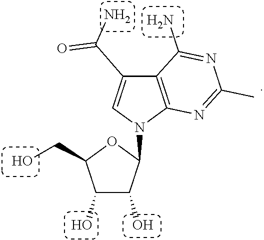 [SMILES: Cc1nc(N)c2c(C(N)=O)cn([C@@H]3O[C@H](CO)[C@@H](O)[C@H]3O)c2n1]

A skilled artisan would appreciate the scope of the modification with the common knowledge of developing prodrugs based on the structure of Compound 1. Examples of strategies and options for preparing such prodrugs can be found in Stella (Prodrugs as Therapeutics, Expert Opinion on Therapeutic Patents, 14:3, 277-280 (2004); incorporated by reference), Rautio (Prodrugs and Targeted Delivery, Wiley-VCH Verlag GmbH & Co. KGaA, 2011; incorporated by reference), and Stella et al. (Prodrugs: Challenges and Rewards, Vol. 1-2, Springer & AAPS Press, 2007; incorporated by reference).

While the compounds of the present disclosure may be prepared using conventional synthetic approaches, it should be especially recognized that the compounds according to the present disclosure also include their metabolites.

The term “metabolite”, as used herein, refers to a metabolite of a referenced compound that could be formed inside or outside of a cell in vitro or in vivo, and wherein the metabolite may be formed in an enzymatic reaction or a non-enzymatic reaction. Especially contemplated metabolites will include active metabolites, wherein the term “active metabolite” as used herein refers to any metabolite of contemplated compounds that exhibits biological activity (e.g., antiviral activity). It should be recognized that biological activity may be readily determined in an assay in which the metabolite is formed in sufficient quantity (e.g., in human, animals, or cell culture) after administration of contemplated compounds to the assay. As seen in PCT/US2016/046056, Example 4, Compound 1 converts to a triphosphate in vitro. Specifically, when cells were incubated with Compound 1, the corresponding triphosphate (i.e., Compound 1-TP) was produced. The level of Compound 1-TP was 12 to 23-fold higher than Compound 1 after the incubation period. In some embodiments, the active metabolite is a monophosphate, a diphosphate, or a triphosphate analogue of a compound described herein (e.g. a compound of Formula I, Formula IA, Formula IB or Formula II appended to one, two, or three phosphate groups).

It is understood that a treatment “with” a compound intends to encompass the treatment comprising administering, or causing to be administered (e.g., by administering a prodrug), the referenced compound. For example, the treatment with a prodrug of Compound 1 encompasses the treatment comprising administering the prodrug. For another example, the treatment with Compound 1 encompasses the treatment comprising administering Compound 1, or causing to be administered Compound 1 (e.g., by administering a prodrug of Compound 1 which, e.g., converts to Compound 1 through metabolism). For another example, the treatment with a metabolite of Compound 1 encompasses the treatment comprising causing to be administered the metabolite of Compound 1 (e.g., by administering or causing to be administered Compound 1 or a prodrug thereof, which, e.g., converts to the metabolite of Compound 1 through metabolism).

The term “administer” or “administering”, as used herein, refers to delivering into the referred subject. In some embodiments, the subject is human, and the compound is administered into, e.g., the body of the human. In some embodiments, the compound (e.g., a prodrug) is administered by way of a pharmaceutical formulation or a pharmaceutical composition thereof.

The term “cause to be administered” or “causing to be administered”, as used herein, refers to causing to be delivered into the referred subject. In some embodiments, the subject is human, and the compound is causing to be administered into, e.g., the body of the human. In some embodiments, the compound (e.g., a drug) is caused to be delivered to the subject by way of administering and metabolizing of a prodrug thereof. In some embodiments, the compound (e.g., a metabolite of drug) is caused to be delivered to the subject by way of administering and metabolizing of the drug or a prodrug thereof.

The term “pharmaceutical” or “pharmaceutically acceptable” when used herein as an adjective, means substantially non-toxic and substantially non-deleterious to the recipient. As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

By “pharmaceutical formulation” it is further meant that the carrier, solvent, excipient(s) and salt must be compatible with the active ingredient of the formulation (e.g. a compound of the disclosure). It is understood by those of ordinary skill in this art that the terms “pharmaceutical formulation” and “pharmaceutical composition” are generally interchangeable, and they are so used for the purposes of this application and include preparations suitable for administration to mammals, e.g., humans.

A “pharmaceutical composition” as used herein relates to a formulation containing a compound of the present disclosure in a form suitable for administration to a subject. In some embodiments, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. As used herein, “pharmaceutically acceptable carrier” may include any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences discloses various carriers used in formulating pharmaceutical compositions and known techniques for the preparation thereof. Except insofar as any conventional carrier medium is incompatible with the compounds such as by producing any undesirable biological effect or otherwise interacting in a deleterious manner with any other component(s) of the pharmaceutical composition, its use is contemplated to be within the scope of this disclosure. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogen free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. “Pharmaceutically acceptable excipient or carrier” also relates to an excipient or carrier that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

The compounds disclosed herein include the compounds themselves, as well as their salts, their solvates, and their prodrugs, if applicable. A salt, for example, can be formed between an anion and a positively charged group (e.g., protonated amino) on a compound of this disclosure. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluroacetate). The term “pharmaceutically acceptable anion” refers to an anion suitable for forming a pharmaceutically acceptable salt. Likewise, a salt can also be formed between a cation and a negatively charged group (e.g., carboxylate) on a compound of this disclosure. Suitable cations include sodium ion, potassium ion, magnesium ion, calcium ion, and an ammonium cation such as tetramethylammonium ion. The compounds of this disclosure also include those salts containing quaternary nitrogen atoms. Examples of prodrugs include esters and other pharmaceutically acceptable derivatives, which, upon administration to a subject, are capable of providing active compounds of this disclosure.

Additionally, physiologically acceptable, i.e. pharmaceutically compatible, salts can be salts of the compounds disclosed herein with inorganic or organic acids. Preference is given to salts with inorganic acids, such as, for example, hydrochloric acid, hydrobromic acid, phosphoric acid or sulphuric acid, or to salts with organic carboxylic or sulphonic acids, such as, for example, acetic acid, trifluoroacetic acid, propionic acid, maleic acid, fumaric acid, malic acid, citric acid, tartaric acid, lactic acid, benzoic acid, or methanesulphonic acid, ethanesulphonic acid, benzenesulphonic acid, toluenesulphonic acid or naphthalenedisulphonic acid.

Other pharmaceutically compatible salts which may be mentioned are salts with customary bases, such as, for example, alkali metal salts (for example sodium or potassium salts), alkaline earth metal salts (for example calcium or magnesium salts) or ammonium salts, derived from ammonia or organic amines, such as, for example, diethylamine, triethylamine, ethyldiisopropylamine, procaine, dibenzylamine, N-methylmorpholine, dihydroabietylamine or methylpiperidine.

As used herein, “pharmaceutically acceptable salts” can refer to derivatives of the compounds of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts can include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, or an alkaline earth metal ion, e.g., an aluminum ion; or coordinates with an organic base such as ethanolamine, diethanolamine, triethanolamine, tromethamine, N-methylglucamine, diethylamine, diethylaminoethanol, ethylenediamine, imidazole, lysine, arginine, morpholine, 2-hydroxyethylmorpholine, dibenzylethylenediamine, trimethylamine, piperidine, pyrrolidine, benzylamine, tetramethylammonium hydroxide and the like.

It should be understood that all references to pharmaceutically acceptable salts include solvent addition forms (solvates) or crystal forms (polymorphs) as defined herein, of the same salt.

The compounds of the present disclosure can also be prepared as prodrugs. In certain embodiments, one or more compounds of the present disclosure are formulated as a prodrug. In certain embodiments, upon in vivo administration, a prodrug is chemically converted to the biologically, pharmaceutically or therapeutically more active form. In certain embodiments, prodrugs are useful because they are easier to administer than the corresponding active form. For example, in certain instances, a prodrug may be more bioavailable (e.g., through oral administration) than is the corresponding active form. In certain instances, a prodrug may have improved solubility compared to the corresponding active form. In certain embodiments, prodrugs are less water soluble than the corresponding active form. In certain instances, such prodrugs possess superior transmittal across cell membranes, where water solubility is detrimental to mobility. In certain embodiments, a prodrug is an ester. In certain such embodiments, the ester is metabolically hydrolyzed to carboxylic acid upon administration. In certain instances the carboxylic acid containing compound is the corresponding active form. In certain embodiments, a prodrug comprises a short peptide (polyaminoacid) bound to an acid group. In certain of such embodiments, the peptide is cleaved upon administration to form the corresponding active form.

In certain embodiments, a prodrug is produced by modifying a pharmaceutically active compound such that the active compound will be regenerated upon in vivo administration. The prodrug can be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound.

Additionally, the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

Some of the compounds of the present disclosure may exist in unsolvated as well as solvated forms such as, for example, hydrates.

“Solvate” means a solvent addition form that contains either a stoichiometric or non-stoichiometric amounts of solvent. Some compounds can have a tendency to trap a fixed molar ratio of solvent molecules in the crystalline solid state, thus forming a solvate. If the solvent is water the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate. In the hydrates, the water molecules are attached through secondary valencies by intermolecular forces, in particular hydrogen bridges. Solid hydrates contain water as so-called crystal water in stoichiometric ratios, where the water molecules do not have to be equivalent with respect to their binding state. Examples of hydrates are sesquihydrates, monohydrates, dihydrates or trihydrates. Equally suitable are the hydrates of salts of the compounds of the disclosure.

The disclosure also includes metabolites of the compounds described herein. Metabolites from chemical compounds, whether inherent or pharmaceutical, are formed as part of the natural biochemical process of degrading and eliminating the compounds. The rate of degradation of a compound is an important determinant of the duration and intensity of its action. Profiling metabolites of pharmaceutical compounds, drug metabolism, is an important part of drug discovery, leading to an understanding of any undesirable side effects.

As used herein, the term “treat,” “treating,” or “treatment” means decreasing the signs, symptoms, markers, and/or any negative effects of a condition in any appreciable degree in a patient who currently has the condition. In some embodiments, treatment may be administered to a subject who exhibits only early signs of the condition for the purpose of decreasing the risk of developing the disease, disorder, and/or condition.

As used herein, the term “prevent,” “prevention,” or “preventing” refers to any method to partially or completely prevent or delay the onset of one or more signs, symptoms or features of a disease, disorder, and/or condition. Prevention treatment may be administered to a subject who does not exhibit signs of a disease, disorder, and/or condition.

The term “therapeutically effective amount”, as used herein, refers to an amount of a pharmaceutical agent to treat, ameliorate, or prevent an identified disease or condition, or to exhibit a detectable therapeutic or inhibitory effect. The effect can be detected by any assay method known in the art. As used herein, “therapeutically effective amount” can also mean that amount necessary to make a clinically observed improvement in the patient. In some embodiments, the composition is formulated such that it comprises an amount that would not cause one or more unwanted side effects. An effective amount of a pharmaceutical agent can also mean that which provides an objectively identifiable improvement as noted by the clinician or other qualified observer. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

As used herein, “subject” means a human or animal (in the case of an animal, more typically a mammal). In some aspects, the subject is a human. In some aspects, the subject is a male. In some aspects, the subject is a female. As used herein, the term “patient” refers to a human subject in a clinical setting.

The compounds of the present disclosure can also be prepared as esters, for example, pharmaceutically acceptable esters. For example, a carboxylic acid function group in a compound can be converted to its corresponding ester, e.g., a methyl, ethyl or other ester. Also, an alcohol or hydroxyl group in a compound can be converted to its corresponding ester, e.g., acetate, propionate, or other esters.

The present disclosure includes new compounds generally represented by Formula I, Formula IA, Formula IB or Formula II, or pharmaceutically acceptable salts thereof, and methods for preparation and uses thereof.

Throughout the description, where compositions are described as having, including, or comprising specific components, it is contemplated that compositions also consist essentially of, or consist of, the recited components.

### Exemplary Administration Routes, Compositions, and Delivery Devices

In some embodiments, the compound of Formula I, Formula IA, Formula IB or Formula II (e.g., Compound 1) or a prodrug thereof, a metabolite thereof, or a pharmaceutically acceptable salt, solvate, enantiomer, diastereomer, racemate or mixture thereof is administered to the subject via parenteral administration.

In some embodiments, the compound of Formula I, Formula IA, Formula IB or Formula II (e.g., Compound 1) or a prodrug thereof, a metabolite thereof, or a pharmaceutically acceptable salt, solvate, enantiomer, diastereomer, racemate or mixture thereof is administered to the subject via inhalation.

In some embodiments, the compound of Formula I, Formula IA, Formula IB or Formula II (e.g., Compound 1) or a prodrug thereof, a metabolite thereof, or a pharmaceutically acceptable salt, solvate, enantiomer, diastereomer, racemate or mixture thereof is administered to the subject via nasal inhalation.

In some embodiments, the compound of Formula I, Formula IA, Formula IB or Formula II (e.g., Compound 1) or a prodrug thereof, a metabolite thereof, or a pharmaceutically acceptable salt, solvate, enantiomer, diastereomer, racemate or mixture thereof is administered to the subject via mouth inhalation.

In some embodiments, the compound of Formula I, Formula IA, Formula IB or Formula II (e.g., Compound 1) or a prodrug thereof, a metabolite thereof, or a pharmaceutically acceptable salt, solvate, enantiomer, diastereomer, racemate or mixture thereof is administered to the subject via pulmonary administration.

In some embodiments, the compound of Formula I, Formula IA, Formula IB or Formula II (e.g., Compound 1) a prodrug thereof, a metabolite thereof, or a pharmaceutically acceptable salt, solvate, enantiomer, diastereomer, racemate or mixture thereof is administered to the lung and/or the respiratory tract of the subject.

As provided herein, the present disclosure provides compositions, systems and methods for the treatment of coronaviral infections. The treatment methods comprise, in one embodiment, delivery of one of the compositions described herein to the lungs of a patient in need thereof, for example, a patient infected with a coronavirus (e.g., SARS-CoV-2). In some embodiments, the coronavirus is a betacoronavirus. In some embodiments, the coronavirus is a Sarbecovirus. In some embodiments, the coronavirus is a severe acute respiratory syndrome-related coronavirus.

The compositions of the present disclosure may be used in any dosage dispensing device adapted for pulmonary administration. Accordingly, in one aspect, the present disclosure provides systems comprising one or more of the compositions described herein and an inhalation delivery device. The device, in one embodiment, is constructed to ascertain optimum metering accuracy and compatibility of its constructive elements, such as container, valve and actuator with the composition and could be based on a mechanical pump system, e.g., that of a metered-dose nebulizer, dry powder inhaler, a propellant-driven metered dose inhalers (pMDI) metered dose inhaler (MDI), soft mist inhaler, or a nebulizer. For example, pulmonary delivery devices include a jet nebulizer, electronic nebulizer, a soft mist inhaler, and a capsule-based dry powder inhaler, all of which are amenable for use with the compositions of the present disclosure.

In some embodiments, the compositions of the disclosure for pulmonary/inhalation/nasal/intranasal administration are formulated in the form of a dry powder formulation, a suspension formulation, a nanosuspension formulation, a microsuspension formulation, or a nebulized spray.

In certain embodiments, the compositions of the disclosure formulated for pulmonary/inhalation/nasal/intranasal administration comprise a propellant, e.g., a hydrocarbon propellant.

The composition, in one embodiment, is administered via a nebulizer, which provides an aerosol mist of the composition for delivery to the lungs of a subject. A nebulizer type inhalation delivery device can contain the compositions of the present disclosure as an aqueous solution or a suspension. In generating the nebulized spray of the compositions for inhalation, the nebulizer type delivery device may be driven ultrasonically, by compressed air, by other gases, electronically or mechanically. The ultrasonic nebulizer device usually works by imposing a rapidly oscillating waveform onto the liquid film of the composition via an electrochemical vibrating surface. At a given amplitude the waveform becomes unstable, whereby it disintegrates the liquids film, and it produces small droplets of the composition. The nebulizer device driven by air or other gases operates on the basis that a high pressure gas stream produces a local pressure drop that draws the liquid composition into the stream of gases via capillary action. This fine liquid stream is then disintegrated by shear forces.

A nebulizer type inhalation delivery device can contain the compositions of the present disclosure as a solution, usually aqueous, or a suspension. For example, the composition can be suspended in saline and loaded into the inhalation delivery device. In generating the nebulized spray of the compositions for inhalation, the nebulizer delivery device may be driven ultrasonically, by compressed air, by other gases, electronically or mechanically (e.g., vibrating mesh or aperture plate). Vibrating mesh nebulizers generate fine particle, low velocity aerosol, and nebulize therapeutic solutions and suspensions at a faster rate than conventional jet or ultrasonic nebulizers. Accordingly, the duration of treatment can be shortened with a vibrating mesh nebulizer, as compared to a jet or ultrasonic nebulizer. Vibrating mesh nebulizers amenable for use with the methods described herein include the Philips Respironics I-Neb®, the Omron MicroAir, the Nektar Aeroneb®, and the PARI eFlow®. Other devices that can be used with the compositions described herein include jet nebulizers (e.g., PARI LC Star, AKITA), soft mist inhalers, and capsule-based dry powder inhalers (e.g., PH&T Turbospin).

The nebulizer may be portable and hand-held in design, and may be equipped with a self-contained electrical unit. The nebulizer device may comprise a nozzle that has two coincident outlet channels of defined aperture size through which the liquid composition can be accelerated. This results in impaction of the two streams and atomization of the composition. The nebulizer may use a mechanical actuator to force the liquid composition through a multiorifice nozzle of defined aperture size(s) to produce an aerosol of the composition for inhalation. In the design of single dose nebulizers, blister packs containing single doses of the composition may be employed.

The device can contain, and be used to deliver, a single dose of the compositions of the disclosure, or the device can contain, and be used to deliver, multi-doses of the compositions of the disclosure.

In the present disclosure the nebulizer may be employed to ensure the sizing of particles is optimal for positioning of the particle within, for example, the pulmonary membrane.

A metered dose inhalator (MDI) may be employed as the inhalation delivery device for the compositions of the present disclosure. This device is pressurized (pMDI) and its basic structure comprises a metering valve, an actuator and a container. A propellant is used to discharge the composition from the device. Suitable propellants, e.g., for MDI delivery, may be selected among such gases as fluorocarbons, chlorofluorocarbons (CFCs), hydrocarbons, hydrofluorocarbons, hydrofluoroalkane propellants (e.g., HFA-134a and HFA-227), nitrogen and dinitrogen oxide or mixtures thereof. Upon nebulization, the nebulized composition (also referred to as “aerosolized composition”) is in the form of aerosolized particles

In some embodiments, a compound of Formula II can be formulated into any suitable dosage form. Non-limiting examples of such dosage forms include aerosols, dispersions (e.g., aqueous oral dispersions, self-emulsifying dispersions, liposomal dispersions, dispersions with colloidal silica or nanospheres such as hydroxypropylmethylcellulose phthalate (HPMCP) nanospheres), pegylated liposomes, liquids, elixirs, suspensions (e.g., nanosuspensions), aerosols, controlled release formulations, lyophilized formulations, powders, delayed release formulations, extended release formulations, multiparticulate formulations, and mixed immediate release formulations. In some embodiments, a compound of Formula II, can be formulated for administration intranasally and/or by inhalation, e.g., using an inhalation device.

An “inhalation device,” as used herein, refers to any device that is capable of administering a drug formulation to the respiratory airways of a subject. Inhalation devices include conventional inhalation devices such as nebulizers, metered dose inhalers (MDIs), dry powder inhalers (DPIs), heat vaporizers, soft mist inhalers, thermal aerosol inhalers, or electrohydrodynamic-based solution misting inhalers. Inhalation devices also include nebulizers. “Nebulizer,” as used herein, refers to a device that turns medications, compositions, formulations, suspensions, and mixtures, etc. into a fine aerosol mist for delivery to the lungs. Non-limiting examples of nebulizers include jet nebulizers, mesh nebulizers, and ultrasonic wave nebulizers. Nebulizers, metered dose inhalers, and soft mist inhalers deliver pharmaceuticals by forming an aerosol which includes droplet sizes that can easily be inhaled. The aerosol can be used by a subject within the bounds of an inhalation therapy, whereby the compound of Formula II reaches the subject's respiratory tract upon inhalation. In some embodiments, the methods disclosed herein comprise administering to a subject a nominal dose of niclosamide or a pharmaceutically-acceptable salt thereof by an inhalation device, such as a nebulizer.

In some embodiments of the methods disclosed herein, administration of a composition comprising a compound of Formula II, to a subject with an inhalation device, e.g., a nebulizer, a metered dose inhaler, breath actuated device (BAI) such as a dry powder inhaler (DPI), a jet nebulizer, an ultrasonic wave nebulizer, a heat vaporizer, a soft mist inhaler, a thermal aerosol inhaler, or an electrohydrodynamic-based solution misting inhaler, is effective for the treatment or prophylaxis of COVID-19 in a subject.

Inhalation devices may be mechanical or electrical, and include, for example, jet nebulizers and ultrasonic nebulizers. Jet nebulizers generally utilize compressors to generate compressed air, which breaks the liquid medication into small breathable droplets, which form an aerosolized (atomized) mist. In some embodiments, when the subject breathes in, a valve at the top opens, which then allows air into the apparatus, thereby increasing the rate of mist generation; when the subject breathes out, the top valve closes, thereby slowing down mist generation while simultaneously permitting the subject to breathe out through the opening of a mouthpiece flap. Some nebulizers may provide the aerosol in a continuous mode (e.g., the eFlow from PARI Pharma Starnberg), by a breath enhanced mode (e.g., the PART LC Plus or Sprint from PARI Starnberg), by breath actuated mode dependent on the breathing pattern of the subject (e.g., the AeroEclipse from Trudell, Canada or the I-Neb from Philips Respironics), or according to given inhalation profile (e.g., the Akita from Activaero, Gmuenden, Germany).

Some conventional inhalation devices are disclosed in U.S. Pat. Nos. 9,566,399, 6,513,727, 6,513,519, 6,176,237, 6,085,741, 6,000,394, 5,957,389, 5,740,966, 5,549,102, 5,461,695, 5,458,136, 5,312,046, 5,309,900, 5,280,784, and 4,496,086, and International Publication Nos. WO 2018/191776 and WO 2018/213834, each of which is hereby incorporated by reference in its entirety. Commercial conventional inhalation devices are available from: PARI (Germany) under the trade names PARI LC Plus®, LC Star®, and PARI-Jet®; A & H Products, Inc. (Tulsa, Okla.) under the trade name AquaTower®; Hudson RCI (Temecula, Calif.) under the trade name AVA-NEB®; Intersurgical, Inc. (Liverpool, N.Y.) under the trade name Cirrus®; Salter Labs (Arvin, Calif.) under the trade name Salter 8900@; Respironics (Murrysville, Pa.) under the trade name Sidestream®; Bunnell (Salt Lake City, Utah) under the trade name Whisper Jet®; Smiths-Medical (Hyth Kent, UK) under the trade name Downdraft®, and DeVilbiss (Somerset, Pa.) under the trade name DeVilbiss®; or Trudell, Canada under the trade name AeroEclipse®.

In some embodiments of the methods disclosed herein, compositions comprising a compound of Formula II are administered with a thermal aerosol inhaler.

Nebulizers are inhalation devices that comprise a micro-perforated membrane through which a liquid solution is converted through electrical or mechanical means into aerosol droplets suitable for inhalation. Nebulizers can deliver a large fraction of a loaded dose to a subject. In some embodiments, the nebulizer also utilizes one or more actively or passively vibrating microperforated membranes. In some embodiments, the nebulizer contains one or more oscillating membranes. In some embodiments, the nebulizer contains a vibrating mesh or plate with multiple apertures and optionally a vibration generator with an aerosol mixing chamber. In some such embodiments, the mixing chamber functions to collect (or stage) the aerosol from the aerosol generator. In some embodiments, an inhalation valve is also used to allow an inflow of ambient air into the mixing chamber during an inhalation phase and is closed to prevent escape of the aerosol from the mixing chamber during an exhalation phase. In some such embodiments, the exhalation valve is arranged at a mouthpiece which is removably mounted at the mixing chamber and through which the subject inhales the aerosol from the mixing chamber. Still yet, in some embodiments, the nebulizer contains a pulsating membrane. In some embodiments, the nebulizer is continuously operating.

In some embodiments, the nebulizer contains a vibrating micro-perforated membrane of tapered nozzles that generates a plume of droplets without the need for compressed gas. In these embodiments, a solution in the micro-perforated membrane nebulizer is in contact with a membrane, the opposite side of which is open to the air. The membrane is perforated by a large number of nozzle orifices of an atomizing head. An aerosol is created when alternating acoustic pressure in the solution is built up in the vicinity of the membrane causing the fluid on the liquid side of the membrane to be emitted through the nozzles as uniformly sized droplets.

Some embodiments of nebulizers use passive nozzle membranes and a separate piezoelectric transducer that stimulates the membrane. In contrast, some nebulizers employ an active nozzle membrane, which use the acoustic pressure in the nebulizer to generate very fine droplets of solution via the high frequency vibration of the nozzle membrane.

Some nebulizers can contain a resonant system. For example, in such nebulizers, the membrane is driven by a frequency for which the amplitude of the vibrational movement at the center of the membrane is particularly large, resulting in a focused acoustic pressure in the vicinity of the nozzle; the resonant frequency may be about 100 kHz. A flexible mounting is used to keep unwanted loss of vibrational energy to the mechanical surroundings of the atomizing head to a minimum. In some embodiments, the vibrating membrane of the nebulizer may be made stainless steel, or of a nickel-palladium alloy by electroforming.

Commercial high efficiency nebulizers are available from: PARI (Germany) under the trade name eFlow®; Aerogen, Ltd. (Ireland) under the trade names AeroNeb® Go and AeroNeb® Pro, AeroNeb® Solo, and other nebulizers utilizing the OnQ® nebulizer technology; Respironics (Murrysville, Calif) under the trade names I-Neb®; Omron (Bannockburn, Ill.) under the trade name Micro-Air®; Activaero (Germany) under the trade name Akita®, and AerovectRx (Atlanta, Ga.) under the trade name AerovectRx®.

In some embodiments, a composition comprising a compound of Formula II, is formulated as an inhalable nanosuspension (see, e.g., Costabile et al. Mol Pharm. 2015 Aug. 3; 12(8):2604-17.) In some embodiments, an inhalable nanosuspension of a compound of Formula II, is administered to a subject using a nebulizer.

In some embodiments, devices for intranasal administration of a compound of Formula II, include one or more features present in any inhalation device described herein. In some embodiments, devices for intranasal administration of a compound of Formula II, are spray devices. Suitable commercially available nasal spray devices include Accuspray™ (Becton Dickinson). In some embodiments, spray devices for intranasal use are devices for which the performance of the device is not dependent upon the pressure applied by the user. These devices are known as pressure threshold devices. Pressure threshold devices release liquid from the nozzle only when a threshold pressure is applied. These devices make it easier to achieve a spray with a regular droplet size. Pressure threshold devices suitable for use with the present invention are known in the art and are described for example in WO 91/13281, EP 311863, and EP 516636. Pressure threshold devices are commercially available from Pfeifer GmbH and are also described in Bommer, R. Pharmaceutical Technology Europe, September 1999.

In some embodiments, the intranasal devices can administer a compound of Formula II, by means of bi-dose delivery. Bi-dose devices contain two sub-doses of a single dose, one sub-dose for administration to each nostril. Generally, the two sub-doses are present in a single chamber and the construction of the device allows for efficient delivery of a single sub-dose at a time. Alternatively, a monodose device may be used for administering the composition according to the invention.

In some embodiments, a compound of Formula II, is formulated as an ointment or gel for intranasal delivery.

In some embodiments, the compositions disclosed herein can include one or pharmaceutical excipients that provide suitable properties for intranasal administration and/or administration by inhalation of a compound of Formula II. See, e.g., Labiris and Dolovich, Br J Clin Pharmacol. 2003 December; 56(6): 600-612. Non-limiting examples of such pharmaceutical excipients can include surfactants, tonicity agents, suspending agents, viscosity enhancing agents, wetting agents, co-solvents, solubilizers, pH modulators, antimicrobial agents, antioxidants/stabilizers, bulking agents/diluents and propellants.

“Suspending agents” include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

“Surfactants” include compounds such as sodium lauryl sulfate, sodium docusate, Tween 20, 60 or 80, triacetin, vitamin E TPGS, sorbitan monooleate, polyoxyethylene sorbitan monooleate, polysorbates, poloxamers, (Pluronic F68 and F127), bile salts, glyceryl monostearate, copolymers of ethylene oxide and propylene oxide, e.g., Pluronic® (BASF), Triton X-100, Brij 30, Brij 35 and the like. Additional examples of surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

“Viscosity enhancing agents” include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans, polyethylene glycol (PEG 400-3350), and combinations thereof.

“Wetting agents” include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 20, Tween 80, Triton X-100, Brij 30, Brij 35, vitamin E TPGS, ammonium salts and the like.

“Tonicity agents” include sodium chloride, potassium chloride, Mannitol, Sorbitol, Lactose, Dextrose, Trehalose, Glycerol, Glycerin and like.

“pH modifiers” include hydrochloric acid, sodium hydroxide, Acetate, Citrate, citric acid, Tartrate, Histidine, Glutamate, Phosphate, Tris, Glycine, Bicarbonate, Succinate, Sulfate, Nitrate and the like.

“Co-solvents” include PEG (400-3350), alcohol, propylene glycol and the like.

“Solubilizers” include PEG (400-3350), propylene glycol, oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, triacetin, Tween 20, Tween 80, Triton X-100, Brij 30, Brij 35, vitamin E TPGS, ammonium salts and the like.

“Antimicrobial agents” include benzyl alcohol, benzalkonium chloride, metacresol, phenol, 2-phenoxyethanol and the like.

“Bulking agents and diluents” include sucrose, Trehalose, Glucose, Lactose, Sorbitol, Mannitol, Glycerol, Gelatin, PVP, PLGA, PEG, dextran, cyclodextrin and derivatives, starch derivatives, HSA, BSA, water, liquid carbon dioxide, alcohol and the like.

“Antioxidants and stabilizers” include histamine, methionine, ascorbic acid, glutathione, vitamin E, poly(ethylenimine) and the like.

Non-limiting examples of propellants include chlorofluorocarbons (CFC) and hydrofluoroalkanes (HFAs)

Compositions and formulations of the disclosure may have a surface tension effective for deposition, penetration or retention of the composition primarily in the peripheral lung regions, including the bronchioles and alveoli.

### Compounds

In some aspects, the present disclosure is directed to methods of treating and/or preventing a SARS-CoV-2 infection and/or a SARS-CoV-2 infection-associated disease or disorder (e.g. COVID-19) with one or more compounds of Formula I:

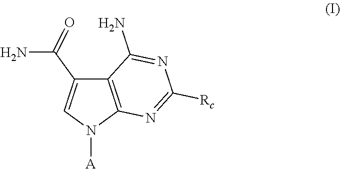

prodrugs thereof, metabolites thereof, and pharmaceutically acceptable enantiomers, diastereomers, racemates, mixtures, solvates or salts thereof, wherein R_{c} and A are as defined above.

In some aspects, the present disclosure is directed to treating and/or preventing a SARS-CoV-2 infection and/or a SARS-CoV-2 infection-associated disease or disorder (e.g. COVID-19) with one or more compounds of Formula I, wherein A is selected from A1 through A14:

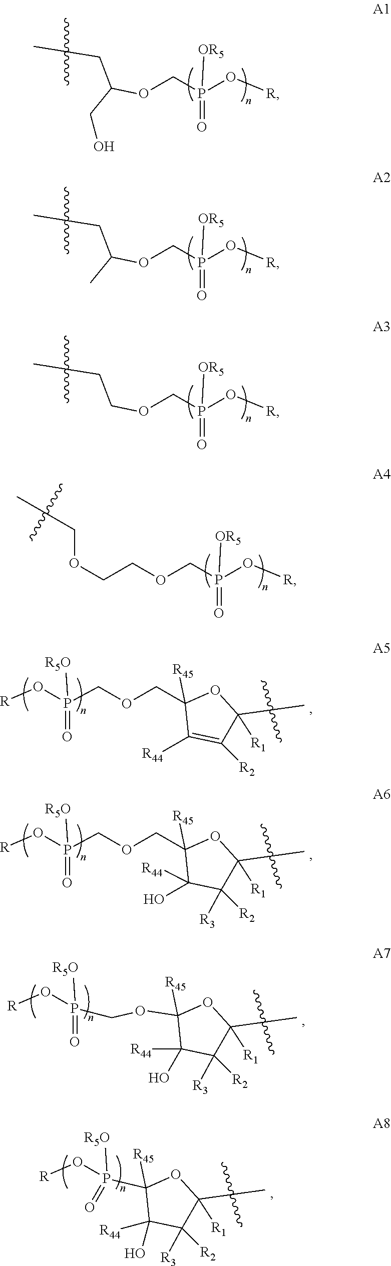

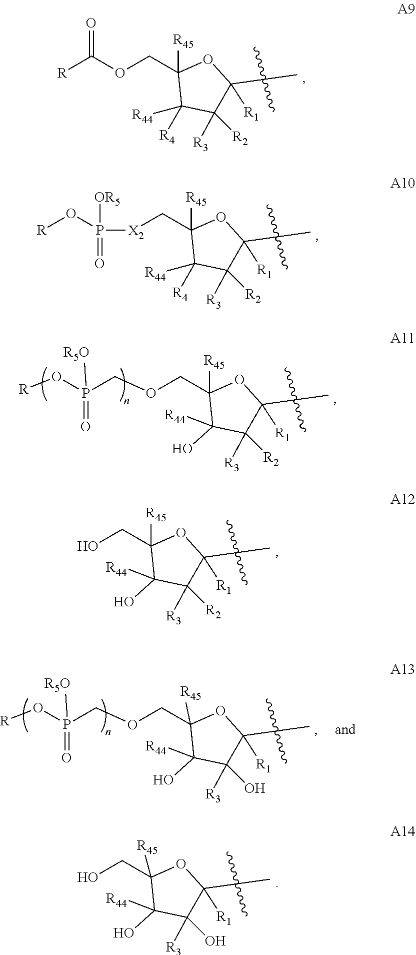

In one or more embodiments, A is:

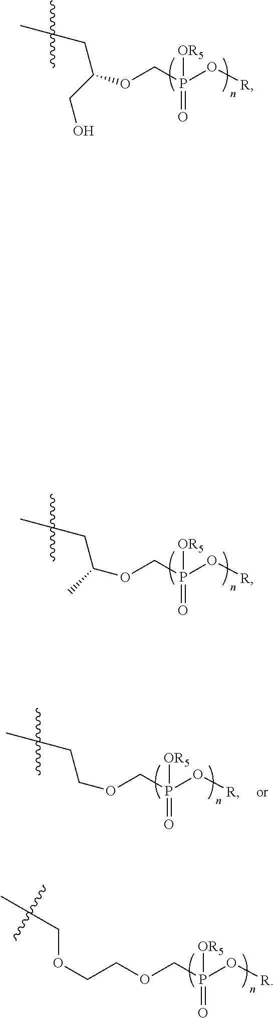

In one or more embodiments, A is:

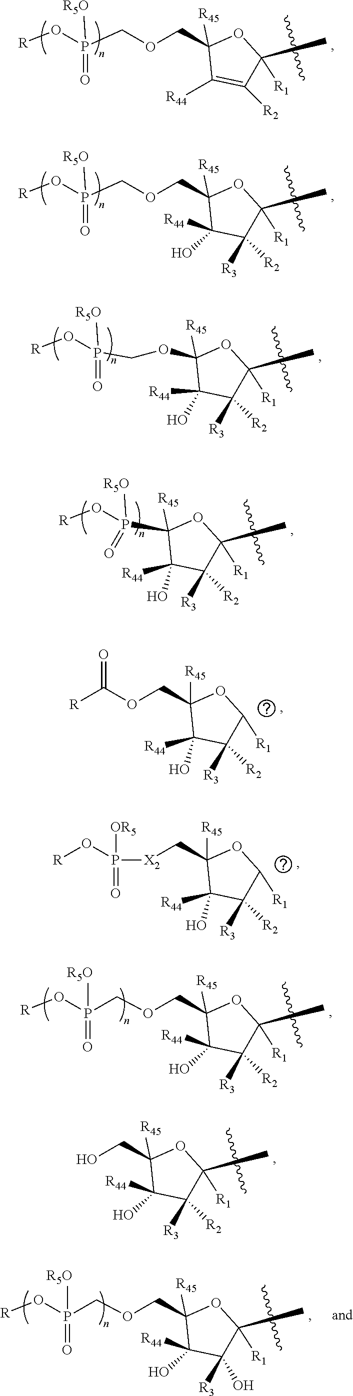

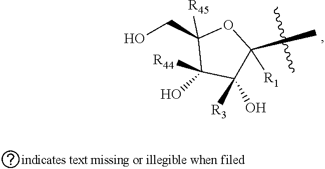

In one or more embodiments, A is

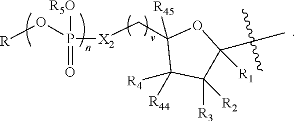

In one or more embodiments, A is

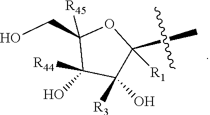

In one or more embodiments, compounds of Formula I can have one or more of the following features. R₄ is H, substituted or unsubstituted C₁-C₆ alkyl, NH₂, OH, or SH. R₄ can be C₁-C₆ alkyl optionally substituted with one or more halogens. R₄ can be methyl, CH₂X, CHX₂ or CX₃, wherein X is halogen. R₄ can be pentyl. R₁ can be hydrogen. R₂ and R₃ can each independently be hydrogen, CH₃, CH₂X, CHX₂, CX₃, N₃, OH, or NH₂, wherein X is halogen. R₅ can be H, M, C₁-C₆ alkyl, phenyl, or benzyl. M⁺ can be Na⁺, Li⁺, K⁺, Ca²⁺, Mg²⁺, or NR_{g}R_{d}RₑR_{f}⁺, wherein R_{g}, R_{d}, Rₑ and R_{f} are each independently hydrogen or C₁₋₅ alkyl. R can be H. When X₂ is absent, v can be 0. R₁ can be hydrogen. R₄₅ can be hydrogen. R_{c} can be C₁-C₆ alkyl, e.g., methyl.

In one or more embodiments, of the compounds of Formula I, or Formula IA, R₁ is —H. In one or more embodiments, R₂ is —OH. In one or more embodiments, R₄ is —OH. In one or more embodiments, R₂ and R₄ are each —OH. In one or more embodiments, R₃ is —H. In one or more embodiments, R₄₄ is —H. In one or more embodiments, R₃ and R₄₄ are each —H. In one or more embodiments, R is —H. In one or more embodiments R_{c} is —CH₃. In one or more embodiments, v is 1, X₂ is —O—, n is 0, and R is —H.

In one or more embodiments of the compounds of Formula II, R^{a} and R^{b} are both —H. In one or more embodiments, R^{c} is —H. In one or more embodiments, n is 0. In one or more embodiments, R is —H. In one or more embodiments, R^{a}, R^{b}, and R^{c} are —H. In one or more embodiments, R^{a}, R^{b}, and R^{c} are —H and n is 0. In one or more embodiments, R^{a}, R^{b}, and R^{c} are —H. In one or more embodiments, R^{a}, R^{b}, and R^{c} are —H, n is 0 and R is —H.

In one or more embodiments, the present disclosure relates to methods of treating and/or preventing a SARS-CoV-2 infection and/or a SARS-CoV-2 infection-associated disease or disorder (e.g. COVID-19) with one or more compounds of Formula IB:

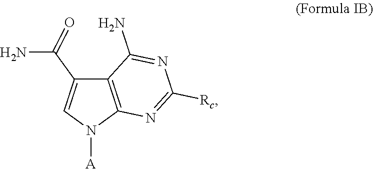

prodrugs thereof, metabolites thereof, and pharmaceutically acceptable salts, solvates, enantiomers, diastereomers, racemates or mixtures thereof, wherein:
A is:

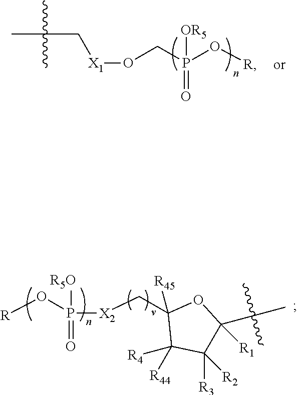

X₁ is —CR₁₁R₁₂— or —OCH₂CH₂— wherein the oxygen atom is distal to the R moiety in A;
R₁₁ and R₁₂ are independently hydrogen or C₁-C₄ alkyl, wherein the alkyl is optionally substituted with one or more halogen, —OH, —SH, or —NH₂;
X₂ is absent, —O—, —C(O)O—, or —OCH₂— wherein the oxygen atom is distal to the R moiety in A;
each R independently is hydrogen, —C₁-C₆alkyl,

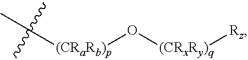

or an amino acid residue bound via the carbonyl group, wherein the alkyl is optionally substituted with one or more halogen, —OH, —SH, or —NH₂;
v is 0 or 1;
n is 0, 1, 2, or 3 and when X₂ is —C(O)O—, n is 0;
p is 2, 3, 4, 5, 6, 7, 8, 9, 10, or 11;
q is 1, 2, 3, 4, 5, 6, 7, 8, 9, 10, 11, 12, 13, 14, 15, 16, 17, or 18;
R_{z} is hydrogen, halogen, —C₁-C₄ alkylthio, —C₁-C₄ alkoxy, —C₁-C₄alkyl, —C₂-C₄alkenyl, —C₂-C₄alkynyl, aryl, heteroaryl, —C₃-C₈cycloalkyl, —C₄-C₈cycloalkenyl, or 3- to 5-membered nonaromatic heterocycle, wherein each alkylthio, alkoxy, alkyl, alkenyl, alkynyl, aryl, heteroaryl, cycloalkyl, cycloalkenyl, or heterocycle is optionally substituted with one or more halogen, —OH, —SH, or —NH₂;
Rₐ, R_{b}, Rₓ, and R_{y} are each independently selected from the group consisting of hydrogen, halogen, —OH, —SH, —C₁-C₆ alkoxy, aryloxy, —C₁-C₆alkylthio, arylthio, —OC(O)C₁-C₆alkyl, —OC(O)aryl, —C₁-C₆alkyl, —C₂-C₆ alkenyl, —C₂-C₆alkynyl, aryl, heteroaryl, —C₃-C₈cycloalkyl, and —C₄-C₈cycloaklenyl, wherein each alkoxy, aryloxy, alkylthio, arylthio, alkyl, aryl, alkenyl, alkynyl, heteroaryl, cycloalkyl, or cycloalkenyl is optionally substituted with one or more halogen, —OH, —SH, or —NH₂;
or any two Rₐ or R_{b}, together with the atom to which they are both attached, can combine to form a C₃-C₈ spirocycloalkyl or 3- to 8-membered spiroheterocycle;
or any two Rₐ or R_{b}, when on adjacent atoms, can combine to form a cis- or trans-carbon-carbon double bond or a carbon-carbon triple bond;
or any two Rₐ or R_{b}, when on adjacent atoms, can combine to form an aryl, heteroaryl, —C₃-C₁₀cycloalkyl, —C₄-C₁₀cycloalkenyl, or 5- to 10-membered ring heterocycle;
or any CRₐR^{b} can be replaced by —O—, —S—, —S(O)—, or —SO₂—;
or any two Rₓ or R_{y}, together with the atom to which they are both attached, can combine to form a —C₃-C₈ spirocycloalkyl or 3- to 8-membered spiroheterocycle;
or any two Rₓ or R_{y}, when on adjacent atoms, can combine to form a cis- or trans-carbon-carbon double bond or a carbon-carbon triple bond;
or any two Rₓ or R_{y}, when on adjacent atoms, can combine to form an aryl, heteroaryl, —C₃-C₁₀cycloalkyl, —C₄-C₁₀cycloalkenyl, or 5- to 10-membered ring heterocycle;
or any CR_{X}R_{y} can be replaced by —O—, —S—, —S(O)—, or —SO₂—;
R₁ and R₄₅ are each independently hydrogen, halogen, —N₃, —OH, —NH₂, —SH, —C₁-C₆alkyl, —C₃-C₆ cycloalkyl, —C₂-C₆ alkenyl, —C₄-C₈ cycloalkenyl, —C₂-C₆ alkynyl, —C₈-C₁₂ cycloalkynyl, —C₁-C₆alkoxy, or —C₁-C₆ alkylthio wherein each alkyl, cycloalkyl, alkenyl, cycloalkenyl, alkynyl, cycloalkynyl, alkoxy or alkylthio is independently substituted with one or more halogen, —N₃, —OH, —NH₂, or —SH;
R₂, R₃, R₄ and R₄₄ are each independently hydrogen, halogen, —N₃, —OH, —NH₂, —SH, —C₁-C₆alkyl, —C₁-C₆alkoxy, or —C₁-C₆alkylthio, wherein each alkyl, alkoxy, or alkylthio is optionally substituted with one or more halogen, —N₃, —OH, —NH₂, or —SH;
or R₃ and one of R₄ and R₄₄, together with the atoms to which they are attached can form a carbon-carbon double bond;
R₅ is independently hydrogen, —R, M⁺, aryl, aralkyl, —C₁-C₆ alkyl, —C₁-C₆ heteroalkyl, cycloalkyl, non-aromatic heterocyclic ring, or heteroaryl, wherein M⁺ is a cation and wherein each aryl, aralkyl, alkyl, heteroalkyl, cycloalkyl, heterocycle, or heteroaryl is optionally substituted with one or more halogen, —N₃, —OH, —NH₂, or —SH; and
R_{c} is —C₁-C₆ alkyl, —C₃-C₆ cycloalkyl, —C₂-C₆ alkenyl, —C₄-C₈ cycloalkenyl, —C₂-C₆ alkynyl, —C₈-C₁₂ cycloalkynyl, or aryl, wherein each alkyl, cycloalkyl, alkenyl, cycloalkenyl, or aryl is optionally substituted with one or more halogen, —N₃, —OH, —NH₂, or —SH.

In one or more embodiments, R is selected from:

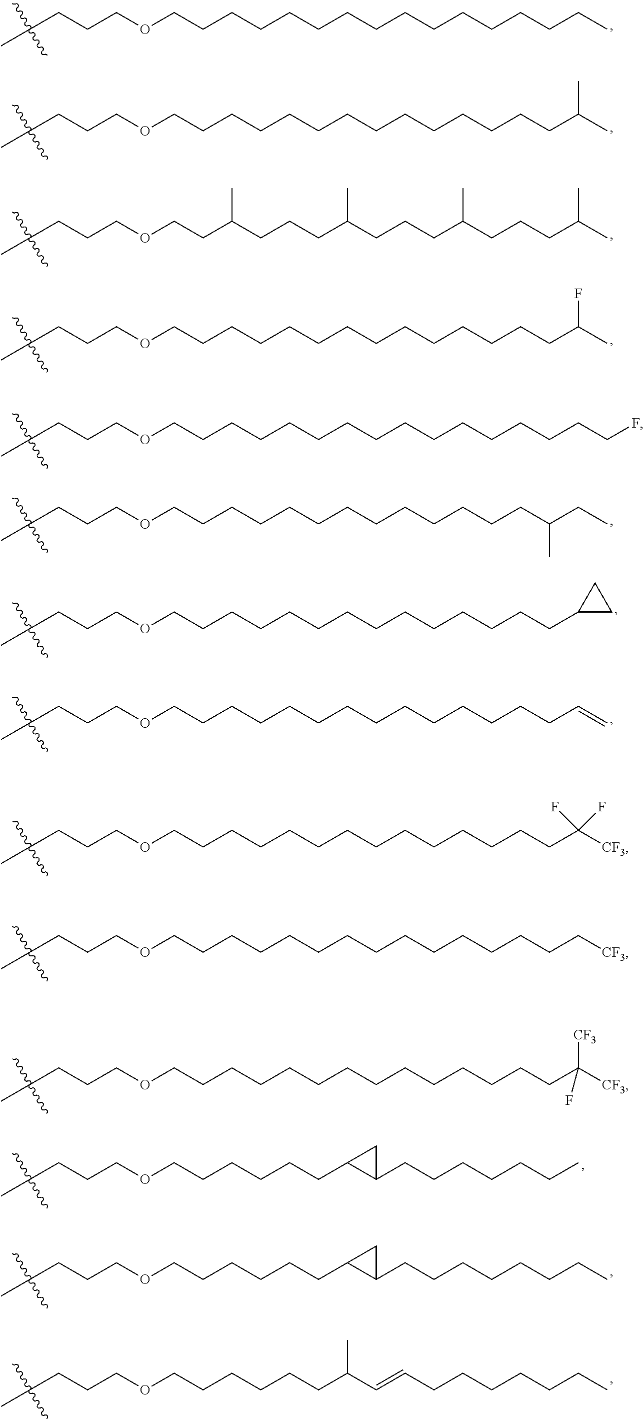

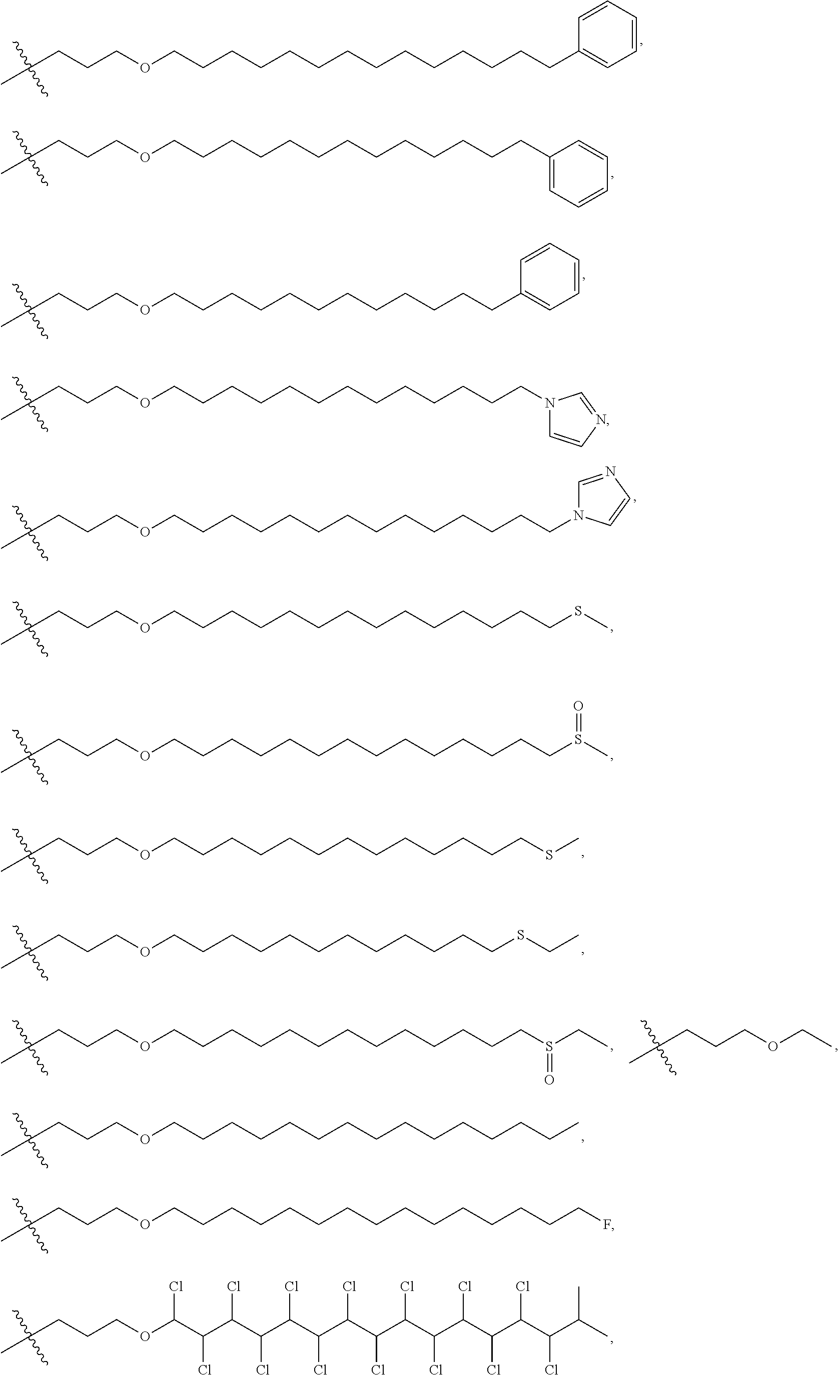

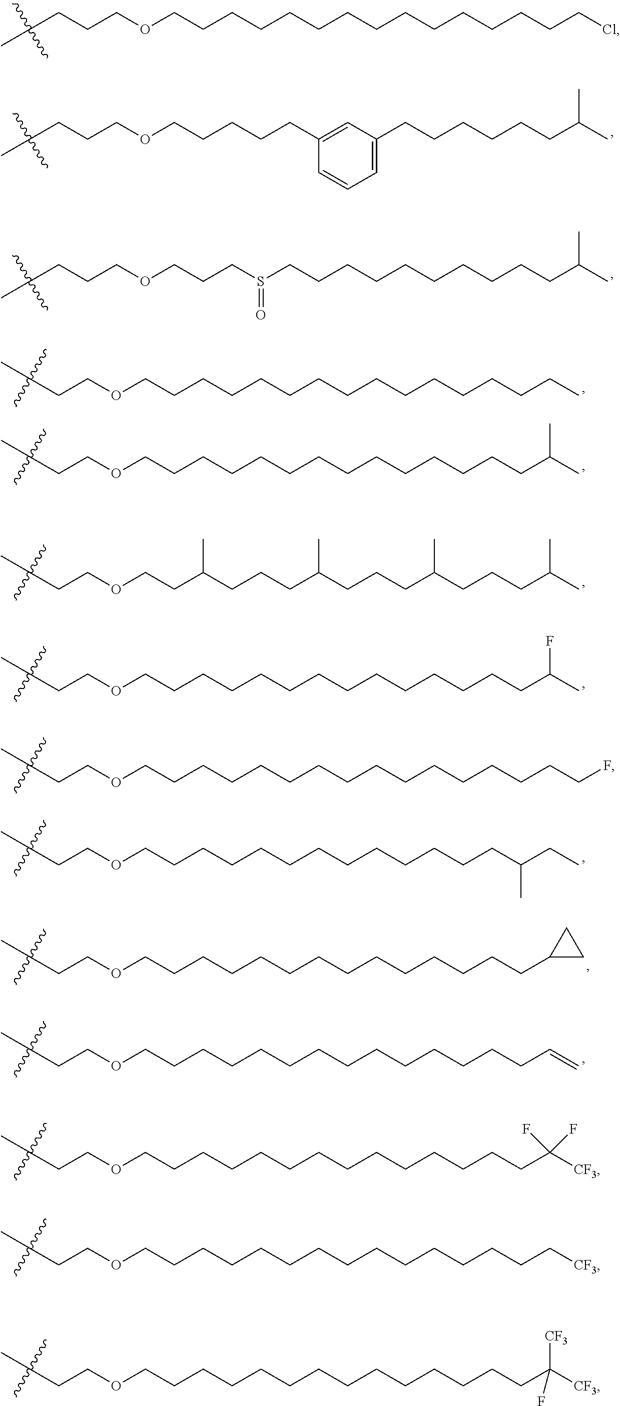

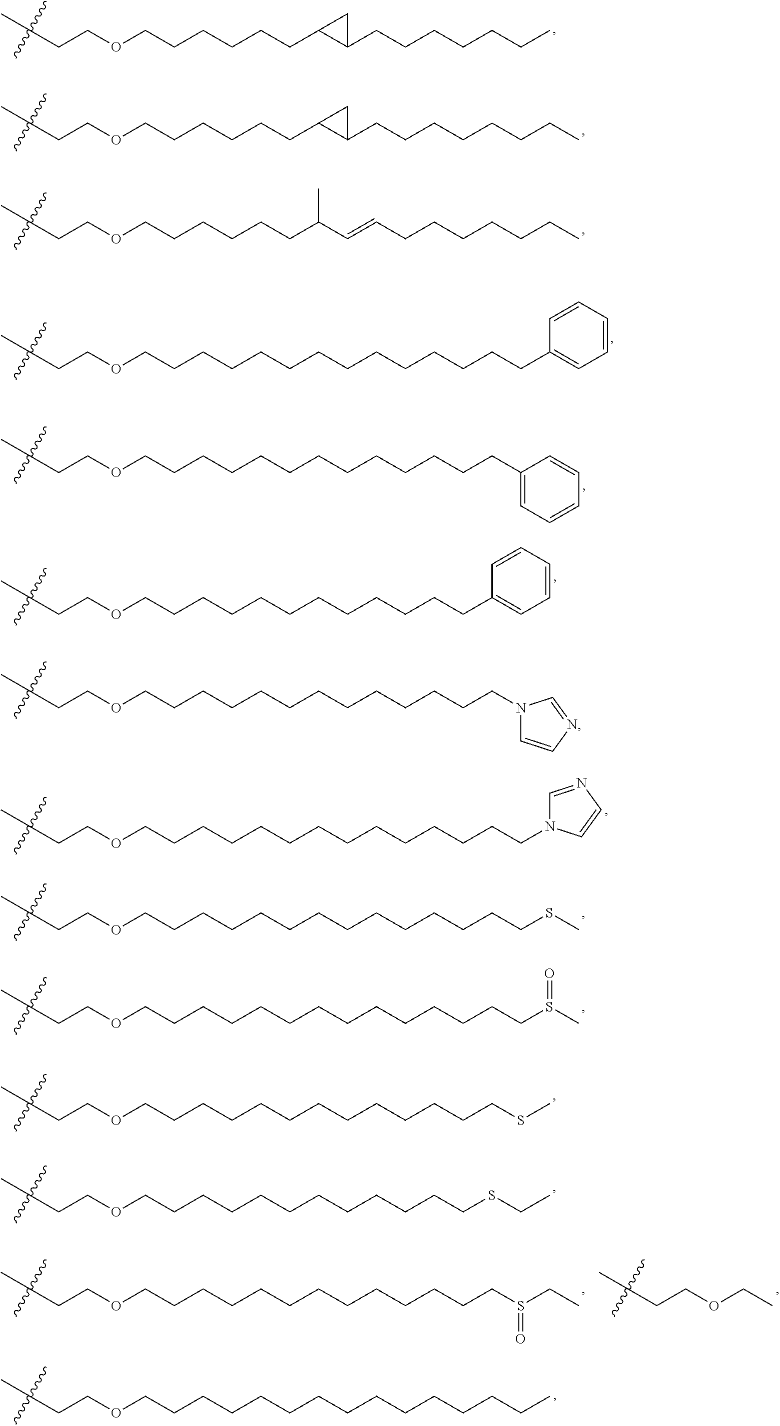

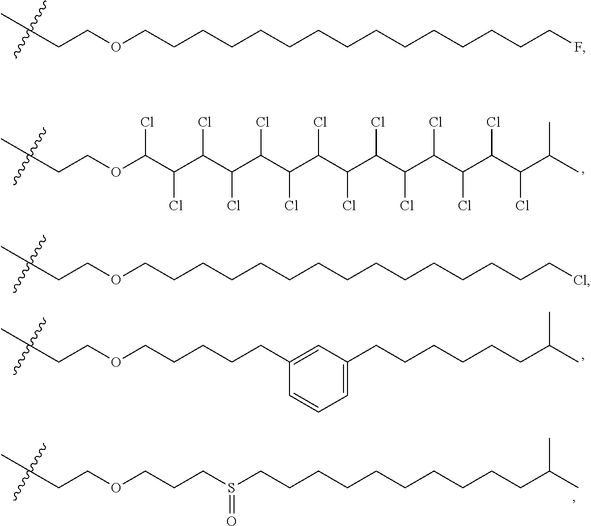

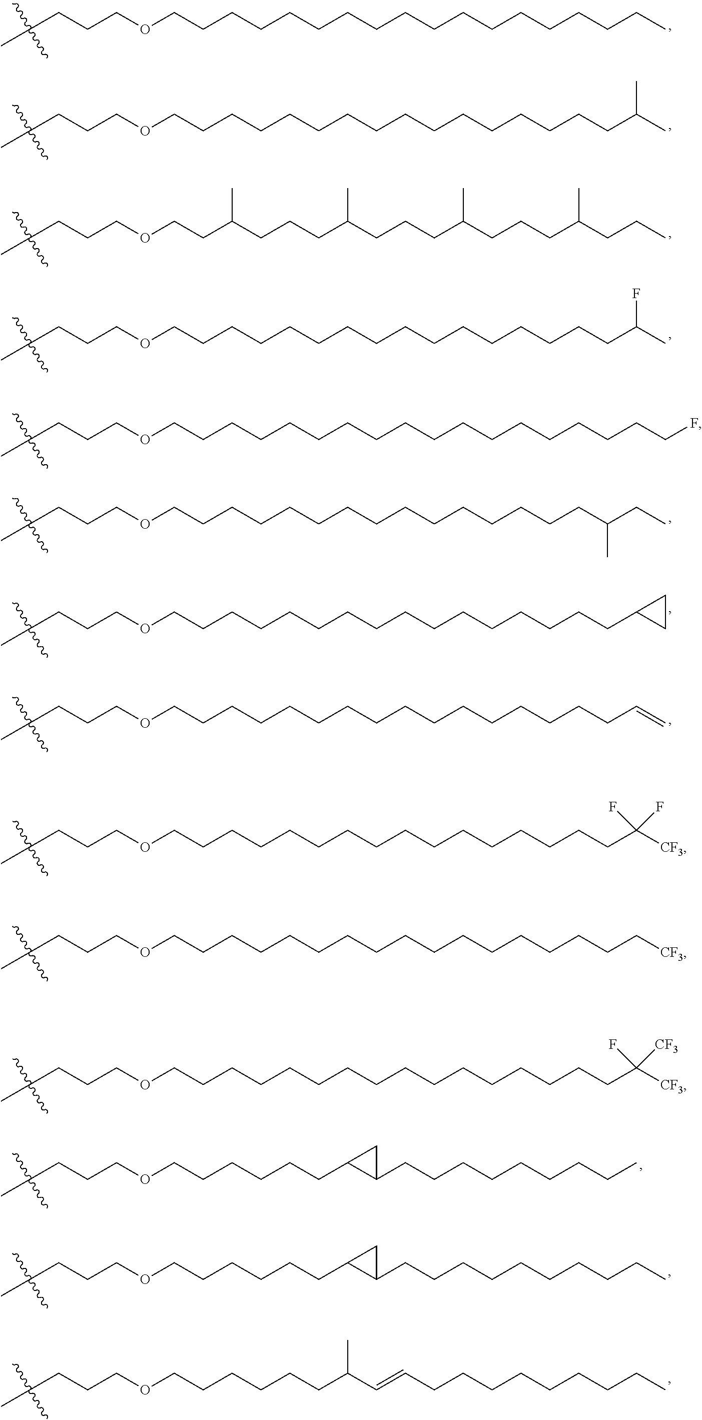

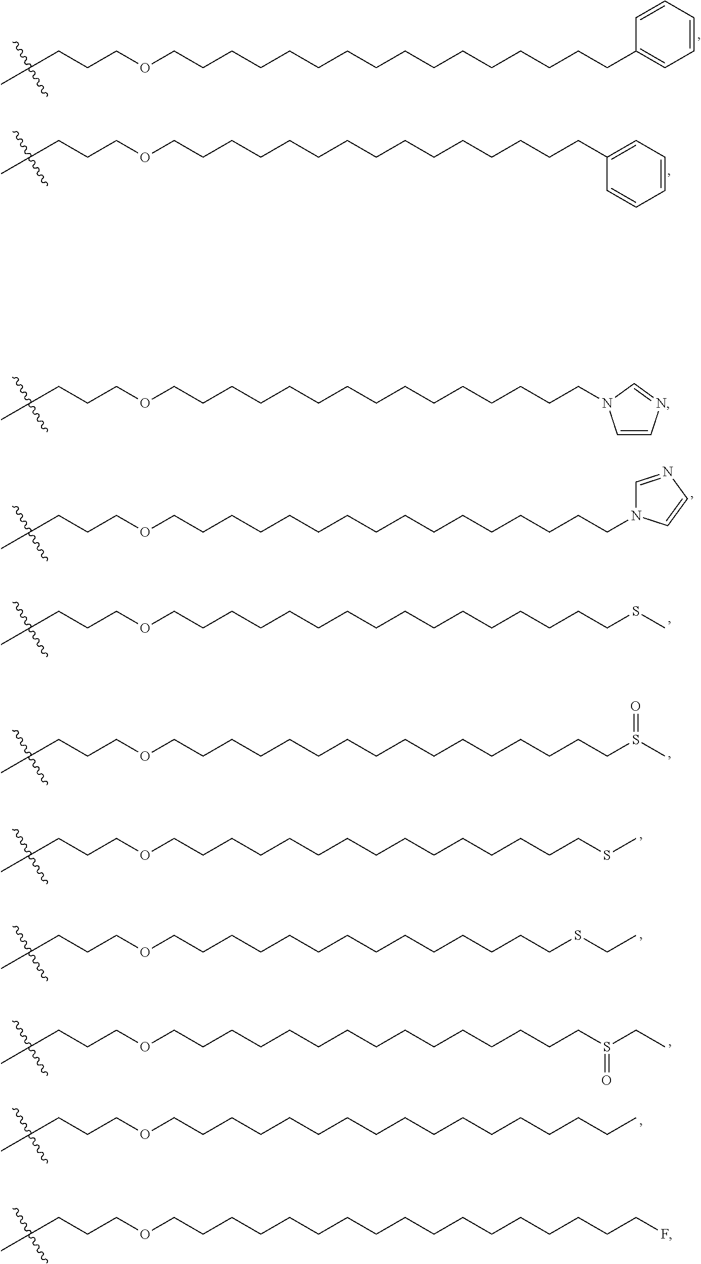

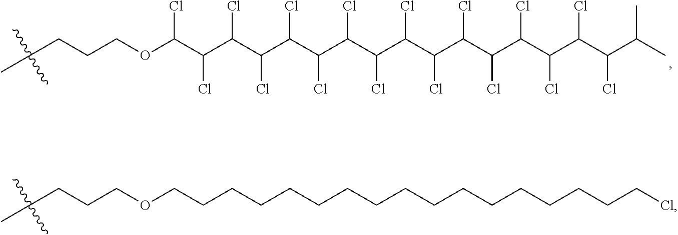 [SMILES: *CCCOC(Cl)C(Cl)C(Cl)C(Cl)C(Cl)C(Cl)C(Cl)C(Cl)C(Cl)C(Cl)C(Cl)C(Cl)C(Cl)C(Cl)C(Cl)C(Cl)C(C)C ; *CCCOCCCCCCCCCCCCCCCCCCl]

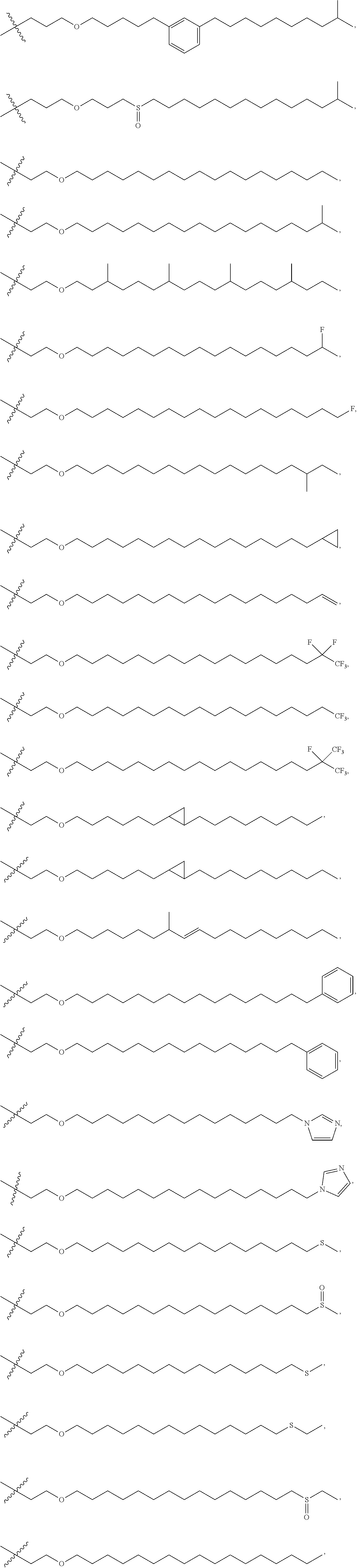

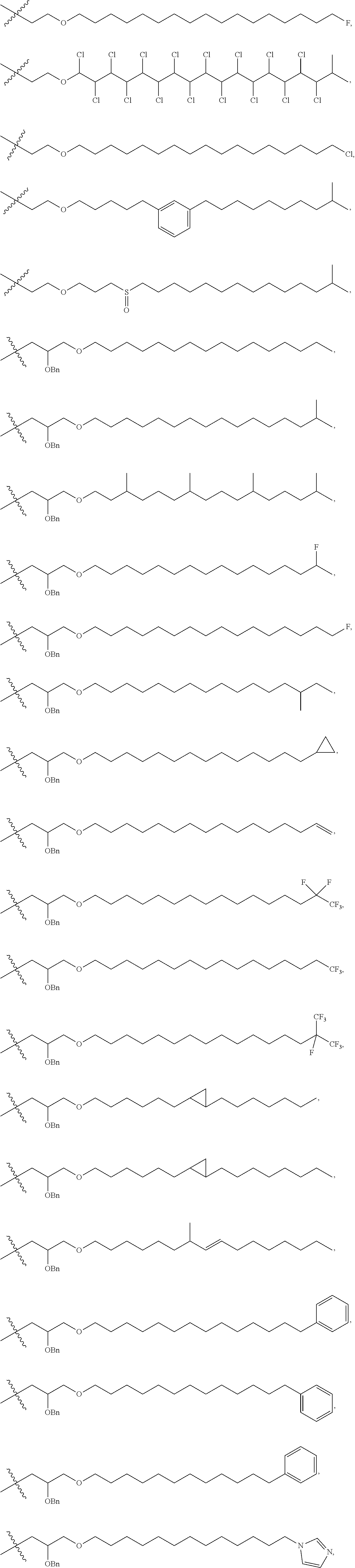

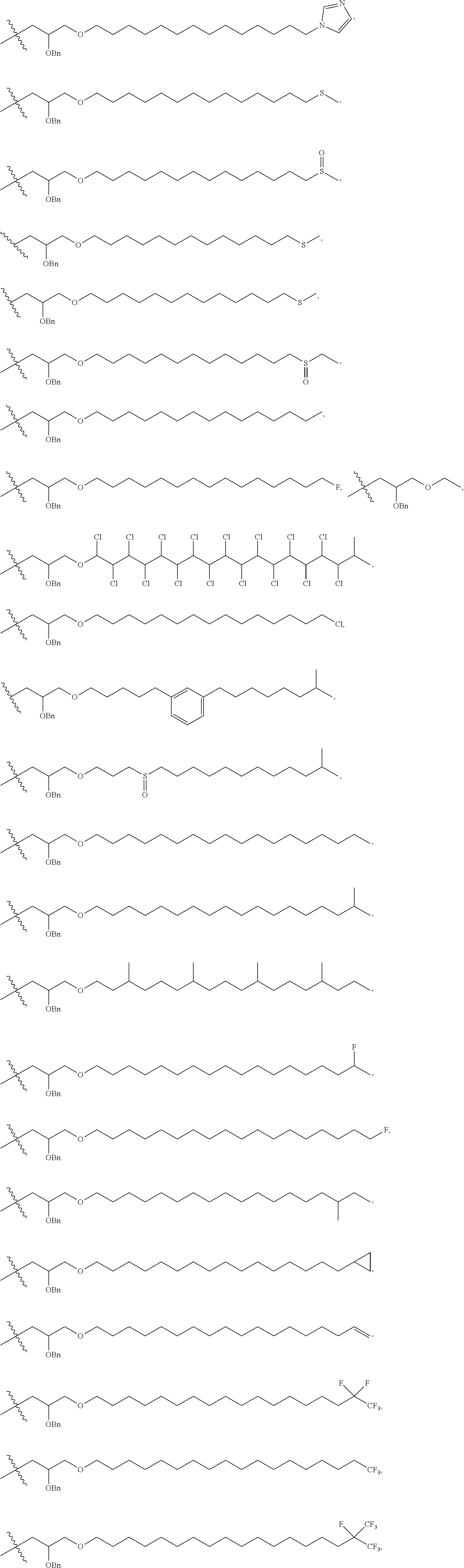

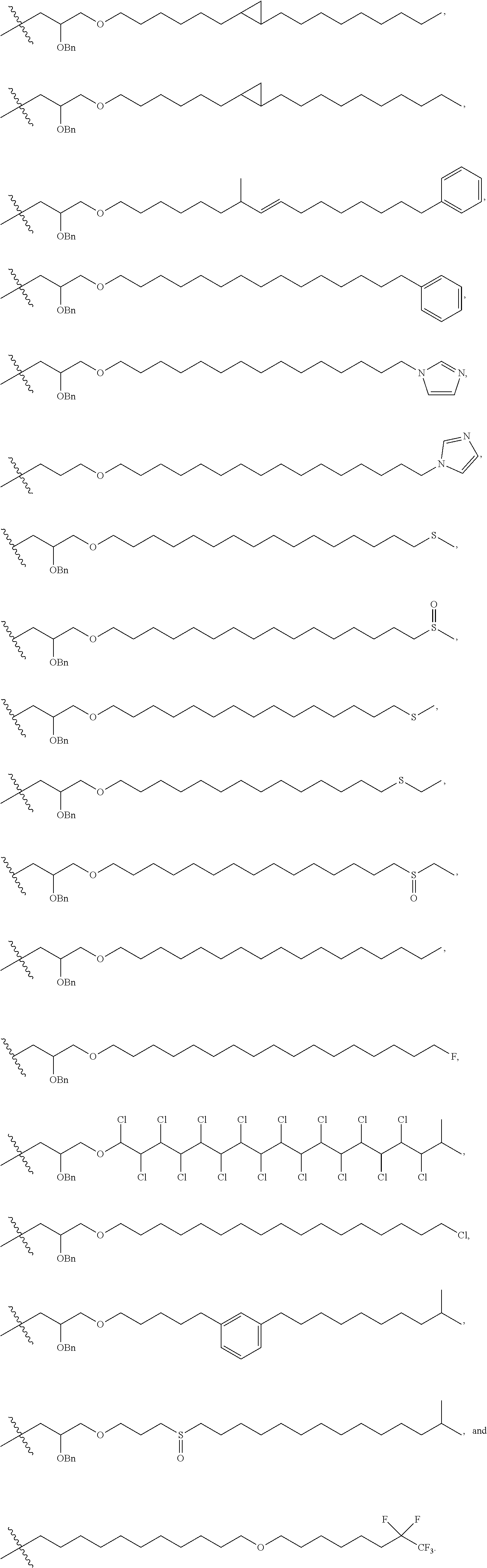

In one or more embodiments, the present disclosure relates to methods of treating and/or preventing a SARS-CoV-2 infection and/or a SARS-CoV-2 infection-associated disease or disorder (e.g. COVID-19) with one or more compounds of Formula I-A:

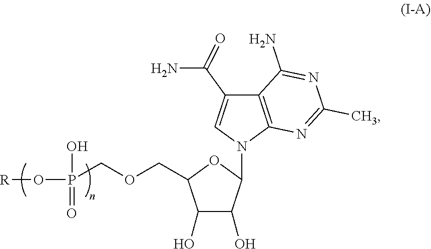

prodrugs thereof, or metabolites thereof.

In one or more embodiments, the present disclosure relates to methods of treating and/or preventing a SARS-CoV-2 infection and/or a SARS-CoV-2 infection-associated disease or disorder (e.g. COVID-19) with one or more compounds of Formula I-B:

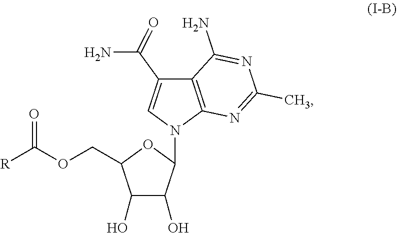

prodrugs thereof, or metabolites thereof.

In one or more embodiments, the present disclosure relates to methods of treating and/or preventing a SARS-CoV-2 infection and/or a SARS-CoV-2 infection-associated disease or disorder (e.g. COVID-19) with one or more compounds of Formula I-C:

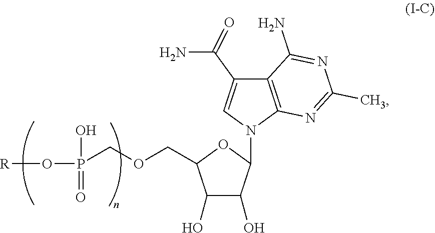

prodrugs thereof, or metabolites thereof.

In one or more embodiments, the present disclosure relates to methods of treating and/or preventing a SARS-CoV-2 infection and/or a SARS-CoV-2 infection-associated disease or disorder (e.g. COVID-19) with one or more compounds of Formula I-D:

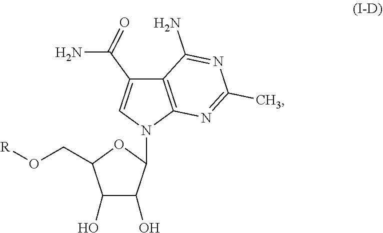

prodrugs thereof, or metabolites thereof.

In one or more embodiments, the present disclosure relates to methods of treating and/or preventing a SARS-CoV-2 infection and/or a SARS-CoV-2 infection-associated disease or disorder (e.g. COVID-19) with one or more compounds of is:

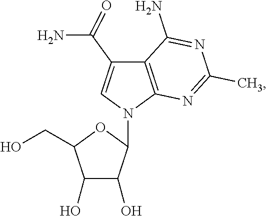 [SMILES: Cc1nc(N)c2c(C(N)=O)cn(C3OC(CO)C(O)C3O)c2n1]

a prodrug thereof, or a metabolite thereof.

In one or more embodiments, the present disclosure relates to methods of treating and/or preventing a SARS-CoV-2 infection and/or a SARS-CoV-2 infection-associated disease or disorder (e.g. COVID-19) with Compound 1:

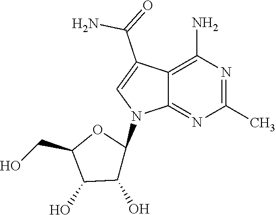 [SMILES: Cc1nc(N)c2c(C(N)=O)cn([C@@H]3O[C@H](CO)[C@@H](O)[C@H]3O)c2n1]

(Compound 1; 4-amino-7-((2R,3R,4S,5R)-3,4-dihydroxy-5-(hydroxymethyl)tetrahydrofuran-2-yl)-2-methyl-7H-pyrrolo[2,3-d]pyrimidine-5-carboxamide), a prodrug thereof, or a metabolite thereof.

In one or more embodiments, the compound of the disclosure is Compound 1-triphosphate (Compound 1-TP or Compound 1-PPP):

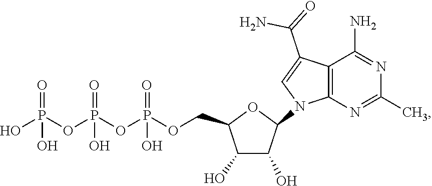 [SMILES: Cc1nc(N)c2c(C(N)=O)cn([C@@H]3O[C@H](COP(=O)(O)OP(=O)(O)OP(=O)(O)O)[C@@H](O)[C@H]3O)c2n1]

a prodrug thereof, or a metabolite thereof.

In one or more embodiments, the present disclosure relates to methods of treating and/or preventing a SARS-CoV-2 infection and/or a SARS-CoV-2 infection-associated disease or disorder (e.g. COVID-19) with one or more of:

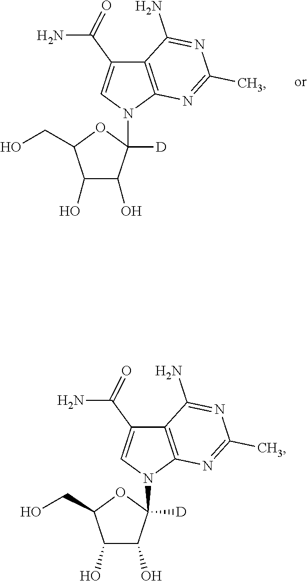 [SMILES: [2H]C1(n2cc(C(N)=O)c3c(N)nc(C)nc32)OC(CO)C(O)C1O ; "or" ; [2H][C@@]1(n2cc(C(N)=O)c3c(N)nc(C)nc32)O[C@H](CO)[C@@H](O)[C@H]1O]

a prodrug thereof, or a metabolite thereof

In one or more embodiments, the present disclosure relates to methods of treating and/or preventing a SARS-CoV-2 infection and/or a SARS-CoV-2 infection-associated disease or disorder (e.g. COVID-19) with one or more compounds of:

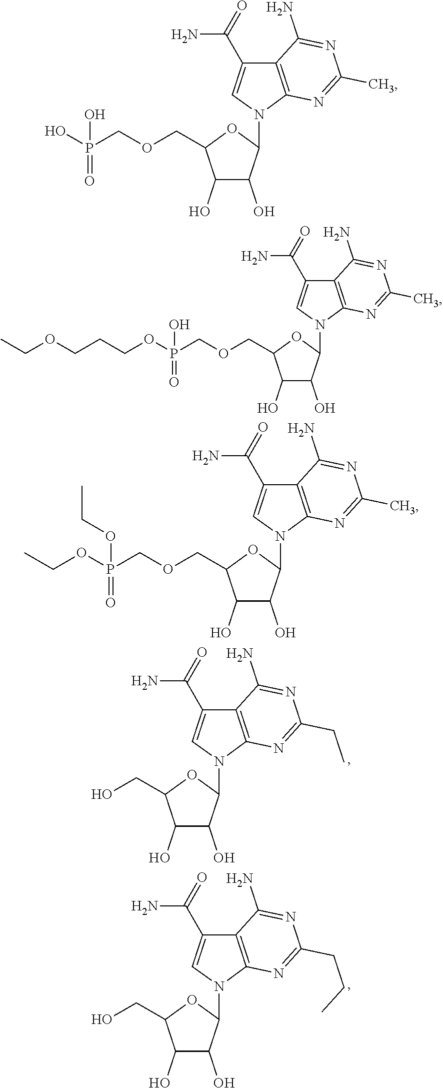

prodrugs thereof, or metabolites thereof.

In one or more embodiments, the present disclosure relates to methods of treating and/or preventing a SARS-CoV-2 infection and/or a SARS-CoV-2 infection-associated disease or disorder (e.g. COVID-19) with one or more compounds of:

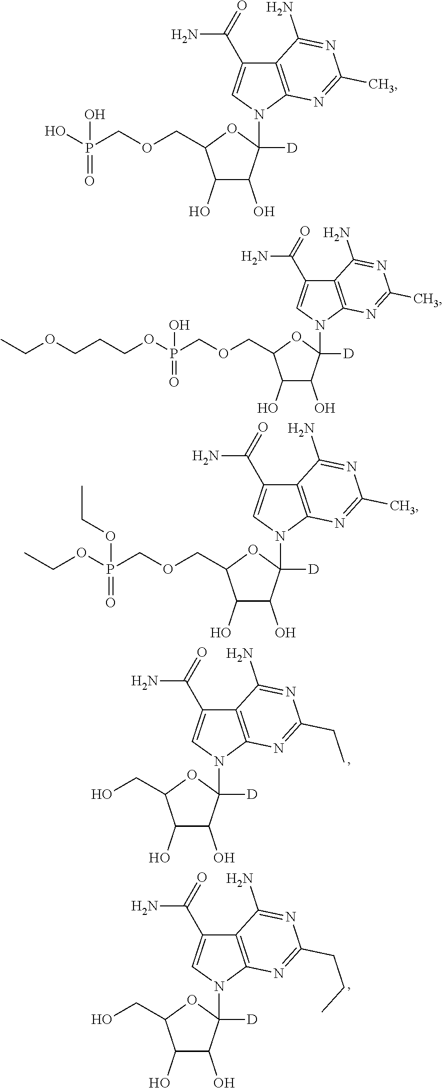

prodrugs thereof, or metabolites thereof.

In one or more embodiments, the present disclosure relates to methods of treating and/or preventing a SARS-CoV-2 infection and/or a SARS-CoV-2 infection-associated disease or disorder (e.g. COVID-19) with one or more compounds of:

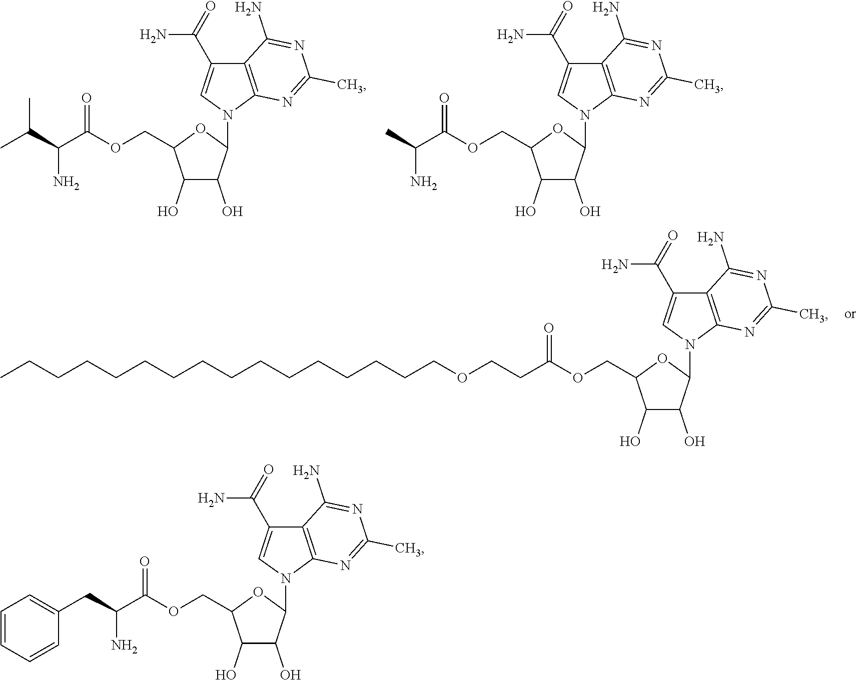 [SMILES: Cc1nc(N)c2c(C(N)=O)cn(C3OC(COC(=O)[C@@H](N)C(C)C)C(O)C3O)c2n1 ; Cc1nc(N)c2c(C(N)=O)cn(C3OC(COC(=O)[C@H](C)N)C(O)C3O)c2n1 ; CCCCCCCCCCCCCCCCOCCC(=O)OCC1OC(n2cc(C(N)=O)c3c(N)nc(C)nc32)C(O)C1O ; Cc1nc(N)c2c(C(N)=O)cn(C3OC(COC(=O)[C@@H](N)Cc4ccccc4)C(O)C3O)c2n1]

a prodrug thereof, or a metabolite thereof.

In one or more embodiments, the present disclosure relates to methods of treating and/or preventing a SARS-CoV-2 infection and/or a SARS-CoV-2 infection-associated disease or disorder (e.g. COVID-19) with one or more compounds of:

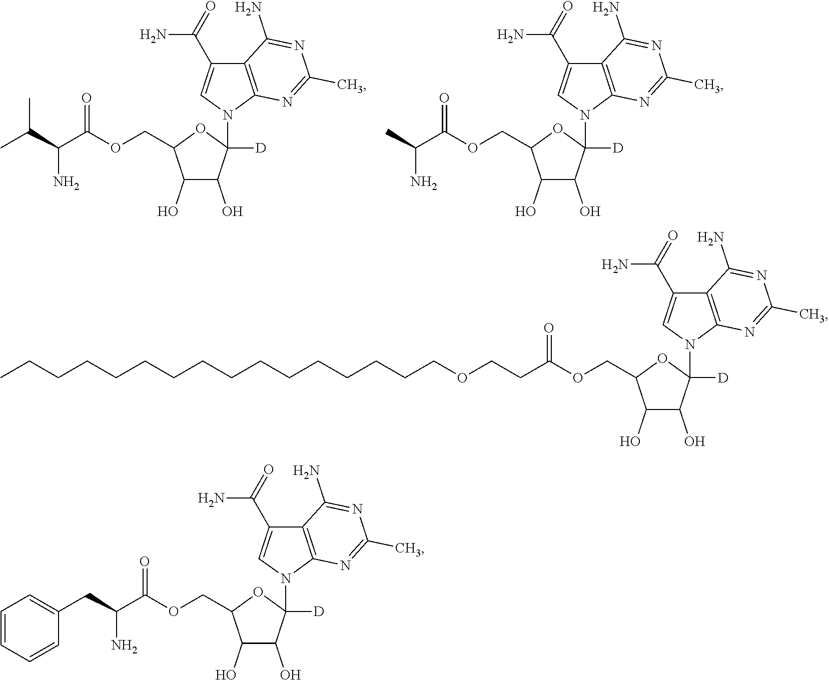 [SMILES: [2H]C1(n2cc(C(N)=O)c3c(N)nc(C)nc32)OC(COC(=O)[C@@H](N)C(C)C)C(O)C1O ; [2H]C1(n2cc(C(N)=O)c3c(N)nc(C)nc32)OC(COC(=O)[C@H](C)N)C(O)C1O ; [2H]C1(n2cc(C(N)=O)c3c(N)nc(C)nc32)OC(COC(=O)CCOCCCCCCCCCCCCCCCC)C(O)C1O ; [2H]C1(n2cc(C(N)=O)c3c(N)nc(C)nc32)OC(COC(=O)[C@@H](N)Cc2ccccc2)C(O)C1O]

prodrugs thereof, or metabolites thereof.

In some embodiments, the present disclosure relates to methods of treating and/or preventing a SARS-CoV-2 infection and/or a SARS-CoV-2 infection-associated disease or disorder (e.g. COVID-19) with one or more compounds of 1, 2, 3, 4, 5, 6, 71, 77, 76, 107, 111, 126, 133, 137, 139, 141, 143, or 145, or any combination thereof.

In some embodiments, the present disclosure relates to methods of treating and/or preventing a SARS-CoV-2 infection and/or a SARS-CoV-2 infection-associated disease or disorder (e.g. COVID-19) with one or more compounds selected from the compounds described in Table 1, prodrugs thereof, and metabolites thereof.

In some embodiments, the present disclosure relates to methods of treating and/or preventing a SARS-CoV-2 infection and/or a SARS-CoV-2 infection-associated disease or disorder (e.g. COVID-19) with one or more compounds selected from the prodrugs of the compounds described in Table 1.

In some embodiments, the present disclosure relates to methods of treating and/or preventing a SARS-CoV-2 infection and/or a SARS-CoV-2 infection-associated disease or disorder (e.g. COVID-19) with one or more compounds selected from the metabolites of the compounds described in Table 1.

In some embodiments, the present disclosure relates to methods of treating and/or preventing a SARS-CoV-2 infection and/or a SARS-CoV-2 infection-associated disease or disorder (e.g. COVID-19) with one or more compounds selected from the compounds described in Table 1.

In some embodiments, the present disclosure relates to methods of treating and/or preventing a SARS-CoV-2 infection and/or a SARS-CoV-2 infection-associated disease or disorder (e.g. COVID-19) with Compound 1, a prodrug thereof, or a metabolite thereof.

In some embodiments, the present disclosure relates to methods of treating and/or preventing a SARS-CoV-2 infection and/or a SARS-CoV-2 infection-associated disease or disorder (e.g. COVID-19) with a prodrug of Compound 1.

In some embodiments, the present disclosure relates to methods of treating and/or preventing a SARS-CoV-2 infection and/or a SARS-CoV-2 infection-associated disease or disorder (e.g. COVID-19) with a metabolite of Compound 1.

In some embodiments, the present disclosure relates to methods of treating and/or preventing a SARS-CoV-2 infection and/or a SARS-CoV-2 infection-associated disease or disorder (e.g. COVID-19) with Compound 1.

TABLE 1
Compounds ID Number and Chemical Structures

| ID Number | Compound Chemical Structure |
|---|---|
| 1 | 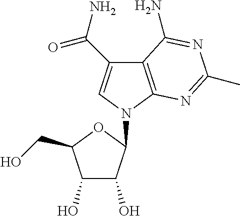 [SMILES: Cc1nc(N)c2c(C(N)=O)cn([C@@H]3O[C@H](CO)[C@@H](O)[C@H]3O)c2n1] |
| 2 | 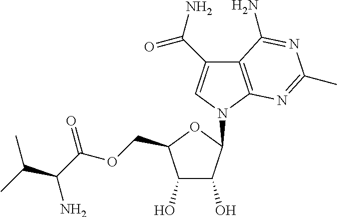 [SMILES: Cc1nc(N)c2c(C(N)=O)cn([C@@H]3O[C@H](COC(=O)[C@@H](N)C(C)C)[C@@H](O)[C@H]3O)c2n1] |
| 3 | 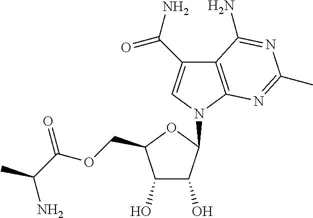 [SMILES: Cc1nc(N)c2c(C(N)=O)cn([C@@H]3O[C@H](COC(=O)[C@H](C)N)[C@@H](O)[C@H]3O)c2n1] |
| 4 | 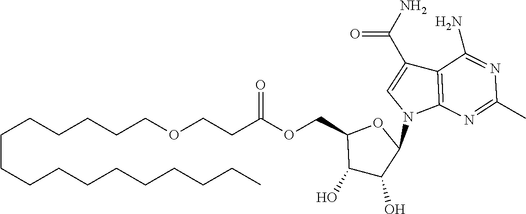 [SMILES: CCCCCCCCCCCCCCCCOCCC(=O)OC[C@H]1O[C@@H](n2cc(C(N)=O)c3c(N)nc(C)nc32)[C@H](O)[C@@H]1O] |
| 5 | 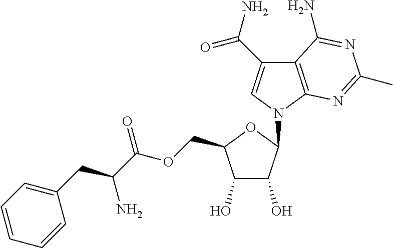 [SMILES: Cc1nc(N)c2c(C(N)=O)cn([C@@H]3O[C@H](COC(=O)[C@@H](N)Cc4ccccc4)[C@@H](O)[C@H]3O)c2n1] |
| 6 | 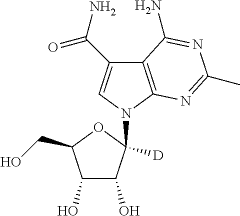 [SMILES: [2H][C@@]1(n2cc(C(N)=O)c3c(N)nc(C)nc32)O[C@H](CO)[C@@H](O)[C@H]1O] |
| 71 | 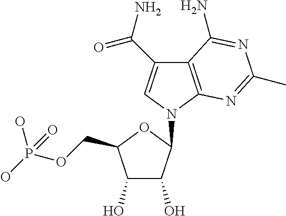 [SMILES: Cc1nc(N)c2c(C(N)=O)cn([C@@H]3O[C@H](COP(=O)(O)O)[C@@H](O)[C@H]3O)c2n1] |
| 77 | 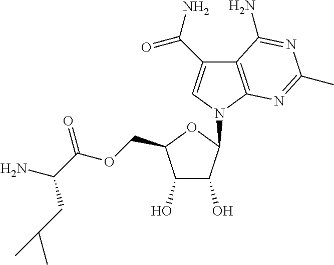 [SMILES: Cc1nc(N)c2c(C(N)=O)cn([C@@H]3O[C@H](COC(=O)[C@@H](N)CC(C)C)[C@@H](O)[C@H]3O)c2n1] |
| 76 | 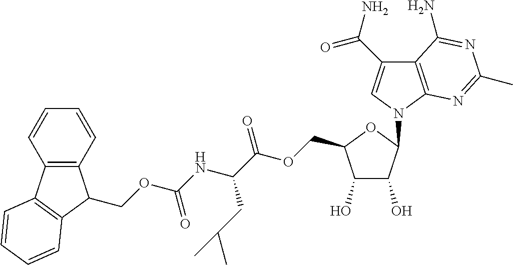 [SMILES: Cc1nc(N)c2c(C(N)=O)cn([C@@H]3O[C@H](COC(=O)[C@H](CC(C)C)NC(=O)OCC4c5ccccc5-c5ccccc54)[C@@H](O)[C@H]3O)c2n1] |
| 107 | 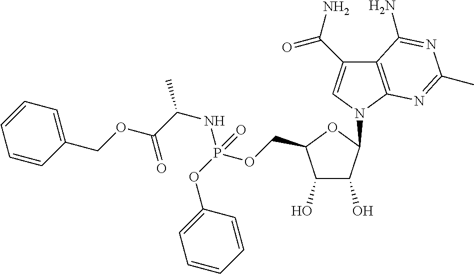 [SMILES: Cc1nc(N)c2c(C(N)=O)cn([C@@H]3O[C@H](COP(=O)(N[C@@H](C)C(=O)OCc4ccccc4)Oc4ccccc4)[C@@H](O)[C@H]3O)c2n1] |
| 111 | 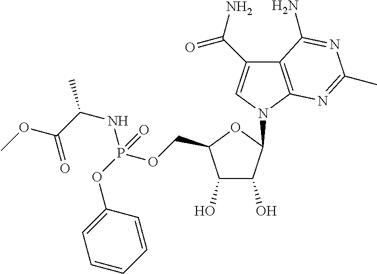 |
| 126 | 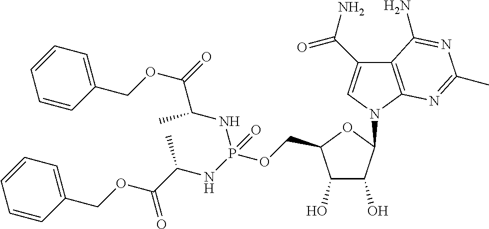 [SMILES: Cc1nc(N)c2c(C(N)=O)cn([C@@H]3O[C@H](COP(=O)(N[C@@H](C)C(=O)OCc4ccccc4)N[C@H](C)C(=O)OCc4ccccc4)[C@@H](O)[C@H]3O)c2n1] |
| 133 | 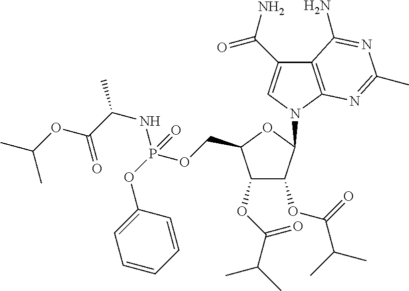 [SMILES: Cc1nc(N)c2c(C(N)=O)cn([C@@H]3O[C@H](COP(=O)(N[C@@H](C)C(=O)OC(C)C)Oc4ccccc4)[C@@H](OC(=O)C(C)C)[C@H]3OC(=O)C(C)C)c2n1] |
| 137 | 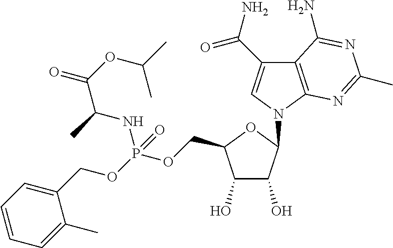 [SMILES: Cc1nc(N)c2c(C(N)=O)cn([C@@H]3O[C@H](COP(=O)(N[C@@H](C)C(=O)OC(C)C)OCc4ccccc4C)[C@@H](O)[C@H]3O)c2n1] |
| 139 | 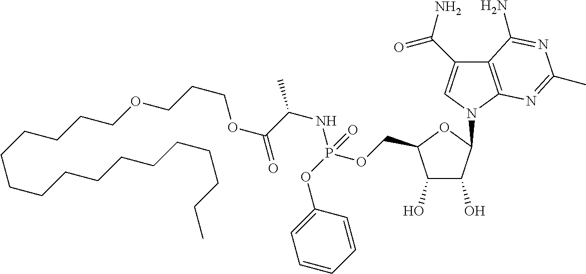 [SMILES: CCCCCCCCCCCCCCCCOCCCOC(=O)[C@H](C)NP(=O)(OC[C@H]1O[C@@H](n2cc(C(N)=O)c3c(N)nc(C)nc32)[C@H](O)[C@@H]1O)Oc1ccccc1] |
| 141 | 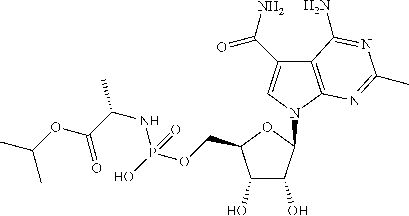 [SMILES: Cc1nc(N)c2c(C(N)=O)cn([C@@H]3O[C@H](COP(=O)(O)N[C@@H](C)C(=O)OC(C)C)[C@@H](O)[C@H]3O)c2n1] |
| 143 | 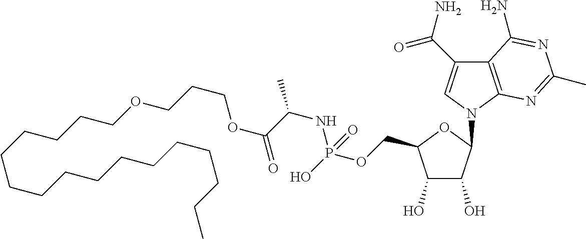 [SMILES: CCCCCCCCCCCCCCCCOCCCOC(=O)[C@H](C)NP(=O)(O)OC[C@H]1O[C@@H](n2cc(C(N)=O)c3c(N)nc(C)nc32)[C@H](O)[C@@H]1O] |
| 145 | 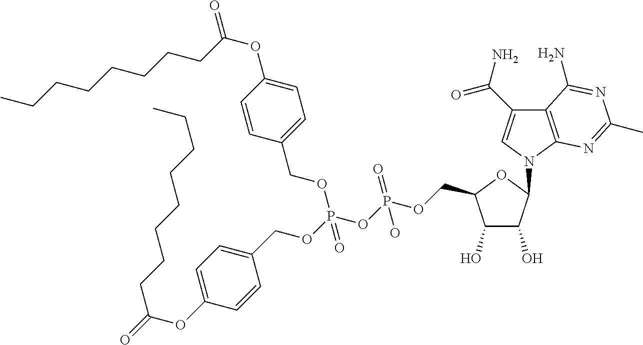 [SMILES: CCCCCCCCC(=O)Oc1ccc(COP(=O)(OCc2ccc(OC(=O)CCCCCCCC)cc2)OP(=O)(O)OC[C@H]2O[C@@H](n3cc(C(N)=O)c4c(N)nc(C)nc43)[C@H](O)[C@@H]2O)cc1] |
| 7 | 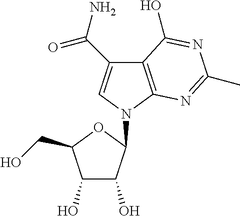 [SMILES: Cc1nc(O)c2c(C(N)=O)cn([C@@H]3O[C@H](CO)[C@@H](O)[C@H]3O)c2n1] |
| 8 | 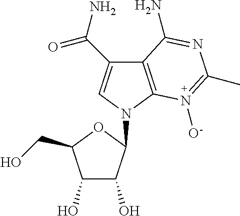 |
| 10 | 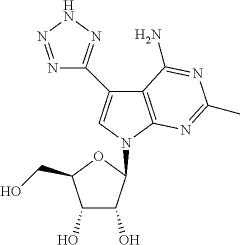 [SMILES: Cc1nc(N)c2c(-c3nn[nH]n3)cn([C@@H]3O[C@H](CO)[C@@H](O)[C@H]3O)c2n1] |
| 11 | 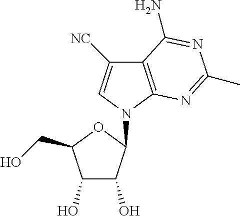 [SMILES: Cc1nc(N)c2c(C#N)cn([C@@H]3O[C@H](CO)[C@@H](O)[C@H]3O)c2n1] |
| 12 | 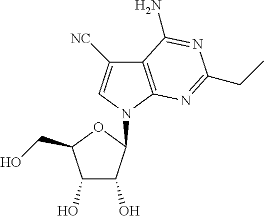 [SMILES: CCc1nc(N)c2c(C#N)cn([C@@H]3O[C@H](CO)[C@@H](O)[C@H]3O)c2n1] |
| 14 | 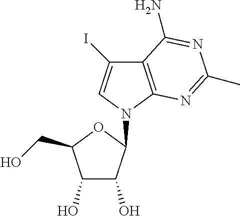 [SMILES: Cc1nc(N)c2c(I)cn([C@@H]3O[C@H](CO)[C@@H](O)[C@H]3O)c2n1] |
| 16 | 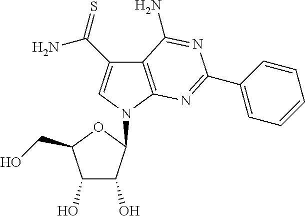 [SMILES: NC(=S)c1cn([C@@H]2O[C@H](CO)[C@@H](O)[C@H]2O)c2nc(-c3ccccc3)nc(N)c12] |
| 17 | 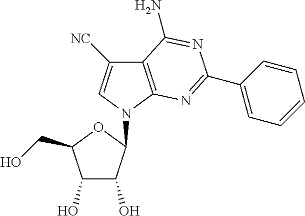 [SMILES: N#Cc1cn([C@@H]2O[C@H](CO)[C@@H](O)[C@H]2O)c2nc(-c3ccccc3)nc(N)c12] |
| 18 | 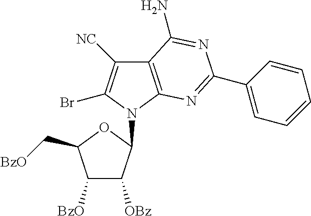 |
| 19 | 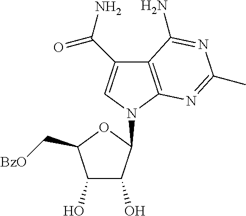 |
| 20 | 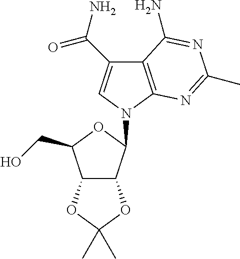 [SMILES: Cc1nc(N)c2c(C(N)=O)cn([C@@H]3O[C@H](CO)[C@H]4OC(C)(C)O[C@H]43)c2n1] |
| 21 | 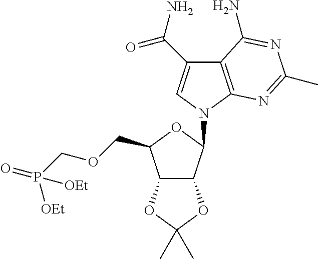 [SMILES: CCOP(=O)(COC[C@H]1O[C@@H](n2cc(C(N)=O)c3c(N)nc(C)nc32)[C@@H]2OC(C)(C)O[C@@H]21)OCC] |
| 22 | 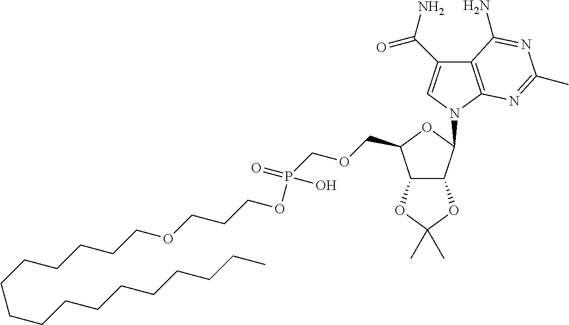 [SMILES: CCCCCCCCCCCCCCCCOCCCOP(=O)(O)COC[C@H]1O[C@@H](n2cc(C(N)=O)c3c(N)nc(C)nc32)[C@@H]2OC(C)(C)O[C@@H]21] |
| 27 | 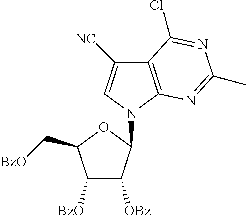 |
| 28 | 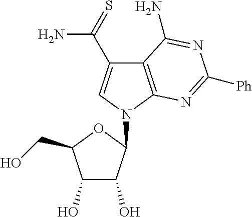 [SMILES: NC(=S)c1cn([C@@H]2O[C@H](CO)[C@@H](O)[C@H]2O)c2nc(-c3ccccc3)nc(N)c12] |
| 29 | 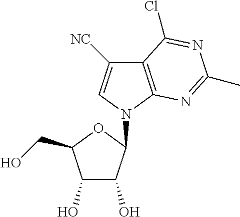 [SMILES: Cc1nc(Cl)c2c(C#N)cn([C@@H]3O[C@H](CO)[C@@H](O)[C@H]3O)c2n1] |
| 30 | 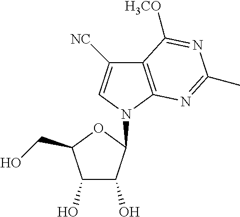 [SMILES: COc1nc(C)nc2c1c(C#N)cn2[C@@H]1O[C@H](CO)[C@@H](O)[C@H]1O] |
| 31 | 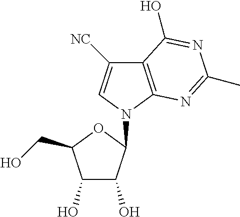 [SMILES: Cc1nc(O)c2c(C#N)cn([C@@H]3O[C@H](CO)[C@@H](O)[C@H]3O)c2n1] |
| 32 | 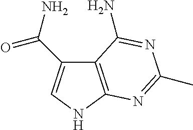 |
| 33 | 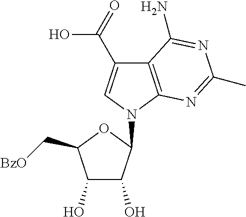 |
| 34 | 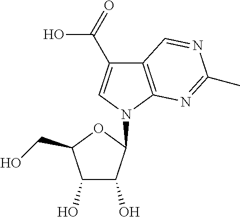 [SMILES: Cc1ncc2c(C(=O)O)cn([C@@H]3O[C@H](CO)[C@@H](O)[C@H]3O)c2n1] |
| 35 | 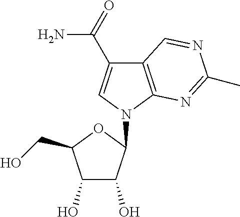 [SMILES: Cc1ncc2c(C(N)=O)cn([C@@H]3O[C@H](CO)[C@@H](O)[C@H]3O)c2n1] |
| 36 | 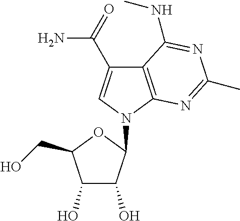 [SMILES: CNc1nc(C)nc2c1c(C(N)=O)cn2[C@@H]1O[C@H](CO)[C@@H](O)[C@H]1O] |
| 37 | 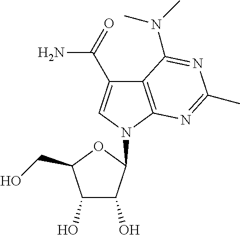 [SMILES: Cc1nc(N(C)C)c2c(C(N)=O)cn([C@@H]3O[C@H](CO)[C@@H](O)[C@H]3O)c2n1] |
| 38 | 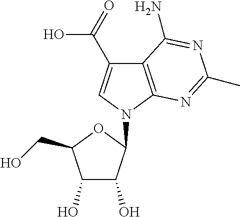 [SMILES: Cc1nc(N)c2c(C(=O)O)cn([C@@H]3O[C@H](CO)[C@@H](O)[C@H]3O)c2n1] |
| 39 | 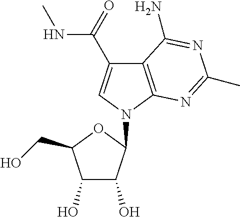 [SMILES: CNC(=O)c1cn([C@@H]2O[C@H](CO)[C@@H](O)[C@H]2O)c2nc(C)nc(N)c12] |
| 41 | 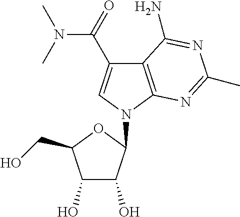 [SMILES: Cc1nc(N)c2c(C(=O)N(C)C)cn([C@@H]3O[C@H](CO)[C@@H](O)[C@H]3O)c2n1] |
| 42 | 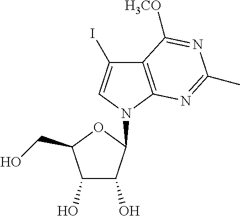 [SMILES: COc1nc(C)nc2c1c(I)cn2[C@@H]1O[C@H](CO)[C@@H](O)[C@H]1O] |
| 43 | 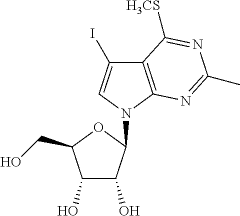 |
| 44 | 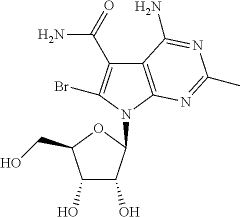 [SMILES: Cc1nc(N)c2c(C(N)=O)c(Br)n([C@@H]3O[C@H](CO)[C@@H](O)[C@H]3O)c2n1] |
| 45 | 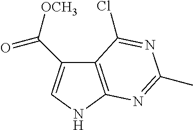 |
| 46 | 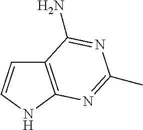 |
| 47 | 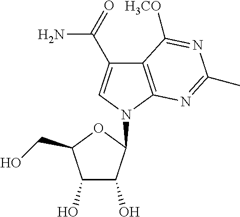 [SMILES: COc1nc(C)nc2c1c(C(N)=O)cn2[C@@H]1O[C@H](CO)[C@@H](O)[C@H]1O] |
| 48 | 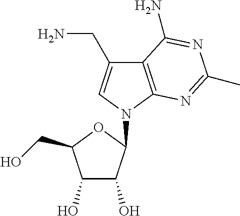 [SMILES: Cc1nc(N)c2c(CN)cn([C@@H]3O[C@H](CO)[C@@H](O)[C@H]3O)c2n1] |
| 49 | 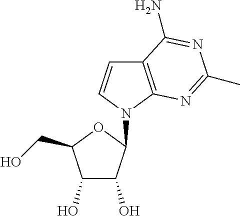 [SMILES: Cc1nc(N)c2ccn([C@@H]3O[C@H](CO)[C@@H](O)[C@H]3O)c2n1] |
| 50 | 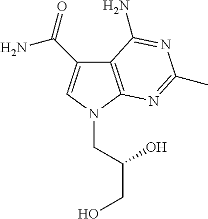 [SMILES: Cc1nc(N)c2c(C(N)=O)cn(C[C@H](O)CO)c2n1] |
| 51 | 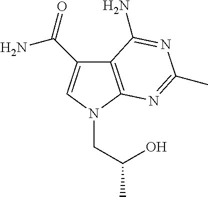 [SMILES: Cc1nc(N)c2c(C(N)=O)cn(C[C@@H](C)O)c2n1] |
| 54 | 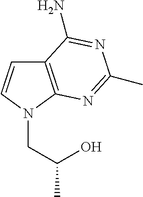 |
| 61 | 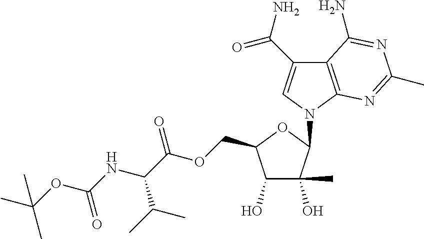 [SMILES: Cc1nc(N)c2c(C(N)=O)cn([C@@H]3O[C@H](COC(=O)[C@@H](NC(=O)OC(C)(C)C)C(C)C)[C@@H](O)[C@@]3(C)O)c2n1] |
| 63 | 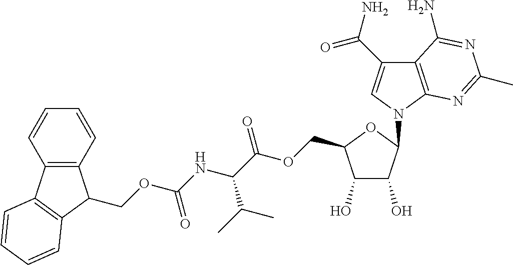 [SMILES: Cc1nc(N)c2c(C(N)=O)cn([C@@H]3O[C@H](COC(=O)[C@@H](NC(=O)OCC4c5ccccc5-c5ccccc54)C(C)C)[C@@H](O)[C@H]3O)c2n1] |
| 65 | 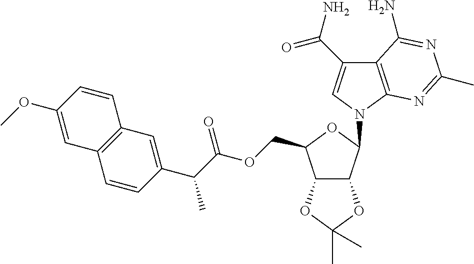 |
| 66 | 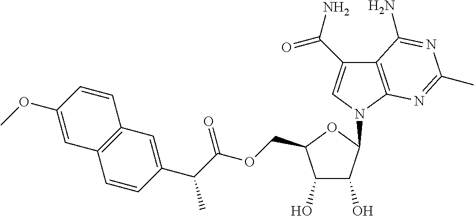 |
| 68 | 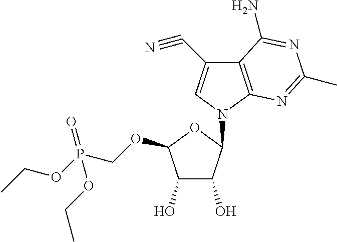 [SMILES: CCOP(=O)(CO[C@H]1O[C@@H](n2cc(C#N)c3c(N)nc(C)nc32)[C@H](O)[C@@H]1O)OCC] |
| 69 | 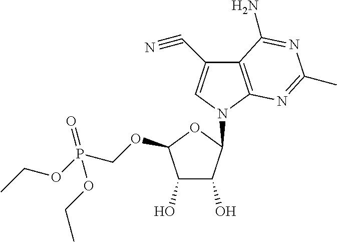 [SMILES: CCOP(=O)(CO[C@H]1O[C@@H](n2cc(C#N)c3c(N)nc(C)nc32)[C@H](O)[C@@H]1O)OCC] |
| 72 | 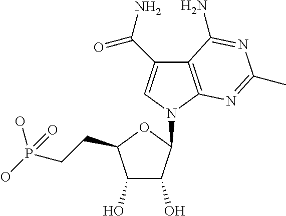 [SMILES: Cc1nc(N)c2c(C(N)=O)cn([C@@H]3O[C@H](CCP(=O)(O)O)[C@@H](O)[C@H]3O)c2n1] |
| 73 | 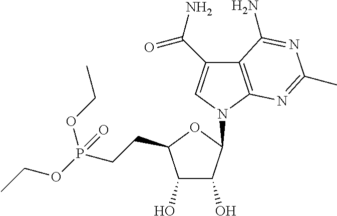 [SMILES: CCOP(=O)(CC[C@H]1O[C@@H](n2cc(C(N)=O)c3c(N)nc(C)nc32)[C@H](O)[C@@H]1O)OCC] |
| 78 | 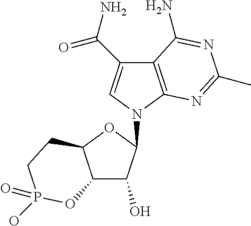 |
| 81 | 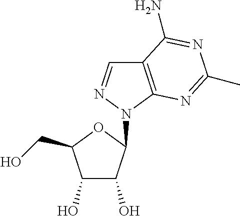 [SMILES: Cc1nc(N)c2cnn([C@@H]3O[C@H](CO)[C@@H](O)[C@H]3O)c2n1] |
| 82 | 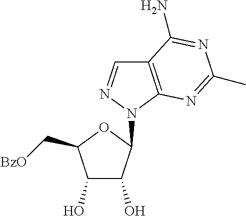 |
| 83 | 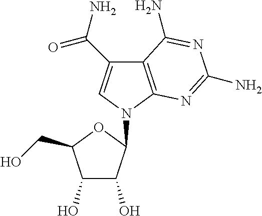 [SMILES: NC(=O)c1cn([C@@H]2O[C@H](CO)[C@@H](O)[C@H]2O)c2nc(N)nc(N)c12] |
| 84 | 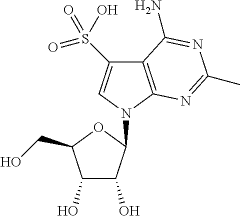 [SMILES: Cc1nc(N)c2c(S(=O)(=O)O)cn([C@@H]3O[C@H](CO)[C@@H](O)[C@H]3O)c2n1] |
| 85 | 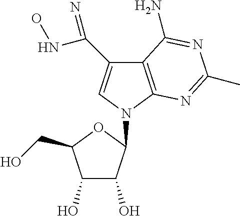 [SMILES: Cc1nc(N)c2c(C(=N)NO)cn([C@@H]3O[C@H](CO)[C@@H](O)[C@H]3O)c2n1] |
| 86 | 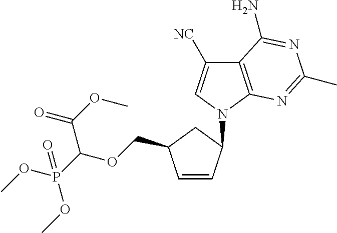 |
| 88 | 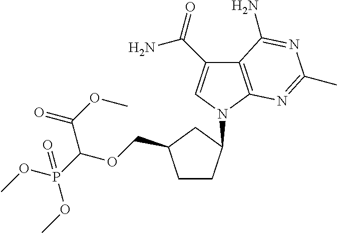 |
| 91 | 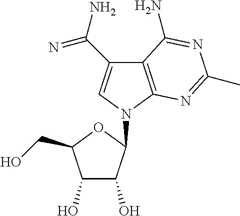 [SMILES: Cc1nc(N)c2c(C(=N)N)cn([C@@H]3O[C@H](CO)[C@@H](O)[C@H]3O)c2n1] |
| 93 | 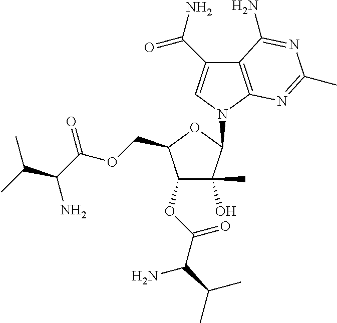 |
| 94 | 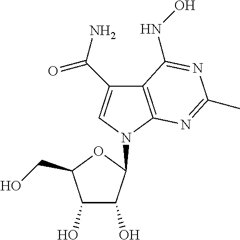 [SMILES: Cc1nc(NO)c2c(C(N)=O)cn([C@@H]3O[C@H](CO)[C@@H](O)[C@H]3O)c2n1] |
| 95 | 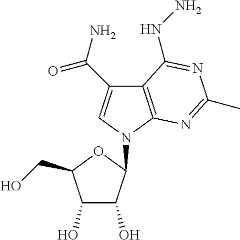 [SMILES: Cc1nc(NN)c2c(C(N)=O)cn([C@@H]3O[C@H](CO)[C@@H](O)[C@H]3O)c2n1] |
| 96 | 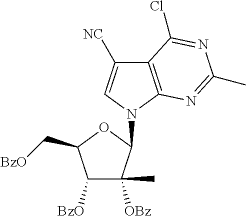 |
| 98 | 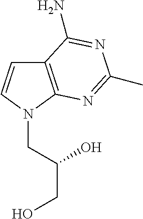 [SMILES: Cc1nc(N)c2ccn(C[C@H](O)CO)c2n1] |
| 100 | 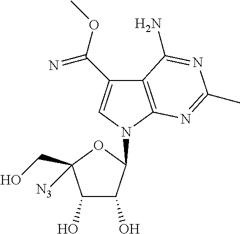 [SMILES: COC(=N)c1cn([C@@H]2O[C@@](CO)(N=[N+]=[N-])[C@@H](O)[C@H]2O)c2nc(C)nc(N)c12] |
| 103 | 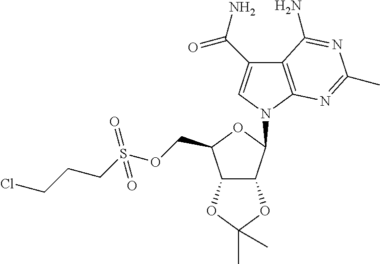 [SMILES: Cc1nc(N)c2c(C(N)=O)cn([C@@H]3O[C@H](COS(=O)(=O)CCCCl)[C@H]4OC(C)(C)O[C@H]43)c2n1] |
| 104 | 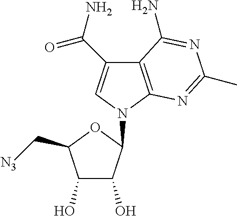 [SMILES: Cc1nc(N)c2c(C(N)=O)cn([C@@H]3O[C@H](CN=[N+]=[N-])[C@@H](O)[C@H]3O)c2n1] |
| 106 | 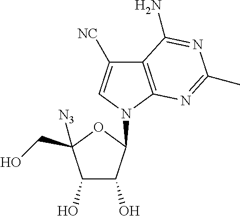 [SMILES: Cc1nc(N)c2c(C#N)cn([C@@H]3O[C@@](CO)(N=[N+]=[N-])[C@@H](O)[C@H]3O)c2n1] |
| 108 | 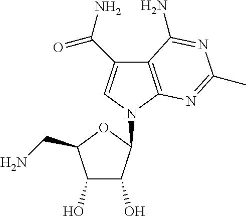 [SMILES: Cc1nc(N)c2c(C(N)=O)cn([C@@H]3O[C@H](CN)[C@@H](O)[C@H]3O)c2n1] |
| 110 | 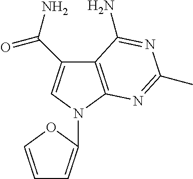 [SMILES: Cc1nc(N)c2c(C(N)=O)cn(-c3ccco3)c2n1] |
| 114 | 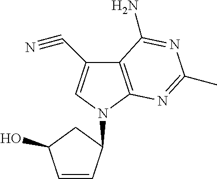 [SMILES: Cc1nc(N)c2c(C#N)cn([C@H]3C=C[C@@H](O)C3)c2n1] |
| 115 | 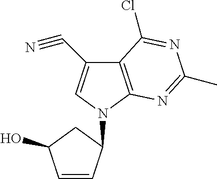 [SMILES: Cc1nc(Cl)c2c(C#N)cn([C@H]3C=C[C@@H](O)C3)c2n1] |
| 116 | 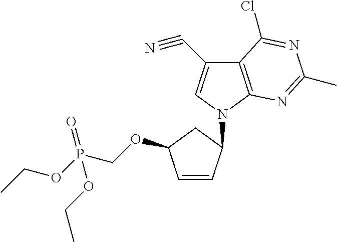 [SMILES: CCOP(=O)(CO[C@@H]1C=C[C@H](n2cc(C#N)c3c(Cl)nc(C)nc32)C1)OCC] |
| 117 | 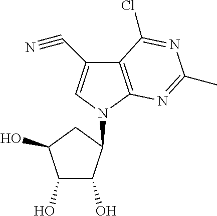 [SMILES: Cc1nc(Cl)c2c(C#N)cn([C@@H]3C[C@H](O)[C@@H](O)[C@H]3O)c2n1] |
| 120 | 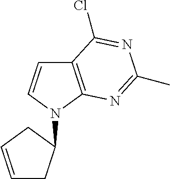 |
| 121 | 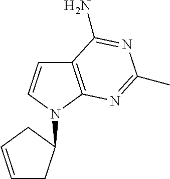 |
| 122 | 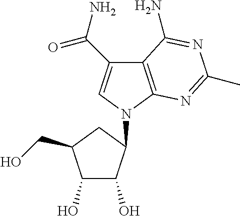 [SMILES: Cc1nc(N)c2c(C(N)=O)cn([C@@H]3C[C@H](CO)[C@@H](O)[C@H]3O)c2n1] |
| 123 | 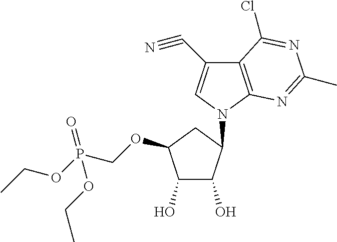 [SMILES: CCOP(=O)(CO[C@H]1C[C@@H](n2cc(C#N)c3c(Cl)nc(C)nc32)[C@H](O)[C@@H]1O)OCC] |
| 124 | 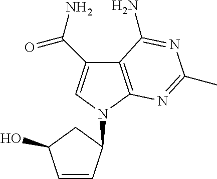 [SMILES: Cc1nc(N)c2c(C(N)=O)cn([C@H]3C=C[C@@H](O)C3)c2n1] |
| 125 | 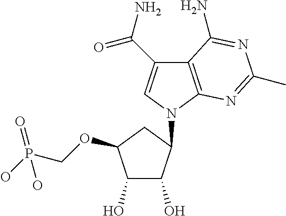 [SMILES: Cc1nc(N)c2c(C(N)=O)cn([C@@H]3C[C@H](OCP(=O)(O)O)[C@@H](O)[C@H]3O)c2n1] |
| 128 | 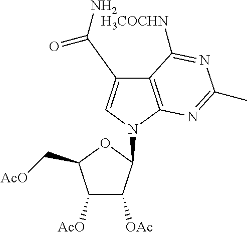 |
| 129 | 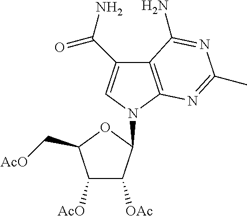 [SMILES: CC(=O)OC[C@H]1O[C@@H](n2cc(C(N)=O)c3c(N)nc(C)nc32)[C@H](OC(C)=O)[C@@H]1OC(C)=O] |
| 130 | 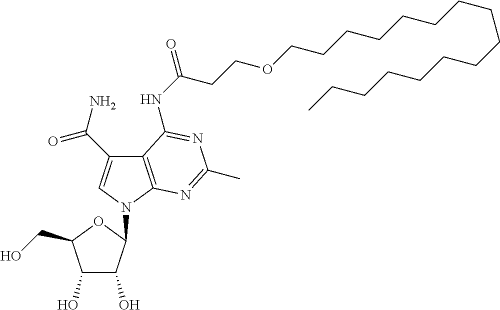 [SMILES: CCCCCCCCCCCCCCCCOCCC(=O)Nc1nc(C)nc2c1c(C(N)=O)cn2[C@@H]1O[C@H](CO)[C@@H](O)[C@H]1O] |
| 131 | 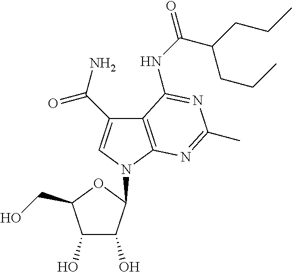 [SMILES: CCCC(CCC)C(=O)Nc1nc(C)nc2c1c(C(N)=O)cn2[C@@H]1O[C@H](CO)[C@@H](O)[C@H]1O] |
| 132 | 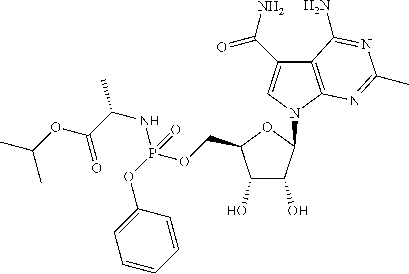 [SMILES: Cc1nc(N)c2c(C(N)=O)cn([C@@H]3O[C@H](COP(=O)(N[C@@H](C)C(=O)OC(C)C)Oc4ccccc4)[C@@H](O)[C@H]3O)c2n1] |
| 134 | 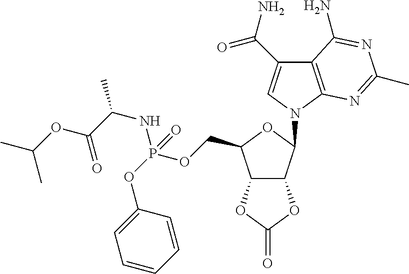 [SMILES: Cc1nc(N)c2c(C(N)=O)cn([C@@H]3O[C@H](COP(=O)(N[C@@H](C)C(=O)OC(C)C)Oc4ccccc4)[C@H]4OC(=O)O[C@H]43)c2n1] |
| 136 | 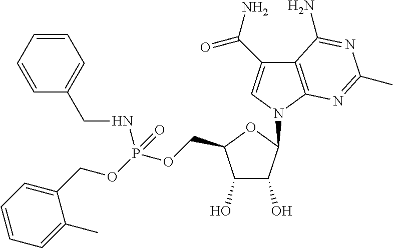 [SMILES: Cc1nc(N)c2c(C(N)=O)cn([C@@H]3O[C@H](COP(=O)(NCc4ccccc4)OCc4ccccc4C)[C@@H](O)[C@H]3O)c2n1] |
| 138 | 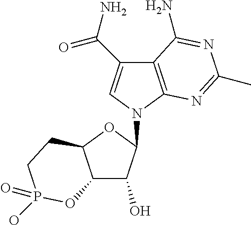 |
| 140 | 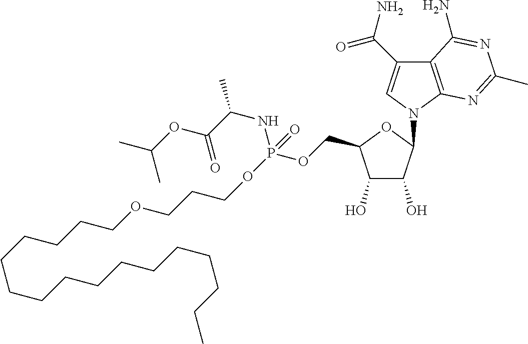 [SMILES: CCCCCCCCCCCCCCCCOCCCOP(=O)(N[C@@H](C)C(=O)OC(C)C)OC[C@H]1O[C@@H](n2cc(C(N)=O)c3c(N)nc(C)nc32)[C@H](O)[C@@H]1O] |
| 142 | 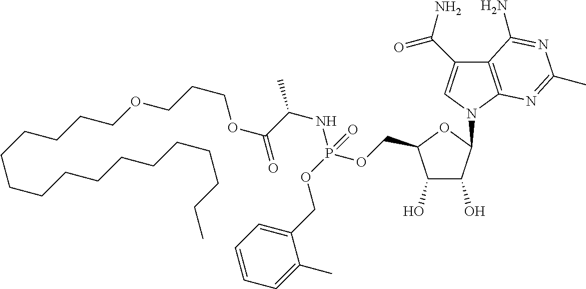 [SMILES: CCCCCCCCCCCCCCCCOCCCOC(=O)[C@H](C)NP(=O)(OCc1ccccc1C)OC[C@H]1O[C@@H](n2cc(C(N)=O)c3c(N)nc(C)nc32)[C@H](O)[C@@H]1O] |
| 144 | 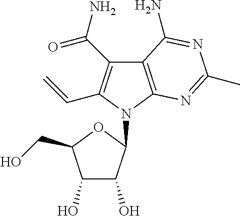 [SMILES: C=Cc1c(C(N)=O)c2c(N)nc(C)nc2n1[C@@H]1O[C@H](CO)[C@@H](O)[C@H]1O] |
| 9 | 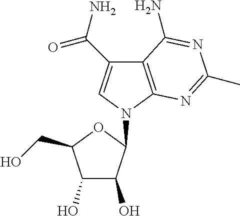 [SMILES: Cc1nc(N)c2c(C(N)=O)cn([C@@H]3O[C@H](CO)[C@@H](O)[C@@H]3O)c2n1] |
| 13 | 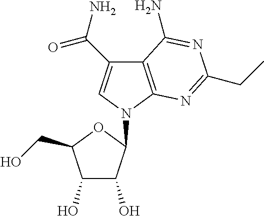 [SMILES: CCc1nc(N)c2c(C(N)=O)cn([C@@H]3O[C@H](CO)[C@@H](O)[C@H]3O)c2n1] |
| 15 | 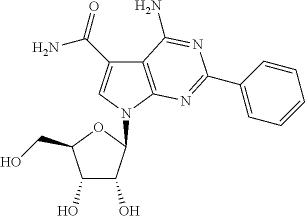 [SMILES: NC(=O)c1cn([C@@H]2O[C@H](CO)[C@@H](O)[C@H]2O)c2nc(-c3ccccc3)nc(N)c12] |
| 23 | 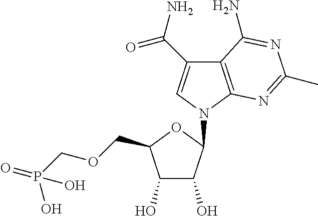 [SMILES: Cc1nc(N)c2c(C(N)=O)cn([C@@H]3O[C@H](COCP(=O)(O)O)[C@@H](O)[C@H]3O)c2n1] |
| 24 | 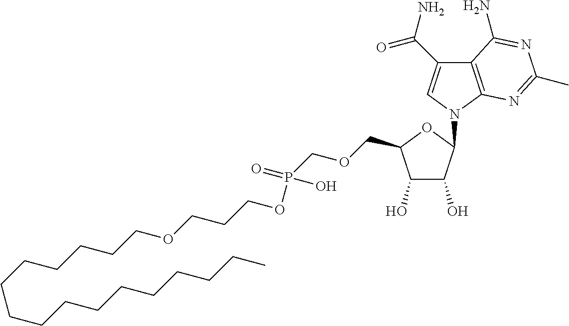 [SMILES: CCCCCCCCCCCCCCCCOCCCOP(=O)(O)COC[C@H]1O[C@@H](n2cc(C(N)=O)c3c(N)nc(C)nc32)[C@H](O)[C@@H]1O] |
| 25 | 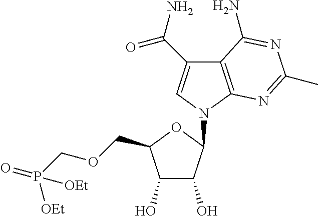 [SMILES: CCOP(=O)(COC[C@H]1O[C@@H](n2cc(C(N)=O)c3c(N)nc(C)nc32)[C@H](O)[C@@H]1O)OCC] |
| 26 | 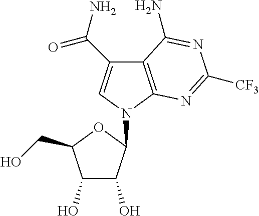 [SMILES: NC(=O)c1cn([C@@H]2O[C@H](CO)[C@@H](O)[C@H]2O)c2nc(C(F)(F)F)nc(N)c12] |
| 40 | 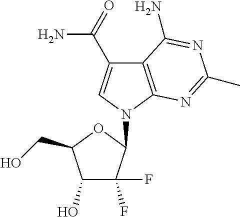 |
| 52 | 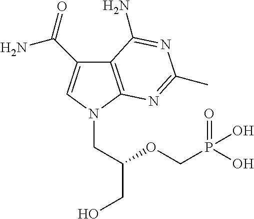 [SMILES: Cc1nc(N)c2c(C(N)=O)cn(C[C@@H](CO)OCP(=O)(O)O)c2n1] |
| 53 | 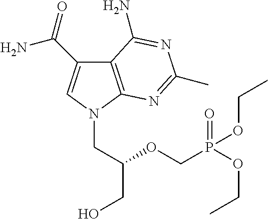 [SMILES: CCOP(=O)(CO[C@H](CO)Cn1cc(C(N)=O)c2c(N)nc(C)nc21)OCC] |
| 55 | 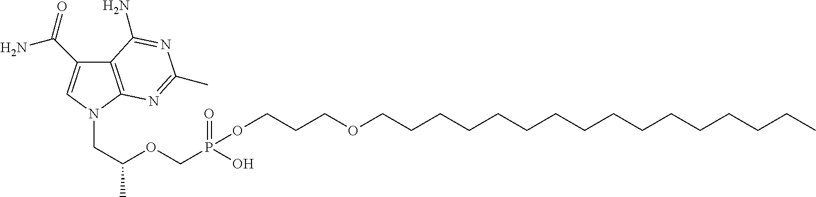 [SMILES: CCCCCCCCCCCCCCCCOCCCOP(=O)(O)CO[C@H](C)Cn1cc(C(N)=O)c2c(N)nc(C)nc21] |
| 56 | 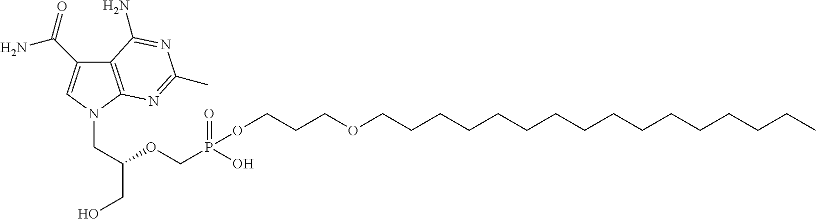 [SMILES: CCCCCCCCCCCCCCCCOCCCOP(=O)(O)CO[C@H](CO)Cn1cc(C(N)=O)c2c(N)nc(C)nc21] |
| 57 | 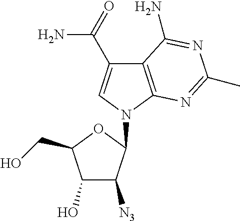 [SMILES: Cc1nc(N)c2c(C(N)=O)cn([C@@H]3O[C@H](CO)[C@@H](O)[C@@H]3N=[N+]=[N-])c2n1] |
| 58 | 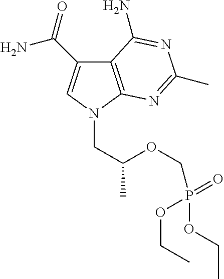 [SMILES: CCOP(=O)(CO[C@H](C)Cn1cc(C(N)=O)c2c(N)nc(C)nc21)OCC] |
| 59 | 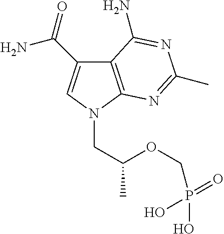 [SMILES: Cc1nc(N)c2c(C(N)=O)cn(C[C@@H](C)OCP(=O)(O)O)c2n1] |
| 60 | 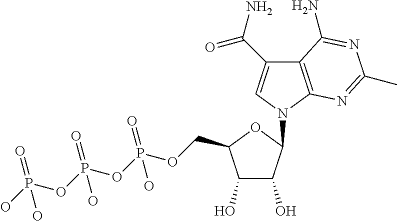 [SMILES: Cc1nc(N)c2c(C(N)=O)cn([C@@H]3O[C@H](COP(=O)(O)OP(=O)(O)OP(=O)(O)O)[C@@H](O)[C@H]3O)c2n1] |
| 62 | 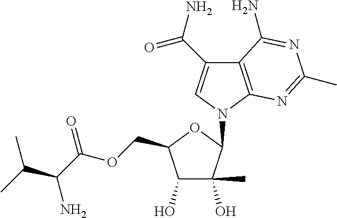 [SMILES: Cc1nc(N)c2c(C(N)=O)cn([C@@H]3O[C@H](COC(=O)[C@@H](N)C(C)C)[C@@H](O)[C@@]3(C)O)c2n1] |
| 64 | 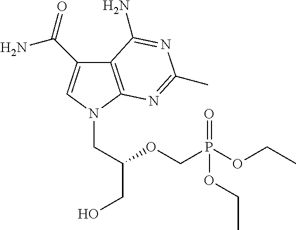 [SMILES: CCOP(=O)(CO[C@H](CO)Cn1cc(C(N)=O)c2c(N)nc(C)nc21)OCC] |
| 67 | 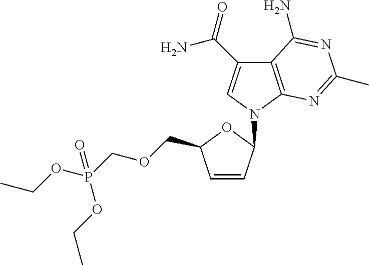 [SMILES: CCOP(=O)(COC[C@@H]1C=C[C@H](n2cc(C(N)=O)c3c(N)nc(C)nc32)O1)OCC] |
| 70 | 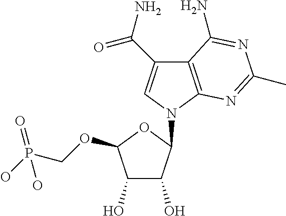 [SMILES: Cc1nc(N)c2c(C(N)=O)cn([C@@H]3O[C@H](OCP(=O)(O)O)[C@@H](O)[C@H]3O)c2n1] |
| 74 | 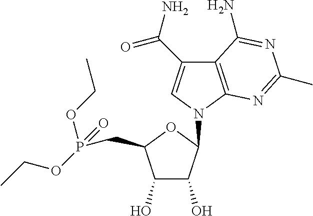 [SMILES: CCOP(=O)(C[C@H]1O[C@@H](n2cc(C(N)=O)c3c(N)nc(C)nc32)[C@H](O)[C@@H]1O)OCC] |
| 75 | 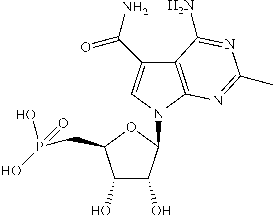 [SMILES: Cc1nc(N)c2c(C(N)=O)cn([C@@H]3O[C@H](CP(=O)(O)O)[C@@H](O)[C@H]3O)c2n1] |
| 79 | 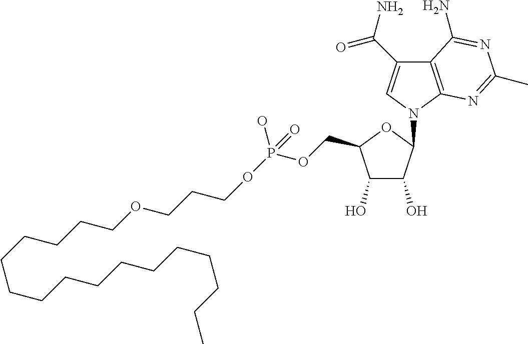 [SMILES: CCCCCCCCCCCCCCCCOCCCOP(=O)(O)OC[C@H]1O[C@@H](n2cc(C(N)=O)c3c(N)nc(C)nc32)[C@H](O)[C@@H]1O] |
| 80 | 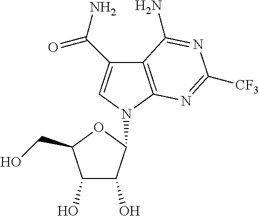 [SMILES: NC(=O)c1cn([C@H]2O[C@H](CO)[C@@H](O)[C@H]2O)c2nc(C(F)(F)F)nc(N)c12] |
| 87 | 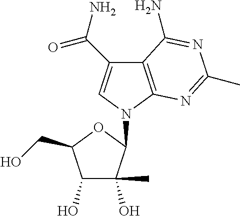 [SMILES: Cc1nc(N)c2c(C(N)=O)cn([C@@H]3O[C@H](CO)[C@@H](O)[C@@]3(C)O)c2n1] |
| 89 | 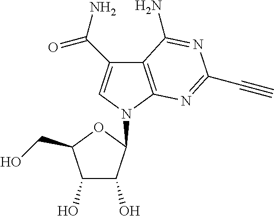 [SMILES: C#Cc1nc(N)c2c(C(N)=O)cn([C@@H]3O[C@H](CO)[C@@H](O)[C@H]3O)c2n1] |
| 90 | 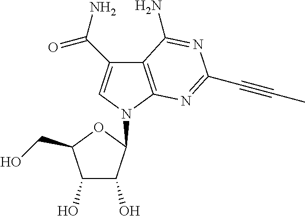 [SMILES: CC#Cc1nc(N)c2c(C(N)=O)cn([C@@H]3O[C@H](CO)[C@@H](O)[C@H]3O)c2n1] |
| 92 | 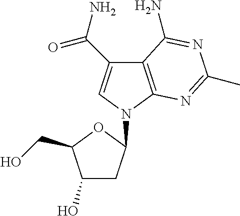 [SMILES: Cc1nc(N)c2c(C(N)=O)cn([C@H]3C[C@H](O)[C@@H](CO)O3)c2n1] |
| 97 | 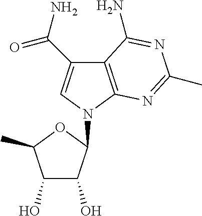 |
| 99 | 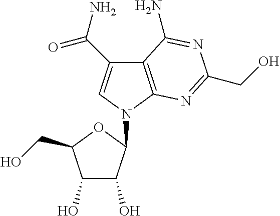 [SMILES: NC(=O)c1cn([C@@H]2O[C@H](CO)[C@@H](O)[C@H]2O)c2nc(CO)nc(N)c12] |
| 101 | 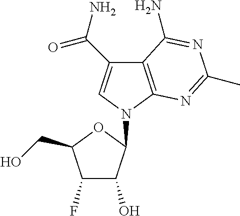 [SMILES: Cc1nc(N)c2c(C(N)=O)cn([C@@H]3O[C@H](CO)[C@@H](F)[C@H]3O)c2n1] |
| 102 | 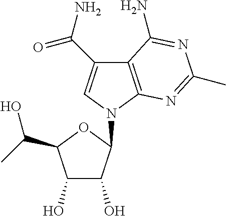 [SMILES: Cc1nc(N)c2c(C(N)=O)cn([C@@H]3O[C@H](C(C)O)[C@@H](O)[C@H]3O)c2n1] |
| 105 | 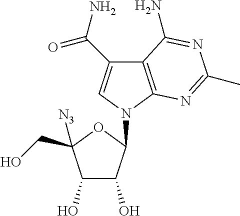 [SMILES: Cc1nc(N)c2c(C(N)=O)cn([C@@H]3O[C@@](CO)(N=[N+]=[N-])[C@@H](O)[C@H]3O)c2n1] |
| 109 | 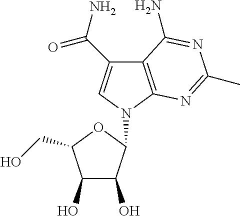 [SMILES: Cc1nc(N)c2c(C(N)=O)cn([C@H]3O[C@@H](CO)[C@H](O)[C@@H]3O)c2n1] |
| 112 | 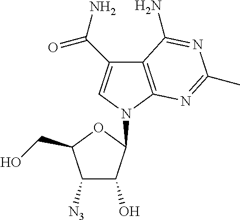 [SMILES: Cc1nc(N)c2c(C(N)=O)cn([C@@H]3O[C@H](CO)[C@@H](N=[N+]=[N-])[C@H]3O)c2n1] |
| 113 | 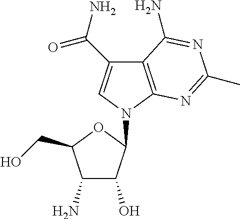 [SMILES: Cc1nc(N)c2c(C(N)=O)cn([C@@H]3O[C@H](CO)[C@@H](N)[C@H]3O)c2n1] |
| 118 | 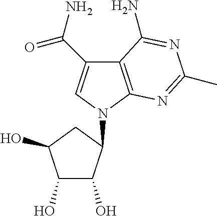 [SMILES: Cc1nc(N)c2c(C(N)=O)cn([C@@H]3C[C@H](O)[C@@H](O)[C@H]3O)c2n1] |
| 119 | 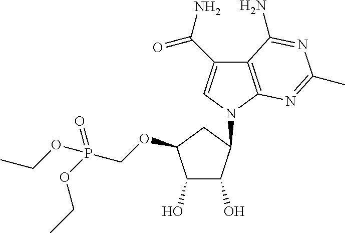 [SMILES: CCOP(=O)(CO[C@H]1C[C@@H](n2cc(C(N)=O)c3c(N)nc(C)nc32)[C@H](O)[C@@H]1O)OCC] |
| 135 | 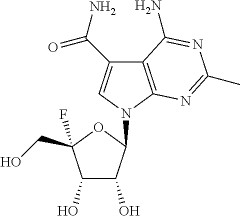 [SMILES: Cc1nc(N)c2c(C(N)=O)cn([C@@H]3O[C@](F)(CO)[C@@H](O)[C@H]3O)c2n1] |
| 127 | 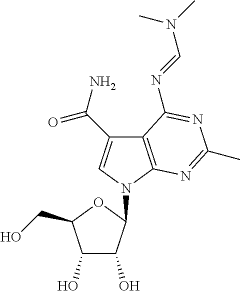 [SMILES: Cc1nc(/N=C/N(C)C)c2c(C(N)=O)cn([C@@H]3O[C@H](CO)[C@@H](O)[C@H]3O)c2n1] |

In some aspects, the present disclosure is directed to the use of a compound of the present disclosure for preparing a medicament for preventing a SARS-CoV-2 infection or a SARS-CoV-2-infection associated disease or disorder (e.g. COVID-19) in a patient in need thereof.

In some aspects, the present disclosure is directed to compounds for use preventing a SARS-CoV-2 infection or a SARS-CoV-2-infection associated disease or disorder (e.g. COVID-19) in a patient in need thereof.

In some aspects, the present disclosure is directed to methods for treating or preventing a SARS-CoV-2 infection or a SARS-CoV-2-infection associated disease or disorder (e.g. COVID-19) in a patient in need thereof, comprising administering a compound described herein, or a pharmaceutically acceptable salt, solvate, enantiomer, diastereomer, racemate or mixture thereof. In some aspects, the present disclosure is directed to methods for treating or preventing a SARS-CoV-2 infection or a SARS-CoV-2-infection associated disease or disorder (e.g. COVID-19) in a patient in need thereof, comprising administering a compound described herein, or a pharmaceutically acceptable salt or solvate thereof.

In some aspects, the present disclosure is directed to methods for treating or preventing a SARS-CoV-2 infection or a SARS-CoV-2-infection associated disease or disorder (e.g. COVID-19) in a patient in need thereof, comprising administering a compound described herein, or a pharmaceutically acceptable salt, solvate, enantiomer, diastereomer, racemate or mixture thereof, wherein said administration results in the in vivo formation of one or more active metabolites.

### Methods of Synthesis

The compounds of the present disclosure may be made by a variety of methods, including standard chemistry. Suitable synthetic routes are depicted in the schemes given below and according to the methods as described in PCT/US2016/046056.

The compounds described herein (e.g., compounds of Formula I, Formula IA, Formula IB or Formula II) may be prepared by methods known in the art of organic synthesis as set forth in part by the following synthetic schemes and examples. In the schemes described below, it is well understood that protecting groups for sensitive or reactive groups are employed where necessary in accordance with general principles or chemistry. Protecting groups are manipulated according to standard methods of organic synthesis. These groups are removed at a convenient stage of the compound synthesis using methods that are readily apparent to those skilled in the art. The selection processes, as well as the reaction conditions and order of their execution, shall be consistent with the preparation of compounds of Formula I, Formula IA, Formula IB or Formula II.

Those skilled in the art will recognize if a stereocenter exists in the compounds of Formula I, Formula IA, Formula IB or Formula II. Accordingly, the present disclosure includes both possible stereoisomers (unless specified in the synthesis) and includes not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art.

The compounds described herein may be made from commercially available starting materials or synthesized using known organic, inorganic, and/or enzymatic processes.

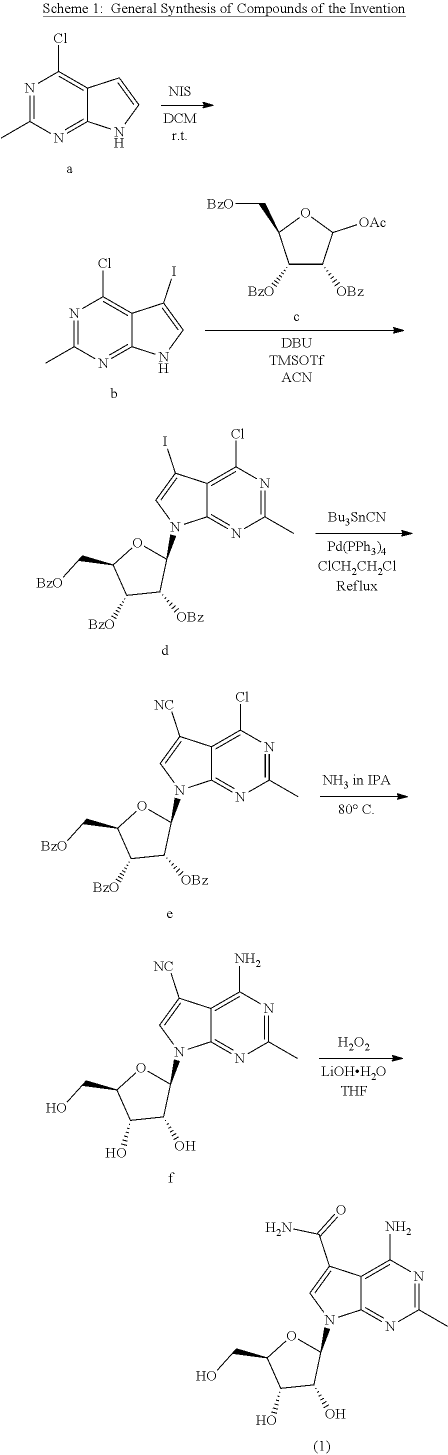

As shown above in Scheme 1, 4-chloro-2-methyl-7H-pyrrolo[2,3-d]pyrimidine (a; Scheme 1 numbering) can be iodinated in the presence of N-iodosuccinimide (NIS). The resulting 4-chloro-5-iodo-2-methyl-7H-pyrrolo[2,3-d]pyrimidine (b) can be treated with a protected furan (c) as shown in Step 2 to give compound (d). Radical substitution of (d) gives the corresponding cyano derivative (e) which can undergo deprotection and nucleophilic aromatic substitution at the chlorine-bound carbon to give amine-derivative (f). Finally, nitrile hydration of (f) gives 4-amino-7-((2R,3R,4S,5R)-3,4-dihydroxy-5-(hydroxymethyl)tetrahydrofuran-2-yl)-2-methyl-7H-pyrrolo[2,3-d]pyrimidine-5-carboxamide (1).

In certain embodiments, the compounds of the disclosure, e.g., Formula I, Formula IA, Formula IB or Formula II may be prepared as enantiomers, diastereomers, and racemates. In some embodiments, compounds of the disclosure, e.g., Formula I, Formula IA, Formula IB or Formula II include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the disclosure. Unless otherwise stated, all tautomeric forms of the compounds disclosed herein are within the scope of the disclosure.

Compounds of the disclosure, e.g., Formula I, Formula IA, Formula IB or Formula II can be synthesized substantially free of impurities. Compounds of the disclosure are more than or equal to about 99% w/w pure. In certain embodiments the phosphonate esters may be prepared on a large scale, for example on an industrial production scale rather than on an experimental/laboratory scale. For example, a batch-type process according to the methods of the disclosure allows the preparation of batches of at least 1 g, or at least 5 g, or at least 10 g, or at least 100 g, or at least 1 kg, or at least 100 kg of phosphonate ester product. Furthermore, the methods allow the preparation of a phosphonate ester product having a purity of at least 98%, or at least 98.5% as measured by HPLC. In preferred embodiments, these products are obtained in a reaction sequence that does not involve purification by any form of chromatography (e.g., gas chromatography, HPLC, preparative LC, size exclusion chromatography, and the like).

### Morphic Forms

In an embodiment, the disclosure is directed methods of treating and/or preventing a SARS-CoV-2 infection and/or a SARS-CoV-2 infection-associated disease or disorder (e.g., COVID-19) with Compound 1. In some embodiments, the disclosure is directed methods of treating and/or preventing a SARS-CoV-2 infection and/or a SARS-CoV-2 infection-associated disease or disorder (e.g., COVID-19) with an amorphic form of Compound 1. In some embodiments, the disclosure is directed methods of treating and/or preventing a SARS-CoV-2 infection and/or a SARS-CoV-2 infection-associated disease or disorder (e.g., COVID-19) with a polymorphic form of Compound 1 selected from Form A, Form B, Form C, Form D, Form E, Form F, and Form G. In some embodiments, the disclosure is directed methods of treating and/or preventing a SARS-CoV-2 infection and/or a SARS-CoV-2 infection-associated disease or disorder (e.g., COVID-19) with one or more of the morphic forms of Compound 1 as described in PCT/US2018/052180, which is incorporated herein by reference in its entirety.

### Form A

In some embodiments, the disclosure is directed methods of treating and/or preventing a SARS-CoV-2 infection and/or a SARS-CoV-2 infection-associated disease or disorder (e.g. COVID-19) with Compound 1 Form A.

Morphic Form A can be characterized by a PXRD peak at about 26.2 °2θ (e.g., 26.2±0.2 °2θ; 26.2±0.1 °2θ; or 26.2±0.0 °2θ; Cu Kα1 radiation). Form A can further be characterized by PXRD peaks at about 23.2 °2θ and/or 26.5 °2θ (e.g., ±0.2 °2θ, 0.1 °2θ, or ±0.0 °2θ; Cu Kα1 radiation). Form A can be further characterized by PXRD peaks at about 10.7 °2θ, 11.6 °2θ, 12.2 °2θ, 15.3 °2θ, and/or 18.6 °2θ (e.g., ±0.2 °2θ, 0.1 °2θ, or ±0.0 °2θ; Cu Kα1 radiation).

In some embodiments, Form A can be characterized by PXRD peaks at about 26.2 °2θ and about 23.2 °2θ (e.g., ±0.2 °2θ, 0.1 °2θ, or ±0.0 °2θ; Cu Kα1 radiation). In some embodiments, Form A can be characterized by PXRD peaks at about 26.2 °2θ and about 26.5 °2θ (e.g., ±0.2 °2θ, 0.1 °2θ, or ±0.0 °2θ; Cu Kα1 radiation). In some embodiments, Form A can be characterized by PXRD peaks at about 26.2 °2θ, 23.2 °2θ, and about 26.5 °2θ (e.g., ±0.2 °2θ, 0.1 °2θ, or ±0.0 °2θ; Cu Kα1 radiation).

In some embodiments, Form A can be characterized by PXRD peaks at about 10.7 °2θ, about 26.2 °2θ, about 23.2 °2θ, and about 26.5 °2θ (e.g., ±0.2 °2θ, 0.1 °2θ, or ±0.0 °2θ; Cu Kα1 radiation). In some embodiments, Form A can be characterized by PXRD peaks at about 11.6 °2θ, about 26.2 °2θ, about 23.2 °2θ, and about 26.5 °2θ (e.g., ±0.2 °2θ, 0.1 °2θ, or ±0.0 °2θ; Cu Kα1 radiation). In some embodiments, Form A can be characterized by PXRD peaks at about 12.2 °2θ, about 26.2 °2θ, about 23.2 °2θ, and about 26.5 °2θ (e.g., ±0.2 °2θ, 0.1 °2θ, or ±0.0 °2θ; Cu Kα1 radiation). In some embodiments, Form A can be characterized by PXRD peaks at about 15.3 °2θ, about 26.2 °2θ, about 23.2 °2θ, and about 26.5 °2θ (e.g., ±0.2 °2θ, 0.1 °2θ, or ±0.0 °2θ; Cu Kα1 radiation). In some embodiments, Form A can be characterized by PXRD peaks at about 18.6 °2θ, about 26.2 °2θ, about 23.2 °2θ, and about 26.5 °2θ (e.g., ±0.2 °2θ, 0.1 °2θ, or ±0.0 °2θ; Cu Kα1 radiation).

In some embodiments, Form A is characterized by PXRD peaks at about 15.9 °2θ and about 23.8 °2θ (e.g., ±0.2 °2θ, 0.1 °2θ, or ±0.0 °2θ; Cu Kα1 radiation).

In some embodiments, Form A is characterized by PXRD peaks at about 10.7 °2θ, about 11.6 °2θ, and about 12.2 °2θ (e.g., ±0.2 °2θ, 0.1 °2θ, or ±0.0 °2θ; Cu Kα1 radiation). In some embodiments, Form A is characterized by PXRD peaks at about 10.7 °2θ, about 11.6 °2θ, about 12.2 °2θ, and about 15.3 °2θ (e.g., ±0.2 °2θ, 0.1 °2θ, or ±0.0 °2θ; Cu Kα1 radiation). In some embodiments, Form A is characterized by a single peak at about 11.6 °2θ (e.g., ±0.2 °2θ, 0.1 °2θ, or ±0.0 °2θ; Cu Kα1 radiation).

In some embodiments, Form A is characterized by PXRD peaks at about 10.7 °2θ, about 11.6 °2θ, about 12.2 °2θ, about 15.3 °2θ, and about 15.9 °2θ (e.g., ±0.2 °2θ, 0.1 °2θ, or ±0.0 °2θ; Cu Kα1 radiation). In some embodiments, Form A is characterized by PXRD peaks at about 10.7 °2θ, about 11.6 °2θ, about 12.2 °2θ, about 15.3 °2θ, and about 16.3 °2θ (e.g., ±0.2 °2θ, 0.1 °2θ, or ±0.0 °2θ; Cu Kα1 radiation). In some embodiments, Form A is characterized by PXRD peaks at about 10.7 °2θ, about 11.6 °2θ, about 12.2 °2θ, about 15.3 °2θ, about 15.9 °2θ and about 16.3 °2θ (e.g., ±0.2 °2θ, 0.1 °2θ, or ±0.0 °2θ; Cu Kα1 radiation). In some embodiments, Form A is characterized by PXRD peaks at about 10.7 °2θ, about 11.6 °2θ, about 12.2 °2θ, about 15.3 °2θ, and about 18.6 °2θ (e.g., ±0.2 °2θ, ±0.1 °2θ, or ±0.0 °2θ; Cu Kα1 radiation). In some embodiments, Form A is characterized by PXRD peaks at about 10.7 °2θ, about 11.6 °2θ, about 12.2 °2θ, about 15.3 °2θ, about 15.9 °2θ, about 16.3 °2θ and about 18.6 °2θ (e.g., ±0.2 °2θ, 0.1 °2θ, or ±0.0 °2θ; Cu Kα1 radiation). In some embodiments, Form A is characterized by PXRD peaks at about 10.7 °2θ, about 11.6 °2θ, about 12.2 °2θ, about 15.3 °2θ, about 15.9 °2θ, and about 18.6 °2θ (e.g., ±0.2 °2θ, 0.1 °2θ, or ±0.0 °2θ; Cu Kα1 radiation). In some embodiments, Form A is characterized by PXRD peaks at about 10.7 °2θ, about 11.6 °2θ, about 12.2 °2θ, about 15.3 °2θ, about 16.3 °2θ and about 18.6 °2θ (e.g., ±0.2 °2θ, 0.1 °2θ, or ±0.0 °2θ; Cu Kα1 radiation).

In some embodiments, Form A can be characterized by PXRD peaks at about 10.7 °2θ, about 23.2 °2θ, about 26.2 °2θ, and about 26.5 °2θ (e.g., ±0.2 °2θ, 0.1 °2θ, or ±0.0 °2θ; Cu Kα1 radiation). In some embodiments, Form A can be characterized by PXRD peaks at about 10.7 °2θ, about 11.6 °2θ, about 23.2 °2θ, about 26.2 °2θ, and about 26.5 °2θ (e.g., ±0.2 °2θ, 0.1 °2θ, or ±0.0 °2θ; Cu Kα1 radiation). In some embodiments, Form A can be characterized by PXRD peaks at about 10.7 °2θ, about 11.6 °2θ, about 12.2 °2θ, about 23.2 °2θ, about 26.2 °2θ, and about 26.5 °2θ (e.g., ±0.2 °2θ, 0.1 °2θ, or ±0.0 °2θ; Cu Kα1 radiation). In some embodiments, Form A can be characterized by PXRD peaks at about 10.7 °2θ, about 11.6 °2θ, about 12.2 °2θ, about 15.3 °2θ, about 23.2 °2θ, about 26.2 °2θ, and about 26.5 °2θ (e.g., ±0.2 °2θ, 0.1 °2θ, or ±0.0 °2θ; Cu Kα1 radiation). In some embodiments, Form A can be characterized by PXRD peaks at about 10.7 °2θ, about 11.6 °2θ, about 12.2 °2θ, about 15.3 °2θ, about 18.6 °2θ, about 23.2 °2θ, about 26.2 °2θ, and about 26.5 °2θ (e.g., ±0.2 °2θ, 0.1 °2θ, or ±0.0 °2θ; Cu Kα1 radiation).

Accordingly, in some embodiments, morphic Form A is characterized by one, two, three, four, five, six, seven or eight peaks selected from about 10.7, 11.6, 12.2, 15.3, 18.6, 23.2, 26.2, and 26.5 °2θ (e.g., ±0.2 °2θ, 0.1 °2θ, or ±0.0 °2θ; Cu Kα1 radiation). In some embodiments, morphic Form A is characterized by one peak selected from about 10.7, 11.6, 12.2, 15.3, 18.6, 23.2, 26.2, and 26.5 °2θ (e.g., ±0.2 °2θ, 0.1 °2θ, or ±0.0 °2θ; Cu Kα1 radiation). In some embodiments, morphic Form A is characterized by two peaks selected from about 10.7, 11.6, 12.2, 15.3, 18.6, 23.2, 26.2, and 26.5 °2θ (e.g., ±0.2 °2θ, 0.1 °2θ, or ±0.0 °2θ; Cu Kα1 radiation). In some embodiments, morphic Form A is characterized by three peaks selected from about 10.7, 11.6, 12.2, 15.3, 18.6, 23.2, 26.2, and 26.5 °2θ (e.g., ±0.2 °2θ, ±0.1 °2θ, or ±0.0 °2θ; Cu Kα1 radiation). In some embodiments, morphic Form A is characterized by four peaks selected from about 10.7, 11.6, 12.2, 15.3, 18.6, 23.2, 26.2, and 26.5 °2θ (e.g., ±0.2 °2θ, 0.1 °2θ, or ±0.0 °2θ; Cu Kα1 radiation). In some embodiments, morphic Form A is characterized by five peaks selected from about 10.7, 11.6, 12.2, 15.3, 18.6, 23.2, 26.2, and 26.5 °2θ (e.g., ±0.2 °2θ, 0.1 °2θ, or ±0.0 °2θ; Cu Kα1 radiation). In some embodiments, morphic Form A is characterized by six peaks selected from about 10.7, 11.6, 12.2, 15.3, 18.6, 23.2, 26.2, and 26.5 °2θ (e.g., ±0.2 °2θ, 0.1 °2θ, or ±0.0 °2θ; Cu Kα1 radiation). In some embodiments, morphic Form A is characterized by seven peaks selected from about 10.7, 11.6, 12.2, 15.3, 18.6, 23.2, 26.2, and 26.5 °2θ (e.g., ±0.2 °2θ, 0.1 °2θ, or ±0.0 °2θ; Cu Kα1 radiation). In some embodiments, morphic Form A is characterized by eight peaks selected from about 10.7, 11.6, 12.2, 15.3, 18.6, 23.2, 26.2, and 26.5 °2θ (e.g., ±0.2 °2θ, 0.1 °2θ, or ±0.0 °2θ; Cu Kα1 radiation).

### Form B

In some embodiments, the disclosure is directed methods of treating and/or preventing a SARS-CoV-2 infection and/or a SARS-CoV-2 infection-associated disease or disorder (e.g. COVID-19) with Compound 1 Form B.

Morphic Form B can be characterized by a PXRD peak at about 22.9 °2θ (±0.2 °2θ; ±0.1 °2θ; or ±0.0 °2θ; Cu Kα1 radiation). Form B can further be characterized by PXRD peaks at about 4.9 °2θ, 10.9 °2θ, 13.3 °2θ, 18.4 °2θ, 20.9 °2θ, and/or 26.1 °2θ (±0.2 °2θ; ±0.1 °2θ; or ±0.0 °2θ; Cu Kα1 radiation). Form B can further be characterized by PXRD peaks at about 13.6 °2θ, 15.1 °2θ, 17.5 °2θ, 19.0 °2θ, 19.5 °2θ, 20.0 °2θ, 20.3 °2θ, 20.4 °2θ, 23.3 °2θ, and/or 25.7 °2θ (±0.2 °2θ; ±0.1 °2θ; or ±0.0 °2θ; Cu Kα1 radiation).

In some embodiments, Form B can be characterized by PXRD peaks at about 4.9 °2θ, about 10.9 °2θ, and about 13.3 °2θ (±0.2 °2θ; ±0.1 °2θ; or ±0.0 °2θ; Cu Kα1 radiation). In some embodiments, Form B can be characterized by PXRD peaks at about 10.9 °2θ, about 13.3 °2θ, and about 18.4 °2θ, (±0.2 °2θ; ±0.1 °2θ; or ±0.0 °2θ; Cu Kα1 radiation). In some embodiments, Form B can be characterized by PXRD peaks at about 13.3 °2θ, about 18.4 °2θ, and about 20.9 °2θ, (±0.2 °2θ; ±0.1 °2θ; or ±0.0 °2θ; Cu Kα1 radiation). In some embodiments, Form B can be characterized by PXRD peaks at about 18.4 °2θ, about 20.9 °2θ, and about 22.9 °2θ (±0.2 °2θ; ±0.1 °2θ; or ±0.0 °2θ; Cu Kα1 radiation). In some embodiments, Form B can be characterized by PXRD peaks at about 20.9 °2θ, about 22.9 °2θ, and about 26.1 °2θ, (±0.2 °2θ; ±0.1 °2θ; or ±0.0 °2θ; Cu Kα1 radiation).

In some embodiments, Form B can be characterized by PXRD peaks at about 22.9 °2θ and about 4.9 °2θ (±0.2 °2θ; ±0.1 °2θ; or ±0.0 °2θ; Cu Kα1 radiation). In some embodiments, Form B can be characterized by PXRD peaks at about 22.9 °2θ, about 4.9 °2θ, and about 10.9 °2θ (±0.2 °2θ; ±0.1 °2θ; or ±0.0 °2θ; Cu Kα1 radiation). In some embodiments, Form B can be characterized by PXRD peaks at about 22.9 °2θ, about 4.9 °2θ, about 10.9 °2θ, and about 13.3 °2θ (±0.2 °2θ; ±0.1 °2θ; or ±0.0 °2θ; Cu Kα1 radiation). In some embodiments, Form B can be characterized by PXRD peaks at about 22.9 °2θ, about 4.9 °2θ, about 10.9 °2θ, about 13.3 °2θ, and about 18. 4 °2θ (±0.2 °2θ; ±0.1 °2θ; or ±0.0 °2θ; Cu Kα1 radiation). In some embodiments, Form B can be characterized by PXRD peaks at about 22.9 °2θ, about 4.9 °2θ, about 10.9 °2θ, about 13.3 °2θ, about 18. 4 °2θ, and about 20.9 °2θ (±0.2 °2θ; ±0.1 °2θ; or ±0.0 °2θ; Cu Kα1 radiation). In some embodiments, Form B can be characterized by PXRD peaks at about 22.9 °2θ, about 4.9 °2θ, about 10.9 °2θ, about 13.3 °2θ, about 18. 4 °2θ, about 20.9 °2θ, and about 26.1 °2θ (±0.2 °2θ; ±0.1 °2θ; or ±0.0 °2θ; Cu Kα1 radiation). In some embodiments, morphic Form B can further be characterized by PXRD peaks at about 23.3 and/or 25.7 °2θ (±0.2 °2θ; ±0.1 °2θ; or ±0.0 °2θ; Cu Kα1 radiation).

In some embodiments, Form B can be characterized by PXRD peaks at about 22.9 °2θ, about 4.9 °2θ, about 10.9 °2θ, about 13.3 °2θ, about 13.6 °2θ, about 18. 4 °2θ, about 20.9 °2θ, and about 26.1 °2θ (±0.2 °2θ; ±0.1 °2θ; or ±0.0 °2θ; Cu Kα1 radiation). In some embodiments, Form B can be characterized by PXRD peaks at about 22.9 °2θ, about 4.9 °2θ, about 10.9 °2θ, about 13.3 °2θ, about 13.6 °2θ, about 15.1 °2θ, about 18. 4 °2θ, about 20.9 °2θ, and about 26.1 °2θ (±0.2 °2θ; ±0.1 °2θ; or ±0.0 °2θ; Cu Kα1 radiation). In some embodiments, Form B can be characterized by PXRD peaks at about 22.9 °2θ, about 4.9 °2θ, about 10.9 °2θ, about 13.3 °2θ, about 13.6 °2θ, about 15.1 °2θ, about 17.5 °2θ, about 18. 4 °2θ, about 20.9 °2θ, and about 26.1 °2θ (±0.2 °2θ; ±0.1 °2θ; or ±0.0 °2θ; Cu Kα1 radiation). In some embodiments, Form B can be characterized by PXRD peaks at about 22.9 °2θ, about 4.9 °2θ, about 10.9 °2θ, about 13.3 °2θ, about 13.6 °2θ, about 15.1 °2θ, about 17.5 °2θ, about 18. 4 °2θ, about 19.0 °2θ, about 20.9 °2θ, and about 26.1 °2θ (±0.2 °2θ; ±0.1 °2θ; or ±0.0 °2θ; Cu Kα1 radiation). In some embodiments, Form B can be characterized by PXRD peaks at about 22.9 °2θ, about 4.9 °2θ, about 10.9 °2θ, about 13.3 °2θ, about 13.6 °2θ, about 15.1 °2θ, about 17.5 °2θ, about 18. 4 °2θ, about 19.0 °2θ, about 19.5 °2θ, about 20.9 °2θ, and about 26.1 °2θ (±0.2 °2θ; ±0.1 °2θ; or ±0.0 °2θ; Cu Kα1 radiation). In some embodiments, Form B can be characterized by PXRD peaks at about 22.9 °2θ, about 4.9 °2θ, about 10.9 °2θ, about 13.3 °2θ, about 13.6 °2θ, about 15.1 °2θ, about 17.5 °2θ, about 18. 4 °2θ, about 19.0 °2θ, about 19.5 °2θ, about 20.0 °2θ, about 20.9 °2θ, and about 26.1 °2θ (±0.2 °2θ; ±0.1 °2θ; or ±0.0 °2θ; Cu Kα1 radiation). In some embodiments, Form B can be characterized by PXRD peaks at about 22.9 °2θ, about 4.9 °2θ, about 10.9 °2θ, about 13.3 °2θ, about 13.6 °2θ, about 15.1 °2θ, about 17.5 °2θ, about 18. 4 °2θ, about 19.0 °2θ, about 19.5 °2θ, about 20.0 °2θ, about 20.3 °2θ, about 20.9 °2θ, and about 26.1 °2θ (±0.2 °2θ; ±0.1 °2θ; or ±0.0 °2θ; Cu Kα1 radiation). In some embodiments, Form B can be characterized by PXRD peaks at about 22.9 °2θ, about 4.9 °2θ, about 10.9 °2θ, about 13.3 °2θ, about 13.6 °2θ, about 15.1 °2θ, about 17.5 °2θ, about 18. 4 °2θ, about 19.0 °2θ, about 19.5 °2θ, about 20.0 °2θ, about 20.3 °2θ, about 20.4 °2θ, about 20.9 °2θ, and about 26.1 °2θ (±0.2 °2θ; ±0.1 °2θ; or ±0.0 °2θ; Cu Kα1 radiation). In some embodiments, Form B can be characterized by PXRD peaks at about 22.9 °2θ, about 4.9 °2θ, about 10.9 °2θ, about 13.3 °2θ, about 13.6 °2θ, about 15.1 °2θ, about 17.5 °2θ, about 18. 4 °2θ, about 19.0 °2θ, about 19.5 °2θ, about 20.0 °2θ, about 20.3 °2θ, about 20.4 °2θ, about 20.9 °2θ, about 23.3 °2θ, and about 26.1 °2θ (±0.2 °2θ; ±0.1 °2θ; or ±0.0 °2θ; Cu Kα1 radiation). In some embodiments, Form B can be characterized by PXRD peaks at about 22.9 °2θ, about 4.9 °2θ, about 10.9 °2θ, about 13.3 °2θ, about 13.6 °2θ, about 15.1 °2θ, about 17.5 °2θ, about 18. 4 °2θ, about 19.0 °2θ, about 19.5 °2θ, about 20.0 °2θ, about 20.3 °2θ, about 20.4 °2θ, about 20.9 °2θ, about 23.3 °2θ, about 25.7 °2θ, and about 26.1 °2θ (±0.2 °2θ; ±0.1 °2θ; or ±0.0 °2θ; Cu Kα1 radiation).

In some embodiments, morphic Form B is characterized by one peak selected from about 4.9 °2θ, 10.9 °2θ, 13.3 °2θ, 13.6 °2θ, 15.1 °2θ, 17.5 °2θ, 18.4 °2θ, 19.0 °2θ, 19.5 °2θ, 20.0 °2θ, 20.3 °2θ, 20.4 °2θ, 20.9 °2θ, 22.9 °2θ, 23.3 °2θ, 25.7 °2θ, and 26.1 °2θ (±0.2 °2θ; ±0.1 °2θ; or ±0.0 °2θ; Cu Kα1 radiation). In some embodiments, morphic Form B is characterized by two peaks selected from about 4.9 °2θ, 10.9 °2θ, 13.3 °2θ, 13.6 °2θ, 15.1 °2θ, 17.5 °2θ, 18.4 °2θ, 19.0 °2θ, 19.5 °2θ, 20.0 °2θ, 20.3 °2θ, 20.4 °2θ, 20.9 °2θ, 22.9 °2θ, 23.3 °2θ, 25.7 °2θ, and 26.1 °2θ (±0.2 °2θ; ±0.1 °2θ; or ±0.0 °2θ; Cu Kα1 radiation). In some embodiments, morphic Form B is characterized by three peaks selected from about 4.9 °2θ, 10.9 °2θ, 13.3 °2θ, 13.6 °2θ, 15.1 °2θ, 17.5 °2θ, 18.4 °2θ, 19.0 °2θ, 19.5 °2θ, 20.0 °2θ, 20.3 °2θ, 20.4 °2θ, 20.9 °2θ, 22.9 °2θ, 23.3 °2θ, 25.7 °2θ, and 26.1 °2θ (±0.2 °2θ; ±0.1 °2θ; or ±0.0 °2θ; Cu Kα1 radiation). In some embodiments, morphic Form B is characterized by four peaks selected from about 4.9 °2θ, 10.9 °2θ, 13.3 °2θ, 13.6 °2θ, 15.1 °2θ, 17.5 °2θ, 18.4 °2θ, 19.0 °2θ, 19.5 °2θ, 20.0 °2θ, 20.3 °2θ, 20.4 °2θ, 20.9 °2θ, 22.9 °2θ, 23.3 °2θ, 25.7 °2θ, and 26.1 °2θ (±0.2 °2θ; ±0.1 °2θ; or ±0.0 °2θ; Cu Kα1 radiation). In some embodiments, morphic Form B is characterized by five peaks selected from about 4.9 °2θ, 10.9 °2θ, 13.3 °2θ, 13.6 °2θ, 15.1 °2θ, 17.5 °2θ, 18.4 °2θ, 19.0 °2θ, 19.5 °2θ, 20.0 °2θ, 20.3 °2θ, 20.4 °2θ, 20.9 °2θ, 22.9 °2θ, 23.3 °2θ, 25.7 °2θ, and 26.1 °2θ (±0.2 °2θ; ±0.1 °2θ; or ±0.0 °2θ; Cu Kα1 radiation). In some embodiments, morphic Form B is characterized by six peaks selected from about 4.9 °2θ, 10.9 °2θ, 13.3 °2θ, 13.6 °2θ, 15.1 °2θ, 17.5 °2θ, 18.4 °2θ, 19.0 °2θ, 19.5 °2θ, 20.0 °2θ, 20.3 °2θ, 20.4 °2θ, 20.9 °2θ, 22.9 °2θ, 23.3 °2θ, 25.7 °2θ, and 26.1 °2θ (±0.2 °2θ; ±0.1 °2θ; or ±0.0 °2θ; Cu Kα1 radiation). In some embodiments, morphic Form B is characterized by seven peaks selected from about 4.9 °2θ, 10.9 °2θ, 13.3 °2θ, 13.6 °2θ, 15.1 °2θ, 17.5 °2θ, 18.4 °2θ, 19.0 °2θ, 19.5 °2θ, 20.0 °2θ, 20.3 °2θ, 20.4 °2θ, 20.9 °2θ, 22.9 °2θ, 23.3 °2θ, 25.7 °2θ, and 26.1 °2θ (±0.2 °2θ; ±0.1 °2θ; or ±0.0 °2θ; Cu Kα1 radiation). In some embodiments, morphic Form B is characterized by eight peaks selected from about 4.9 °2θ, 10.9 °2θ, 13.3 °2θ, 13.6 °2θ, 15.1 °2θ, 17.5 °2θ, 18.4 °2θ, 19.0 °2θ, 19.5 °2θ, 20.0 °2θ, 20.3 °2θ, 20.4 °2θ, 20.9 °2θ, 22.9 °2θ, 23.3 °2θ, 25.7 °2θ, and 26.1 °2θ (±0.2 °2θ; ±0.1 °2θ; or ±0.0 °2θ; Cu Kα1 radiation). In some embodiments, morphic Form B is characterized by nine peaks selected from about 4.9 °2θ, 10.9 °2θ, 13.3 °2θ, 13.6 °2θ, 15.1 °2θ, 17.5 °2θ, 18.4 °2θ, 19.0 °2θ, 19.5 °2θ, 20.0 °2θ, 20.3 °2θ, 20.4 °2θ, 20.9 °2θ, 22.9 °2θ, 23.3 °2θ, 25.7 °2θ, and 26.1 °2θ (±0.2 °2θ; ±0.1 °2θ; or ±0.0 °2θ; Cu Kα1 radiation). In some embodiments, morphic Form B is characterized by ten peaks selected from about 4.9 °2θ, 10.9 °2θ, 13.3 °2θ, 13.6 °2θ, 15.1 °2θ, 17.5 °2θ, 18.4 °2θ, 19.0 °2θ, 19.5 °2θ, 20.0 °2θ, 20.3 °2θ, 20.4 °2θ, 20.9 °2θ, 22.9 °2θ, 23.3 °2θ, 25.7 °2θ, and 26.1 °2θ (±0.2 °2θ; ±0.1 °2θ; or ±0.0 °2θ; Cu Kα1 radiation). In some embodiments, morphic Form B is characterized by eleven peaks selected from about 4.9 °2θ, 10.9 °2θ, 13.3 °2θ, 13.6 °2θ, 15.1 °2θ, 17.5 °2θ, 18.4 °2θ, 19.0 °2θ, 19.5 °2θ, 20.0 °2θ, 20.3 °2θ, 20.4 °2θ, 20.9 °2θ, 22.9 °2θ, 23.3 °2θ, 25.7 °2θ, and 26.1 °2θ (±0.2 °2θ; ±0.1 °2θ; or ±0.0 °2θ; Cu Kα1 radiation). In some embodiments, morphic Form B is characterized by twelve peaks selected from about 4.9 °2θ, 10.9 °2θ, 13.3 °2θ, 13.6 °2θ, 15.1 °2θ, 17.5 °2θ, 18.4 °2θ, 19.0 °2θ, 19.5 °2θ, 20.0 °2θ, 20.3 °2θ, 20.4 °2θ, 20.9 °2θ, 22.9 °2θ, 23.3 °2θ, 25.7 °2θ, and 26.1 °2θ (±0.2 °2θ; ±0.1 °2θ; or ±0.0 °2θ; Cu Kα1 radiation). In some embodiments, morphic Form B is characterized by thirteen peaks selected from about 4.9 °2θ, 10.9 °2θ, 13.3 °2θ, 13.6 °2θ, 15.1 °2θ, 17.5 °2θ, 18.4 °2θ, 19.0 °2θ, 19.5 °2θ, 20.0 °2θ, 20.3 °2θ, 20.4 °2θ, 20.9 °2θ, 22.9 °2θ, 23.3 °2θ, 25.7 °2θ, and 26.1 °2θ (±0.2 °2θ; ±0.1 °2θ; or ±0.0 °2θ; Cu Kα1 radiation). In some embodiments, morphic Form B is characterized by fourteen peaks selected from about 4.9 °2θ, 10.9 °2θ, 13.3 °2θ, 13.6 °2θ, 15.1 °2θ, 17.5 °2θ, 18.4 °2θ, 19.0 °2θ, 19.5 °2θ, 20.0 °2θ, 20.3 °2θ, 20.4 °2θ, 20.9 °2θ, 22.9 °2θ, 23.3 °2θ, 25.7 °2θ, and 26.1 °2θ (±0.2 °2θ; ±0.1 °2θ; or ±0.0 °2θ; Cu Kα1 radiation). In some embodiments, morphic Form B is characterized by fifteen peaks selected from about 4.9 °2θ, 10.9 °2θ, 13.3 °2θ, 13.6 °2θ, 15.1 °2θ, 17.5 °2θ, 18.4 °2θ, 19.0 °2θ, 19.5 °2θ, 20.0 °2θ, 20.3 °2θ, 20.4 °2θ, 20.9 °2θ, 22.9 °2θ, 23.3 °2θ, 25.7 °2θ, and 26.1 °2θ (±0.2 °2θ; ±0.1 °2θ; or ±0.0 °2θ; Cu Kα1 radiation). In some embodiments, morphic Form B is characterized by sixteen peaks selected from about 4.9 °2θ, 10.9 °2θ, 13.3 °2θ, 13.6 °2θ, 15.1 °2θ, 17.5 °2θ, 18.4 °2θ, 19.0 °2θ, 19.5 °2θ, 20.0 °2θ, 20.3 °2θ, 20.4 °2θ, 20.9 °2θ, 22.9 °2θ, 23.3 °2θ, 25.7 °2θ, and 26.1 °2θ (±0.2 °2θ; ±0.1 °2θ; or ±0.0 °2θ; Cu Kα1 radiation). In some embodiments, morphic Form B is characterized by seventeen peaks selected from about 4.9 °2θ, 10.9 °2θ, 13.3 °2θ, 13.6 °2θ, 15.1 °2θ, 17.5 °2θ, 18.4 °2θ, 19.0 °2θ, 19.5 °2θ, 20.0 °2θ, 20.3 °2θ, 20.4 °2θ, 20.9 °2θ, 22.9 °2θ, 23.3 °2θ, 25.7 °2θ, and 26.1 °2θ (±0.2 °2θ; ±0.1 °2θ; or ±0.0 °2θ; Cu Kα1 radiation).

Accordingly, in some embodiments, morphic Form B is characterized by one, two, three, four, five, six, seven, eight, nine, ten, eleven, twelve, thirteen, fourteen, fifteen, sixteen or seventeen peaks selected from about 4.9, 10.9, 13.3, 13.6, 15.1, 17.5, 18.4, 19.0, 19.5, 20.0, 20.3, 20.4, 20.9, 22.9, 23.3, 25.7, and 26.1 °2θ (Cu Kα1 radiation).

### Form C

In some embodiments, the disclosure is directed methods of treating and/or preventing a SARS-CoV-2 infection and/or a SARS-CoV-2 infection-associated disease or disorder (e.g. COVID-19) with Compound 1 Form C.

Morphic Form C can be characterized by a PXRD peak at about 22.4 °2θ (±0.2 °2θ; ±0.1 °2θ; or ±0.0 °2θ; Cu Kα1 radiation). Morphic Form C can further be characterized by a PXRD peak at about 4.9 °2θ, 10.7 °2θ, 13.2 °2θ, 17.4 °2θ, 19.4 °2θ, 20.5 °2θ, 22.8 °2θ, 25.2 °2θ, and/or 25.6, °2θ (±0.2 °2θ; ±0.1 °2θ; or ±0.0 °2θ; Cu Kα1 radiation). Morphic Form C can further be characterized by a PXRD peak at about 6.6 °2θ, 11.2 °2θ, 13.5 °2θ, 14.2 °2θ, 14.8 °2θ, 16.5 °2θ, 16.7 °2θ, 18.5 °2θ, 19.0 °2θ, 19.7 °2θ, 20.3 °2θ, 21.0 °2θ, 25.4 °2θ, and/or 26.7 °2θ (±0.2 °2θ; ±0.1 °2θ; or ±0.0 °2θ; Cu Kα1 radiation).

In some embodiments, morphic Form C can be characterized by PXRD peaks at about 4.9 °2θ, about 10.7 °2θ, and about 13.2 °2θ (±0.2 °2θ; ±0.1 °2θ; or ±0.0 °2θ; Cu Kα1 radiation). In some embodiments, morphic Form C can be characterized by PXRD peaks at about 10.7 °2θ, about 13.2 °2θ, and about 17.4 °2θ (±0.2 °2θ; ±0.1 °2θ; or ±0.0 °2θ; Cu Kα1 radiation). In some embodiments, morphic Form C can be characterized by PXRD peaks at about 13.2 °2θ, about 17.4 °2θ, and about 19.4 °2θ (±0.2 °2θ; ±0.1 °2θ; or ±0.0 °2θ; Cu Kα1 radiation). In some embodiments, morphic Form C can be characterized by PXRD peaks at about 17.4 °2θ, about 19.4 °2θ, and about 20.5 °2θ (±0.2 °2θ; ±0.1 °2θ; or ±0.0 °2θ; Cu Kα1 radiation). In some embodiments, morphic Form C can be characterized by PXRD peaks at about 19.4 °2θ, about 20.5 °2θ, and about 22.4 °2θ (±0.2 °2θ; ±0.1 °2θ; or ±0.0 °2θ; Cu Kα1 radiation). In some embodiments, morphic Form C can be characterized by PXRD peaks at about 20.5 °2θ, about 22.4 °2θ, and about 22.8 °2θ (±0.2 °2θ; ±0.1 °2θ; or ±0.0 °2θ; Cu Kα1 radiation). In some embodiments, morphic Form C can be characterized by PXRD peaks at about 22.4 °2θ, about 22.8 °2θ, and about 25.2 °2θ (±0.2 °2θ; ±0.1 °2θ; or ±0.0 °2θ; Cu Kα1 radiation). In some embodiments, morphic Form C can be characterized by PXRD peaks at about 22.8 °2θ, about 25.2 °2θ, and about 25.6 °2θ (±0.2 °2θ; ±0.1 °2θ; or ±0.0 °2θ; Cu Kα1 radiation).

In some embodiments, morphic Form C can be characterized by a PXRD peak at about 22.4 °2θ and about 4.9 °2θ (±0.2 °2θ; ±0.1 °2θ; or ±0.0 °2θ; Cu Kα1 radiation). In some embodiments, morphic Form C can be characterized by a PXRD peak at about 22.4 °2θ about 4.9 °2θ, and about 10.7 °2θ (±0.2 °2θ; ±0.1 °2θ; or ±0.0 °2θ; Cu Kα1 radiation). In some embodiments, morphic Form C can be characterized by a PXRD peak at about 22.4 °2θ about 4.9 °2θ, about 10.7 °2θ, and about 13.2 °2θ (±0.2 °2θ; ±0.1 °2θ; or ±0.0 °2θ; Cu Kα1 radiation). In some embodiments, morphic Form C can be characterized by a PXRD peak at about 22.4 °2θ about 4.9 °2θ, about 10.7 °2θ, about 13.2 °2θ, and about 17.4 °2θ (±0.2 °2θ; ±0.1 °2θ; or ±0.0 °2θ; Cu Kα1 radiation). In some embodiments, morphic Form C can be characterized by a PXRD peak at about 22.4 °2θ about 4.9 °2θ, about 10.7 °2θ, about 13.2 °2θ, about 17.4 °2θ, and about 19.4 °2θ (±0.2 °2θ; ±0.1 °2θ; or ±0.0 °2θ; Cu Kα1 radiation). In some embodiments, morphic Form C can be characterized by a PXRD peak at about 22.4 °2θ about 4.9 °2θ, about 10.7 °2θ, about 13.2 °2θ, about 17.4 °2θ, about 19.4 °2θ, and about 20.5 °2θ (±0.2 °2θ; ±0.1 °2θ; or ±0.0 °2θ; Cu Kα1 radiation). In some embodiments, morphic Form C can be characterized by a PXRD peak at about 22.4 °2θ about 4.9 °2θ, about 10.7 °2θ, about 13.2 °2θ, about 17.4 °2θ, about 19.4 °2θ, about 20.5 °2θ, and about 22.8 °2θ (±0.2 °2θ; ±0.1 °2θ; or ±0.0 °2θ; Cu Kα1 radiation). In some embodiments, morphic Form C can be characterized by a PXRD peak at about 22.4 °2θ about 4.9 °2θ, about 10.7 °2θ, about 13.2 °2θ, about 17.4 °2θ, about 19.4 °2θ, about 20.5 °2θ, about 22.8 °2θ, and about 25.2 °2θ (±0.2 °2θ; ±0.1 °2θ; or ±0.0 °2θ; Cu Kα1 radiation). In some embodiments, morphic Form C can be characterized by a PXRD peak at about 22.4 °2θ about 4.9 °2θ, about 10.7 °2θ, about 13.2 °2θ, about 17.4 °2θ, about 19.4 °2θ, about 20.5 °2θ, about 22.8 °2θ, about 25.2 °2θ, and about 25.6 °2θ (±0.2 °2θ; ±0.1 °2θ; or ±0.0 °2θ; Cu Kα1 radiation).

In some embodiments, morphic Form C can be characterized by a PXRD peak at about 22.4 °2θ about 4.9 °2θ, about 6.6 °2θ, about 10.7 °2θ, about 13.2 °2θ, about 17.4 °2θ, about 19.4 °2θ, about 20.5 °2θ, about 22.8 °2θ, about 25.2 °2θ, and about 25.6 °2θ (±0.2 °2θ; ±0.1 °2θ; or ±0.0 °2θ; Cu Kα1 radiation). In some embodiments, morphic Form C can be characterized by a PXRD peak at about 22.4 °2θ about 4.9 °2θ, about 6.6 °2θ, about 10.7 °2θ, about 11.2 °2θ, about 13.2 °2θ, about 17.4 °2θ, about 19.4 °2θ, about 20.5 °2θ, about 22.8 °2θ, about 25.2 °2θ, and about 25.6 °2θ (±0.2 °2θ; ±0.1 °2θ; or ±0.0 °2θ; Cu Kα1 radiation). In some embodiments, morphic Form C can be characterized by a PXRD peak at about 22.4 °2θ about 4.9 °2θ, about 6.6 °2θ, about 10.7 °2θ, about 11.2 °2θ, about 13.2 °2θ, about 13.5 °2θ, about 17.4 °2θ, about 19.4 °2θ, about 20.5 °2θ, about 22.8 °2θ, about 25.2 °2θ, and about 25.6 °2θ (±0.2 °2θ; ±0.1 °2θ; or ±0.0 °2θ; Cu Kα1 radiation). In some embodiments, morphic Form C can be characterized by a PXRD peak at about 22.4 °2θ about 4.9 °2θ, about 6.6 °2θ, about 10.7 °2θ, about 11.2 °2θ, about 13.2 °2θ, about 13.5 °2θ, about 14.2 °2θ, about 17.4 °2θ, about 19.4 °2θ, about 20.5 °2θ, about 22.8 °2θ, about 25.2 °2θ, and about 25.6 °2θ (±0.2 °2θ; ±0.1 °2θ; or ±0.0 °2θ; Cu Kα1 radiation). In some embodiments, morphic Form C can be characterized by a PXRD peak at about 22.4 °2θ about 4.9 °2θ, about 6.6 °2θ, about 10.7 °2θ, about 11.2 °2θ, about 13.2 °2θ, about 13.5 °2θ, about 14.2 °2θ, about 14.8 °2θ, about 17.4 °2θ, about 19.4 °2θ, about 20.5 °2θ, about 22.8 °2θ, about 25.2 °2θ, and about 25.6 °2θ (±0.2 °2θ; ±0.1 °2θ; or ±0.0 °2θ; Cu Kα1 radiation). In some embodiments, morphic Form C can be characterized by a PXRD peak at about 22.4 °2θ about 4.9 °2θ, about 6.6 °2θ, about 10.7 °2θ, about 11.2 °2θ, about 13.2 °2θ, about 13.5 °2θ, about 14.2 °2θ, about 14.8 °2θ, about 16.5 °2θ, about 17.4 °2θ, about 19.4 °2θ, about 20.5 °2θ, about 22.8 °2θ, about 25.2 °2θ, and about 25.6 °2θ (±0.2 °2θ; ±0.1 °2θ; or ±0.0 °2θ; Cu Kα1 radiation). In some embodiments, morphic Form C can be characterized by a PXRD peak at about 22.4 °2θ about 4.9 °2θ, about 6.6 °2θ, about 10.7 °2θ, about 11.2 °2θ, about 13.2 °2θ, about 13.5 °2θ, about 14.2 °2θ, about 14.8 °2θ, about 16.5 °2θ, about 16.7 °2θ, about 17.4 °2θ, about 19.4 °2θ, about 20.5 °2θ, about 22.8 °2θ, about 25.2 °2θ, and about 25.6 °2θ (±0.2 °2θ; ±0.1 °2θ; or ±0.0 °2θ; Cu Kα1 radiation). In some embodiments, morphic Form C can be characterized by a PXRD peak at about 22.4 °2θ about 4.9 °2θ, about 6.6 °2θ, about 10.7 °2θ, about 11.2 °2θ, about 13.2 °2θ, about 13.5 °2θ, about 14.2 °2θ, about 14.8 °2θ, about 16.5 °2θ, about 16.7 °2θ, about 17.4 °2θ, about 18.5 °2θ, about 19.4 °2θ, about 20.5 °2θ, about 22.8 °2θ, about 25.2 °2θ, and about 25.6 °2θ (±0.2 °2θ; ±0.1 °2θ; or ±0.0 °2θ; Cu Kα1 radiation). In some embodiments, morphic Form C can be characterized by a PXRD peak at about 22.4 °2θ about 4.9 °2θ, about 6.6 °2θ, about 10.7 °2θ, about 11.2 °2θ, about 13.2 °2θ, about 13.5 °2θ, about 14.2 °2θ, about 14.8 °2θ, about 16.5 °2θ, about 16.7 °2θ, about 17.4 °2θ, about 18.5 °2θ, about 19.0 °2θ, about 19.4 °2θ, about 20.5 °2θ, about 22.8 °2θ, about 25.2 °2θ, and about 25.6 °2θ (±0.2 °2θ; ±0.1 °2θ; or ±0.0 °2θ; Cu Kα1 radiation). In some embodiments, morphic Form C can be characterized by a PXRD peak at about 22.4 °2θ about 4.9 °2θ, about 6.6 °2θ, about 10.7 °2θ, about 11.2 °2θ, about 13.2 °2θ, about 13.5 °2θ, about 14.2 °2θ, about 14.8 °2θ, about 16.5 °2θ, about 16.7 °2θ, about 17.4 °2θ, about 18.5 °2θ, about 19.0 °2θ, about 19.4 °2θ, about 19.7 °2θ, about 20.5 °2θ, about 22.8 °2θ, about 25.2 °2θ, and about 25.6 °2θ (±0.2 °2θ; ±0.1 °2θ; or ±0.0 °2θ; Cu Kα1 radiation). In some embodiments, morphic Form C can be characterized by a PXRD peak at about 22.4 °2θ about 4.9 °2θ, about 6.6 °2θ, about 10.7 °2θ, about 11.2 °2θ, about 13.2 °2θ, about 13.5 °2θ, about 14.2 °2θ, about 14.8 °2θ, about 16.5 °2θ, about 16.7 °2θ, about 17.4 °2θ, about 18.5 °2θ, about 19.0 °2θ, about 19.4 °2θ, about 19.7 °2θ, about 20.3 °2θ, about 20.5 °2θ, about 22.8 °2θ, about 25.2 °2θ, and about 25.6 °2θ (±0.2 °2θ; ±0.1 °2θ; or ±0.0 °2θ; Cu Kα1 radiation). In some embodiments, morphic Form C can be characterized by a PXRD peak at about 22.4 °2θ about 4.9 °2θ, about 6.6 °2θ, about 10.7 °2θ, about 11.2 °2θ, about 13.2 °2θ, about 13.5 °2θ, about 14.2 °2θ, about 14.8 °2θ, about 16.5 °2θ, about 16.7 °2θ, about 17.4 °2θ, about 18.5 °2θ, about 19.0 °2θ, about 19.4 °2θ, about 19.7 °2θ, about 20.3 °2θ, about 20.5 °2θ, about 21.0 °2θ, about 22.8 °2θ, about 25.2 °2θ, and about 25.6 °2θ (±0.2 °2θ; ±0.1 °2θ; or ±0.0 °2θ; Cu Kα1 radiation). In some embodiments, morphic Form C can be characterized by a PXRD peak at about 22.4 °2θ about 4.9 °2θ, about 6.6 °2θ, about 10.7 °2θ, about 11.2 °2θ, about 13.2 °2θ, about 13.5 °2θ, about 14.2 °2θ, about 14.8 °2θ, about 16.5 °2θ, about 16.7 °2θ, about 17.4 °2θ, about 18.5 °2θ, about 19.0 °2θ, about 19.4 °2θ, about 19.7 °2θ, about 20.3 °2θ, about 20.5 °2θ, about 21.0 °2θ, about 22.8 °2θ, about 25.2 °2θ, about 25.4 °2θ, and about 25.6 °2θ (±0.2 °2θ; ±0.1 °2θ; or ±0.0 °2θ; Cu Kα1 radiation). In some embodiments, morphic Form C can be characterized by a PXRD peak at about 22.4 °2θ about 4.9 °2θ, about 6.6 °2θ, about 10.7 °2θ, about 11.2 °2θ, about 13.2 °2θ, about 13.5 °2θ, about 14.2 °2θ, about 14.8 °2θ, about 16.5 °2θ, about 16.7 °2θ, about 17.4 °2θ, about 18.5 °2θ, about 19.0 °2θ, about 19.4 °2θ, about 19.7 °2θ, about 20.3 °2θ, about 20.5 °2θ, about 21.0 °2θ, about 22.8 °2θ, about 25.2 °2θ, about 25.4 °2θ, about 25.6 °2θ, and about 26.7 °2θ (±0.2 °2θ; ±0.1 °2θ; or ±0.0 °2θ; Cu Kα1 radiation).

Accordingly, in some embodiments, Form C can be characterized by one, two, three, four, five, six, seven, eight, nine, ten, eleven, twelve, thirteen, fourteen, fifteen, sixteen, seventeen, eighteen, nineteen, twenty, twenty-one, twenty-two, twenty-three or twenty-four PXRD peaks selected from about 4.9, 6.6, 10.7, 11.2, 13.2, 13.5, 14.2, 14.8, 16.5, 16.7, 17.4, 18.5, 19.0, 19.4, 19.7, 20.3, 20.5, 21.0, 22.8, 22.4, 25.2, 25.4, 25.6, and 26.7 °2θ (Cu Kα1 radiation). In some embodiments, Form C can be characterized by one PXRD peak selected from about 4.9, 6.6, 10.7, 11.2, 13.2, 13.5, 14.2, 14.8, 16.5, 16.7, 17.4, 18.5, 19.0, 19.4, 19.7, 20.3, 20.5, 21.0, 22.8, 22.4, 25.2, 25.4, 25.6, and 26.7 °2θ (±0.2 °2θ; ±0.1 °2θ; or ±0.0 °2θ; Cu Kα1 radiation). In some embodiments, Form C can be characterized by two PXRD peaks selected from about 4.9, 6.6, 10.7, 11.2, 13.2, 13.5, 14.2, 14.8, 16.5, 16.7, 17.4, 18.5, 19.0, 19.4, 19.7, 20.3, 20.5, 21.0, 22.8, 22.4, 25.2, 25.4, 25.6, and 26.7 °2θ (±0.2 °2θ; ±0.1 °2θ; or ±0.0 °2θ; Cu Kα1 radiation). In some embodiments, Form C can be characterized by three PXRD peaks selected from about 4.9, 6.6, 10.7, 11.2, 13.2, 13.5, 14.2, 14.8, 16.5, 16.7, 17.4, 18.5, 19.0, 19.4, 19.7, 20.3, 20.5, 21.0, 22.8, 22.4, 25.2, 25.4, 25.6, and 26.7 °2θ (±0.2 °2θ; ±0.1 °2θ; or ±0.0 °2θ; Cu Kα1 radiation). In some embodiments, Form C can be characterized by four PXRD peaks selected from about 4.9, 6.6, 10.7, 11.2, 13.2, 13.5, 14.2, 14.8, 16.5, 16.7, 17.4, 18.5, 19.0, 19.4, 19.7, 20.3, 20.5, 21.0, 22.8, 22.4, 25.2, 25.4, 25.6, and 26.7 °2θ (±0.2 °2θ; ±0.1 °2θ; or ±0.0 °2θ; Cu Kα1 radiation). In some embodiments, Form C can be characterized by five PXRD peaks selected from about 4.9, 6.6, 10.7, 11.2, 13.2, 13.5, 14.2, 14.8, 16.5, 16.7, 17.4, 18.5, 19.0, 19.4, 19.7, 20.3, 20.5, 21.0, 22.8, 22.4, 25.2, 25.4, 25.6, and 26.7 °2θ (±0.2 °2θ; ±0.1 °2θ; or ±0.0 °2θ; Cu Kα1 radiation). In some embodiments, Form C can be characterized by six PXRD peaks selected from about 4.9, 6.6, 10.7, 11.2, 13.2, 13.5, 14.2, 14.8, 16.5, 16.7, 17.4, 18.5, 19.0, 19.4, 19.7, 20.3, 20.5, 21.0, 22.8, 22.4, 25.2, 25.4, 25.6, and 26.7 °2θ (±0.2 °2θ; ±0.1 °2θ; or ±0.0 °2θ; Cu Kα1 radiation). In some embodiments, Form C can be characterized by seven PXRD peaks selected from about 4.9, 6.6, 10.7, 11.2, 13.2, 13.5, 14.2, 14.8, 16.5, 16.7, 17.4, 18.5, 19.0, 19.4, 19.7, 20.3, 20.5, 21.0, 22.8, 22.4, 25.2, 25.4, 25.6, and 26.7 °2θ (±0.2 °2θ; ±0.1 °2θ; or ±0.0 °2θ; Cu Kα1 radiation). In some embodiments, Form C can be characterized by eight PXRD peaks selected from about 4.9, 6.6, 10.7, 11.2, 13.2, 13.5, 14.2, 14.8, 16.5, 16.7, 17.4, 18.5, 19.0, 19.4, 19.7, 20.3, 20.5, 21.0, 22.8, 22.4, 25.2, 25.4, 25.6, and 26.7 °2θ (±0.2 °2θ; ±0.1 °2θ; or ±0.0 °2θ; Cu Kα1 radiation). In some embodiments, Form C can be characterized by nine PXRD peaks selected from about 4.9, 6.6, 10.7, 11.2, 13.2, 13.5, 14.2, 14.8, 16.5, 16.7, 17.4, 18.5, 19.0, 19.4, 19.7, 20.3, 20.5, 21.0, 22.8, 22.4, 25.2, 25.4, 25.6, and 26.7 °2θ (±0.2 °2θ; ±0.1 °2θ; or ±0.0 °2θ; Cu Kα1 radiation). In some embodiments, Form C can be characterized by ten PXRD peaks selected from about 4.9, 6.6, 10.7, 11.2, 13.2, 13.5, 14.2, 14.8, 16.5, 16.7, 17.4, 18.5, 19.0, 19.4, 19.7, 20.3, 20.5, 21.0, 22.8, 22.4, 25.2, 25.4, 25.6, and 26.7 °2θ (±0.2 °2θ; ±0.1 °2θ; or ±0.0 °2θ; Cu Kα1 radiation). In some embodiments, Form C can be characterized by eleven PXRD peaks selected from about 4.9, 6.6, 10.7, 11.2, 13.2, 13.5, 14.2, 14.8, 16.5, 16.7, 17.4, 18.5, 19.0, 19.4, 19.7, 20.3, 20.5, 21.0, 22.8, 22.4, 25.2, 25.4, 25.6, and 26.7 °2θ (±0.2 °2θ; ±0.1 °2θ; or ±0.0 °2θ; Cu Kα1 radiation). In some embodiments, Form C can be characterized by twelve PXRD peaks selected from about 4.9, 6.6, 10.7, 11.2, 13.2, 13.5, 14.2, 14.8, 16.5, 16.7, 17.4, 18.5, 19.0, 19.4, 19.7, 20.3, 20.5, 21.0, 22.8, 22.4, 25.2, 25.4, 25.6, and 26.7 °2θ (±0.2 °2θ; ±0.1 °2θ; or ±0.0 °2θ; Cu Kα1 radiation). In some embodiments, Form C can be characterized by thirteen PXRD peaks selected from about 4.9, 6.6, 10.7, 11.2, 13.2, 13.5, 14.2, 14.8, 16.5, 16.7, 17.4, 18.5, 19.0, 19.4, 19.7, 20.3, 20.5, 21.0, 22.8, 22.4, 25.2, 25.4, 25.6, and 26.7 °2θ (±0.2 °2θ; ±0.1 °2θ; or ±0.0 °2θ; Cu Kα1 radiation). In some embodiments, Form C can be characterized by fourteen PXRD peaks selected from about 4.9, 6.6, 10.7, 11.2, 13.2, 13.5, 14.2, 14.8, 16.5, 16.7, 17.4, 18.5, 19.0, 19.4, 19.7, 20.3, 20.5, 21.0, 22.8, 22.4, 25.2, 25.4, 25.6, and 26.7 °2θ (±0.2 °2θ; ±0.1 °2θ; or ±0.0 °2θ; Cu Kα1 radiation). In some embodiments, Form C can be characterized by fifteen PXRD peaks selected from about 4.9, 6.6, 10.7, 11.2, 13.2, 13.5, 14.2, 14.8, 16.5, 16.7, 17.4, 18.5, 19.0, 19.4, 19.7, 20.3, 20.5, 21.0, 22.8, 22.4, 25.2, 25.4, 25.6, and 26.7 °2θ (±0.2 °2θ; ±0.1 °2θ; or ±0.0 °2θ; Cu Kα1 radiation). In some embodiments, Form C can be characterized by sixteen PXRD peaks selected from about 4.9, 6.6, 10.7, 11.2, 13.2, 13.5, 14.2, 14.8, 16.5, 16.7, 17.4, 18.5, 19.0, 19.4, 19.7, 20.3, 20.5, 21.0, 22.8, 22.4, 25.2, 25.4, 25.6, and 26.7 °2θ (±0.2 °2θ; ±0.1 °2θ; or ±0.0 °2θ; Cu Kα1 radiation). In some embodiments, Form C can be characterized by seventeen PXRD peaks selected from about 4.9, 6.6, 10.7, 11.2, 13.2, 13.5, 14.2, 14.8, 16.5, 16.7, 17.4, 18.5, 19.0, 19.4, 19.7, 20.3, 20.5, 21.0, 22.8, 22.4, 25.2, 25.4, 25.6, and 26.7 °2θ (±0.2 °2θ; ±0.1 °2θ; or ±0.0 °2θ; Cu Kα1 radiation). In some embodiments, Form C can be characterized by eighteen PXRD peaks selected from about 4.9, 6.6, 10.7, 11.2, 13.2, 13.5, 14.2, 14.8, 16.5, 16.7, 17.4, 18.5, 19.0, 19.4, 19.7, 20.3, 20.5, 21.0, 22.8, 22.4, 25.2, 25.4, 25.6, and 26.7 °2θ (±0.2 °2θ; ±0.1 °2θ; or ±0.0 °2θ; Cu Kα1 radiation). In some embodiments, Form C can be characterized by nineteen PXRD peaks selected from about 4.9, 6.6, 10.7, 11.2, 13.2, 13.5, 14.2, 14.8, 16.5, 16.7, 17.4, 18.5, 19.0, 19.4, 19.7, 20.3, 20.5, 21.0, 22.8, 22.4, 25.2, 25.4, 25.6, and 26.7 °2θ (±0.2 °2θ; ±0.1 °2θ; or ±0.0 °2θ; Cu Kα1 radiation). In some embodiments, Form C can be characterized by twenty PXRD peaks selected from about 4.9, 6.6, 10.7, 11.2, 13.2, 13.5, 14.2, 14.8, 16.5, 16.7, 17.4, 18.5, 19.0, 19.4, 19.7, 20.3, 20.5, 21.0, 22.8, 22.4, 25.2, 25.4, 25.6, and 26.7 °2θ (±0.2 °2θ; ±0.1 °2θ; or ±0.0 °2θ; Cu Kα1 radiation). In some embodiments, Form C can be characterized by twenty-one PXRD peaks selected from about 4.9, 6.6, 10.7, 11.2, 13.2, 13.5, 14.2, 14.8, 16.5, 16.7, 17.4, 18.5, 19.0, 19.4, 19.7, 20.3, 20.5, 21.0, 22.8, 22.4, 25.2, 25.4, 25.6, and 26.7 °2θ (±0.2 °2θ; ±0.1 °2θ; or ±0.0 °2θ; Cu Kα1 radiation). In some embodiments, Form C can be characterized by twenty-two PXRD peaks selected from about 4.9, 6.6, 10.7, 11.2, 13.2, 13.5, 14.2, 14.8, 16.5, 16.7, 17.4, 18.5, 19.0, 19.4, 19.7, 20.3, 20.5, 21.0, 22.8, 22.4, 25.2, 25.4, 25.6, and 26.7 °2θ (±0.2 °2θ; ±0.1 °2θ; or ±0.0 °2θ; Cu Kα1 radiation). In some embodiments, Form C can be characterized by twenty-three PXRD peaks selected from about 4.9, 6.6, 10.7, 11.2, 13.2, 13.5, 14.2, 14.8, 16.5, 16.7, 17.4, 18.5, 19.0, 19.4, 19.7, 20.3, 20.5, 21.0, 22.8, 22.4, 25.2, 25.4, 25.6, and 26.7 °2θ (±0.2 °2θ; ±0.1 °2θ; or ±0.0 °2θ; Cu Kα1 radiation). In some embodiments, Form C can be characterized by twenty-four PXRD peaks selected from about 4.9, 6.6, 10.7, 11.2, 13.2, 13.5, 14.2, 14.8, 16.5, 16.7, 17.4, 18.5, 19.0, 19.4, 19.7, 20.3, 20.5, 21.0, 22.8, 22.4, 25.2, 25.4, 25.6, and 26.7 °2θ (±0.2 °2θ; ±0.1 °2θ; or ±0.0 °2θ; Cu Kα1 radiation).

### Form D

In some embodiments, the disclosure is directed methods of treating and/or preventing a SARS-CoV-2 infection and/or a SARS-CoV-2 infection-associated disease or disorder (e.g. COVID-19) with Compound 1 Form D.

Morphic Form D can be characterized by a PXRD peak at about 26.6 °2θ (±0.2 °2θ; ±0.1 °2θ; or ±0.0 °2θ; Cu Kα1 radiation). Form D can further be characterized by PXRD peaks at about 10.7 °2θ, 11.5 °2θ, 12.3 °2θ, 15.4 °2θ, 18.8 °2θ, 23.6 °2θ, and/or 26.8 °2θ (±0.2 °2θ; ±0.1 °2θ; or ±0.0 °2θ; Cu Kα1 radiation). Form D can further be characterized by peaks at about 11.7 °2θ, 13.8 °2θ, and/or 16.4 °2θ (±0.2 °2θ; ±0.1 °2θ; or ±0.0 °2θ; Cu Kα1 radiation).

In some embodiments, morphic Form D can be characterized by PXRD peaks at about 10.7 °2θ, about 11.5 °2θ, and about 12.3 °2θ (±0.2 °2θ; ±0.1 °2θ; or ±0.0 °2θ; Cu Kα1 radiation). In some embodiments, morphic Form D can be characterized by PXRD peaks at about 11.5 °2θ, about 12.3 °2θ, and about 15.4 °2θ (±0.2 °2θ; ±0.1 °2θ; or ±0.0 °2θ; Cu Kα1 radiation). In some embodiments, morphic Form D can be characterized by PXRD peaks at about 12.3 °2θ, about 15.4 °2θ, and about 18.8 °2θ (±0.2 °2θ; ±0.1 °2θ; or ±0.0 °2θ; Cu Kα1 radiation). In some embodiments, morphic Form D can be characterized by PXRD peaks at about 15.4 °2θ, about 18.8 °2θ, and about 23.6 °2θ (±0.2 °2θ; ±0.1 °2θ; or ±0.0 °2θ; Cu Kα1 radiation). In some embodiments, morphic Form D can be characterized by PXRD peaks at about 18.8 °2θ, about 23.6 °2θ, and about 26.6 °2θ (±0.2 °2θ; ±0.1 °2θ; or ±0.0 °2θ; Cu Kα1 radiation). In some embodiments, morphic Form D can be characterized by PXRD peaks at about 23.6 °2θ, about 26.6 °2θ, and about 26.8 °2θ (±0.2 °2θ; ±0.1 °2θ; or ±0.0 °2θ; Cu Kα1 radiation).

In some embodiments, morphic Form D can be characterized by PXRD peaks at about 26.6 °2θ and about 10.7 °2θ (±0.2 °2θ; ±0.1 °2θ; or ±0.0 °2θ; Cu Kα1 radiation). In some embodiments, morphic Form D can be characterized by PXRD peaks at about 26.6 °2θ, about 10.7 °2θ and about 11.5 °2θ (±0.2 °2θ; ±0.1 °2θ; or ±0.0 °2θ; Cu Kα1 radiation). In some embodiments, morphic Form D can be characterized by PXRD peaks at about 26.6 °2θ, about 10.7 °2θ, about 11.5 °2θ, and about 12.3 °2θ (±0.2 °2θ; ±0.1 °2θ; or ±0.0 °2θ; Cu Kα1 radiation). In some embodiments, morphic Form D can be characterized by PXRD peaks at about 26.6 °2θ, about 10.7 °2θ, about 11.5 °2θ, about 12.3 °2θ, and about 15.4 °2θ (±0.2 °2θ; ±0.1 °2θ; or ±0.0 °2θ; Cu Kα1 radiation). In some embodiments, morphic Form D can be characterized by PXRD peaks at about 26.6 °2θ, about 10.7 °2θ, about 11.5 °2θ, about 12.3 °2θ, about 15.4 °2θ, and about 18.8 °2θ (±0.2 °2θ; ±0.1 °2θ; or ±0.0 °2θ; Cu Kα1 radiation). In some embodiments, morphic Form D can be characterized by PXRD peaks at about 26.6 °2θ, about 10.7 °2θ, about 11.5 °2θ, about 12.3 °2θ, about 15.4 °2θ, about 18.8 °2θ, and about 23.6 °2θ (±0.2 °2θ; ±0.1 °2θ; or ±0.0 °2θ; Cu Kα1 radiation). In some embodiments, morphic Form D can be characterized by PXRD peaks at about 26.6 °2θ, about 10.7 °2θ, about 11.5 °2θ, about 12.3 °2θ, about 15.4 °2θ, about 18.8 °2θ, about 23.6 °2θ, and about 26.8 °2θ (±0.2 °2θ; ±0.1 °2θ; or ±0.0 °2θ; Cu Kα1 radiation).

In some embodiments, morphic Form D can be characterized by PXRD peaks at about 26.6 °2θ, about 10.7 °2θ, about 11.5 °2θ, about 11.7 °2θ, about 12.3 °2θ, about 15.4 °2θ, about 18.8 °2θ, about 23.6 °2θ, and about 26.8 °2θ (±0.2 °2θ; ±0.1 °2θ; or ±0.0 °2θ; Cu Kα1 radiation). In some embodiments, morphic Form D can be characterized by PXRD peaks at about 26.6 °2θ, about 10.7 °2θ, about 11.5 °2θ, about 11.7 °2θ, about 12.3 °2θ, about 13.8 °2θ, about 15.4 °2θ, about 18.8 °2θ, about 23.6 °2θ, and about 26.8 °2θ (±0.2 °2θ; ±0.1 °2θ; or ±0.0 °2θ; Cu Kα1 radiation). In some embodiments, morphic Form D can be characterized by PXRD peaks at about 26.6 °2θ, about 10.7 °2θ, about 11.5 °2θ, about 11.7 °2θ, about 12.3 °2θ, about 13.8 °2θ, about 15.4 °2θ, about 16.4, about 18.8 °2θ, about 23.6 °2θ, and about 26.8 °2θ (±0.2 °2θ; ±0.1 °2θ; or ±0.0 °2θ; Cu Kα1 radiation).

Accordingly, in some embodiments, morphic Form D can be characterized by one, two, three, four, five, six, seven, eight, nine, ten, or eleven PXRD peaks selected from the group consisting of about 10.7 °2θ, 11.5 °2θ, 11.7 °2θ, 12.3 °2θ, 13.8 °2θ, 15.4 °2θ, 16.4 °2θ, 18.8 °2θ, 23.6 °2θ, 26.6 °2θ, and 26.8 °2θ (Cu Kα1 radiation). In some embodiments, morphic Form D can be characterized by one, PXRD peak selected from the group consisting of about 10.7 °2θ, 11.5 °2θ, 11.7 °2θ, 12.3 °2θ, 13.8 °2θ, 15.4 °2θ, 16.4 °2θ, 18.8 °2θ, 23.6 °2θ, 26.6 °2θ, and 26.8 °2θ (Cu Kα1 radiation). In some embodiments, morphic Form D can be characterized by two PXRD peaks selected from the group consisting of about 10.7 °2θ, 11.5 °2θ, 11.7 °2θ, 12.3 °2θ, 13.8 °2θ, 15.4 °2θ, 16.4 °2θ, 18.8 °2θ, 23.6 °2θ, 26.6 °2θ, and 26.8 °2θ (Cu Kα1 radiation). In some embodiments, morphic Form D can be characterized by three PXRD peaks selected from the group consisting of about 10.7 °2θ, 11.5 °2θ, 11.7 °2θ, 12.3 °2θ, 13.8 °2θ, 15.4 °2θ, 16.4 °2θ, 18.8 °2θ, 23.6 °2θ, 26.6 °2θ, and 26.8 °2θ (Cu Kα1 radiation). In some embodiments, morphic Form D can be characterized by four PXRD peaks selected from the group consisting of about 10.7 °2θ, 11.5 °2θ, 11.7 °2θ, 12.3 °2θ, 13.8 °2θ, 15.4 °2θ, 16.4 °2θ, 18.8 °2θ, 23.6 °2θ, 26.6 °2θ, and 26.8 °2θ (Cu Kα1 radiation). In some embodiments, morphic Form D can be characterized by five PXRD peaks selected from the group consisting of about 10.7 °2θ, 11.5 °2θ, 11.7 °2θ, 12.3 °2θ, 13.8 °2θ, 15.4 °2θ, 16.4 °2θ, 18.8 °2θ, 23.6 °2θ, 26.6 °2θ, and 26.8 °2θ (Cu Kα1 radiation). In some embodiments, morphic Form D can be characterized by six PXRD peaks selected from the group consisting of about 10.7 °2θ, 11.5 °2θ, 11.7 °2θ, 12.3 °2θ, 13.8 °2θ, 15.4 °2θ, 16.4 °2θ, 18.8 °2θ, 23.6 °2θ, 26.6 °2θ, and 26.8 °2θ (Cu Kα1 radiation). In some embodiments, morphic Form D can be characterized by seven PXRD peaks selected from the group consisting of about 10.7 °2θ, 11.5 °2θ, 11.7 °2θ, 12.3 °2θ, 13.8 °2θ, 15.4 °2θ, 16.4 °2θ, 18.8 °2θ, 23.6 °2θ, 26.6 °2θ, and 26.8 °2θ (Cu Kα1 radiation). In some embodiments, morphic Form D can be characterized by eight PXRD peaks selected from the group consisting of about 10.7 °2θ, 11.5 °2θ, 11.7 °2θ, 12.3 °2θ, 13.8 °2θ, 15.4 °2θ, 16.4 °2θ, 18.8 °2θ, 23.6 °2θ, 26.6 °2θ, and 26.8 °2θ (Cu Kα1 radiation). In some embodiments, morphic Form D can be characterized by nine PXRD peaks selected from the group consisting of about 10.7 °2θ, 11.5 °2θ, 11.7 °2θ, 12.3 °2θ, 13.8 °2θ, 15.4 °2θ, 16.4 °2θ, 18.8 °2θ, 23.6 °2θ, 26.6 °2θ, and 26.8 °2θ (Cu Kα1 radiation). In some embodiments, morphic Form D can be characterized by ten PXRD peaks selected from the group consisting of about 10.7 °2θ, 11.5 °2θ, 11.7 °2θ, 12.3 °2θ, 13.8 °2θ, 15.4 °2θ, 16.4 °2θ, 18.8 °2θ, 23.6 °2θ, 26.6 °2θ, and 26.8 °2θ (Cu Kα1 radiation). In some embodiments, morphic Form D can be characterized by eleven PXRD peaks selected from the group consisting of about 10.7 °2θ, 11.5 °2θ, 11.7 °2θ, 12.3 °2θ, 13.8 °2θ, 15.4 °2θ, 16.4 °2θ, 18.8 °2θ, 23.6 °2θ, 26.6 °2θ, and 26.8 °2θ (Cu Kα1 radiation).

### Form E

In some embodiments, the disclosure is directed methods of treating and/or preventing a SARS-CoV-2 infection and/or a SARS-CoV-2 infection-associated disease or disorder (e.g. COVID-19) with Compound 1 Form E.

Morphic Form E can be characterized by PXRD peaks at about 10.9 °2θ and/or 26.5 °2θ (±0.2 °2θ; ±0.1 °2θ; or ±0.0 °2θ; Cu Kα1 radiation). Morphic Form E can further be characterized by a PXRD peak at about 14.4 °2θ, 17.8 °2θ, and/or 18.5 °2θ (±0.2 °2θ; ±0.1 °2θ; or ±0.0 °2θ; Cu Kα1 radiation). Morphic Form E can further be characterized by a PXRD peak at about 10.7 °2θ, 12.1 °2θ, 16.2 °2θ, 16.8 °2θ, 22.5 °2θ, 24.5 °2θ, and/or 26.2 °2θ (±0.2 °2θ; ±0.1 °2θ; or ±0.0 °2θ; Cu Kα1 radiation).

In some embodiments, morphic Form E can be characterized by PXRD peaks at about 10.9 °2θ, about 14.4 °2θ, and about 17.8 °2θ (±0.2 °2θ; ±0.1 °2θ; or ±0.0 °2θ; Cu Kα1 radiation). In some embodiments, morphic Form E can be characterized by PXRD peaks at about 14.4 °2θ, about 17.8 °2θ, and about 18.5 °2θ (±0.2 °2θ; ±0.1 °2θ; or ±0.0 °2θ; Cu Kα1 radiation). In some embodiments, morphic Form E can be characterized by PXRD peaks at about 17.8 °2θ, about 18.5 °2θ, and about 26.5 °2θ (±0.2 °2θ; ±0.1 °2θ; or ±0.0 °2θ; Cu Kα1 radiation).

In some embodiments, morphic Form E can be characterized by PXRD peaks at about 10.9 °2θ and about 26.5 °2θ (±0.2 °2θ; ±0.1 °2θ; or ±0.0 °2θ; Cu Kα1 radiation). In some embodiments, morphic Form E can be characterized by PXRD peaks at about 10.9 °2θ, about 14.4 °2θ, and about 26.5 °2θ (±0.2 °2θ; ±0.1 °2θ; or ±0.0 °2θ; Cu Kα1 radiation). In some embodiments, morphic Form E can be characterized by PXRD peaks at about 10.9 °2θ, about 14.4 °2θ, about 17.8 °2θ, and about 26.5 °2θ (±0.2 °2θ; ±0.1 °2θ; or ±0.0 °2θ; Cu Kα1 radiation). In some embodiments, morphic Form E can be characterized by PXRD peaks at about 10.9 °2θ, about 14.4 °2θ, about 17.8 °2θ, about 18.5 °2θ, and about 26.5 °2θ (±0.2 °2θ; ±0.1 °2θ; or ±0.0 °2θ; Cu Kα1 radiation).

In some embodiments, morphic Form E can be characterized by PXRD peaks at about 10.7 °2θ, about 10.9 °2θ, about 14.4 °2θ, about 17.8 °2θ, about 18.5 °2θ, and about 26.5 °2θ (±0.2 °2θ; ±0.1 °2θ; or ±0.0 °2θ; Cu Kα1 radiation). In some embodiments, morphic Form E can be characterized by PXRD peaks at about 10.7 °2θ, about 10.9 °2θ, about 12.1 °2θ, about 14.4 °2θ, about 17.8 °2θ, about 18.5 °2θ, and about 26.5 °2θ (±0.2 °2θ; ±0.1 °2θ; or ±0.0 °2θ; Cu Kα1 radiation). In some embodiments, morphic Form E can be characterized by PXRD peaks at about 10.7 °2θ, about 10.9 °2θ, about 12.1 °2θ, about 14.4 °2θ, about 16.2 °2θ, about 17.8 °2θ, about 18.5 °2θ, and about 26.5 °2θ (±0.2 °2θ; ±0.1 °2θ; or ±0.0 °2θ; Cu Kα1 radiation). In some embodiments, morphic Form E can be characterized by PXRD peaks at about 10.7 °2θ, about 10.9 °2θ, about 12.1 °2θ, about 14.4 °2θ, about 16.2 °2θ, about 16.8 °2θ, about 17.8 °2θ, about 18.5 °2θ, and about 26.5 °2θ (±0.2 °2θ; ±0.1 °2θ; or ±0.0 °2θ; Cu Kα1 radiation). In some embodiments, morphic Form E can be characterized by PXRD peaks at about 10.7 °2θ, about 10.9 °2θ, about 12.1 °2θ, about 14.4 °2θ, about 16.2 °2θ, about 16.8 °2θ, about 17.8 °2θ, about 18.5 °2θ, about 22.5 °2θ, and about 26.5 °2θ (±0.2 °2θ; ±0.1 °2θ; or ±0.0 °2θ; Cu Kα1 radiation). In some embodiments, morphic Form E can be characterized by PXRD peaks at about 10.7 °2θ, about 10.9 °2θ, about 12.1 °2θ, about 14.4 °2θ, about 16.2 °2θ, about 16.8 °2θ, about 17.8 °2θ, about 18.5 °2θ, about 22.5 °2θ, about 24.5 °2θ, and about 26.5 °2θ (±0.2 °2θ; ±0.1 °2θ; or ±0.0 °2θ; Cu Kα1 radiation). In some embodiments, morphic Form E can be characterized by PXRD peaks at about 10.7 °2θ, about 10.9 °2θ, about 12.1 °2θ, about 14.4 °2θ, about 16.2 °2θ, about 16.8 °2θ, about 17.8 °2θ, about 18.5 °2θ, about 22.5 °2θ, about 24.5 °2θ, about 26.2 °2θ, and about 26.5 °2θ (±0.2 °2θ; ±0.1 °2θ; or ±0.0 °2θ; Cu Kα1 radiation).

Accordingly, in some embodiments, morphic Form E can be characterized by one, two, three, four, five, six, seven, eight, nine, ten, eleven, or twelve PXRD peaks selected from about 10.7, 10.9, 12.1, 14.4, 16.2, 16.8, 17.8, 18.5, 22.5, 24.5, 26.2, and 26.5 °2θ (Cu Kα1 radiation). In some embodiments, morphic Form E can be characterized by one PXRD peak selected from about 10.7, 10.9, 12.1, 14.4, 16.2, 16.8, 17.8, 18.5, 22.5, 24.5, 26.2, and 26.5 °2θ (Cu Kα1 radiation). In some embodiments, morphic Form E can be characterized by two PXRD peaks selected from about 10.7, 10.9, 12.1, 14.4, 16.2, 16.8, 17.8, 18.5, 22.5, 24.5, 26.2, and 26.5 °2θ (Cu Kα1 radiation). In some embodiments, morphic Form E can be characterized by three PXRD peaks selected from about 10.7, 10.9, 12.1, 14.4, 16.2, 16.8, 17.8, 18.5, 22.5, 24.5, 26.2, and 26.5 °2θ (Cu Kα1 radiation). In some embodiments, morphic Form E can be characterized by four PXRD peaks selected from about 10.7, 10.9, 12.1, 14.4, 16.2, 16.8, 17.8, 18.5, 22.5, 24.5, 26.2, and 26.5 °2θ (Cu Kα1 radiation). In some embodiments, morphic Form E can be characterized by five PXRD peaks selected from about 10.7, 10.9, 12.1, 14.4, 16.2, 16.8, 17.8, 18.5, 22.5, 24.5, 26.2, and 26.5 °2θ (Cu Kα1 radiation). In some embodiments, morphic Form E can be characterized by six PXRD peaks selected from about 10.7, 10.9, 12.1, 14.4, 16.2, 16.8, 17.8, 18.5, 22.5, 24.5, 26.2, and 26.5 °2θ (Cu Kα1 radiation). In some embodiments, morphic Form E can be characterized by seven PXRD peaks selected from about 10.7, 10.9, 12.1, 14.4, 16.2, 16.8, 17.8, 18.5, 22.5, 24.5, 26.2, and 26.5 °2θ (Cu Kα1 radiation). In some embodiments, morphic Form E can be characterized by eight PXRD peaks selected from about 10.7, 10.9, 12.1, 14.4, 16.2, 16.8, 17.8, 18.5, 22.5, 24.5, 26.2, and 26.5 °2θ (Cu Kα1 radiation). In some embodiments, morphic Form E can be characterized by nine PXRD peaks selected from about 10.7, 10.9, 12.1, 14.4, 16.2, 16.8, 17.8, 18.5, 22.5, 24.5, 26.2, and 26.5 °2θ (Cu Kα1 radiation). In some embodiments, morphic Form E can be characterized by ten PXRD peaks selected from about 10.7, 10.9, 12.1, 14.4, 16.2, 16.8, 17.8, 18.5, 22.5, 24.5, 26.2, and 26.5 °2θ (Cu Kα1 radiation). In some embodiments, morphic Form E can be characterized by eleven PXRD peaks selected from about 10.7, 10.9, 12.1, 14.4, 16.2, 16.8, 17.8, 18.5, 22.5, 24.5, 26.2, and 26.5 °2θ (Cu Kα1 radiation). In some embodiments, morphic Form E can be characterized by twelve PXRD peaks selected from about 10.7, 10.9, 12.1, 14.4, 16.2, 16.8, 17.8, 18.5, 22.5, 24.5, 26.2, and 26.5 °2θ (Cu Kα1 radiation).

### Form F

In some embodiments, the disclosure is directed methods of treating and/or preventing a SARS-CoV-2 infection and/or a SARS-CoV-2 infection-associated disease or disorder (e.g. COVID-19) with Compound 1 Form F.

Morphic Form F can be characterized by PXRD peaks at about 15.0 and/or 22.8 °2θ (Cu Kα1 radiation). Form F can further be characterized by PXRD peaks at about 7.1 °2θ, 11.3 °2θ, 12.9 °2θ, 14.3 °2θ, 17.4 °2θ, 19.7 °2θ, and/or 23.2 °2θ (Cu Kα1 radiation).

Form F can further be characterized by PXRD peaks at about 8.4 °2θ, 13.7 °2θ, 18.7 °2θ, 19.5 °2θ, 20.5 °2θ, 24.4 °2θ and/or 28.9 °2θ (Cu Kα1 radiation).

In some embodiments, morphic Form F can be characterized by PXRD peaks at about 7.1 °2θ, about 11.3 °2θ, and about 12.9 °2θ (±0.2 °2θ; ±0.1 °2θ; or ±0.0 °2θ; Cu Kα1 radiation). In some embodiments, morphic Form F can be characterized by PXRD peaks at about 11.3 °2θ, about 12.9 °2θ, and about 14.3 °2θ (±0.2 °2θ; ±0.1 °2θ; or ±0.0 °2θ; Cu Kα1 radiation). In some embodiments, morphic Form F can be characterized by PXRD peaks at about 12.9 °2θ, about 14.3 °2θ, and about 15.0 °2θ (±0.2 °2θ; ±0.1 °2θ; or ±0.0 °2θ; Cu Kα1 radiation). In some embodiments, morphic Form F can be characterized by PXRD peaks at about 14.3 °2θ, about 15.0 °2θ, and about 17.4 °2θ (±0.2 °2θ; ±0.1 °2θ; or ±0.0 °2θ; Cu Kα1 radiation). In some embodiments, morphic Form F can be characterized by PXRD peaks at about 15.0 °2θ, about 17.4 °2θ, and about 19.7 °2θ (±0.2 °2θ; ±0.1 °2θ; or ±0.0 °2θ; Cu Kα1 radiation). In some embodiments, morphic Form F can be characterized by PXRD peaks at about 17.4 °2θ, about 19.7 °2θ, and about 22.8 °2θ (±0.2 °2θ; ±0.1 °2θ; or ±0.0 °2θ; Cu Kα1 radiation). In some embodiments, morphic Form F can be characterized by PXRD peaks at about 19.7 °2θ, about 22.8 °2θ, and about 23.2 °2θ (±0.2 °2θ; ±0.1 °2θ; or ±0.0 °2θ; Cu Kα1 radiation).

In some embodiments, morphic Form F can be characterized by PXRD peaks at about 15.0 °2θ and about 22.8 °2θ (±0.2 °2θ; ±0.1 °2θ; or ±0.0 °2θ; Cu Kα1 radiation). In some embodiments, morphic Form F can be characterized by PXRD peaks at about 7.1 °2θ, about 15.0 °2θ and about 22.8 °2θ (±0.2 °2θ; ±0.1 °2θ; or ±0.0 °2θ; Cu Kα1 radiation). In some embodiments, morphic Form F can be characterized by PXRD peaks at about 7.1 °2θ, about 11.3 °2θ, about 15.0 °2θ and about 22.8 °2θ (±0.2 °2θ; ±0.1 °2θ; or ±0.0 °2θ; Cu Kα1 radiation). In some embodiments, morphic Form F can be characterized by PXRD peaks at about 7.1 °2θ, about 11.3 °2θ, about 12.9 °2θ, about 15.0 °2θ and about 22.8 °2θ (±0.2 °2θ; ±0.1 °2θ; or ±0.0 °2θ; Cu Kα1 radiation). In some embodiments, morphic Form F can be characterized by PXRD peaks at about 7.1 °2θ, about 11.3 °2θ, about 12.9 °2θ, about 14.3 °2θ, about 15.0 °2θ and about 22.8 °2θ (±0.2 °2θ; ±0.1 °2θ; or ±0.0 °2θ; Cu Kα1 radiation). In some embodiments, morphic Form F can be characterized by PXRD peaks at about 7.1 °2θ, about 11.3 °2θ, about 12.9 °2θ, about 14.3 °2θ, about 15.0 °2θ, about 17.4 °2θ, and about 22.8 °2θ (±0.2 °2θ; ±0.1 °2θ; or ±0.0 °2θ; Cu Kα1 radiation). In some embodiments, morphic Form F can be characterized by PXRD peaks at about 7.1 °2θ, about 11.3 °2θ, about 12.9 °2θ, about 14.3 °2θ, about 15.0 °2θ, about 17.4 °2θ, about 19.7 °2θ, and about 22.8 °2θ (±0.2 °2θ; ±0.1 °2θ; or ±0.0 °2θ; Cu Kα1 radiation). In some embodiments, morphic Form F can be characterized by PXRD peaks at about 7.1 °2θ, about 11.3 °2θ, about 12.9 °2θ, about 14.3 °2θ, about 15.0 °2θ, about 17.4 °2θ, about 19.7 °2θ, about 22.8 °2θ, and about 23.2 °2θ (±0.2 °2θ; ±0.1 °2θ; or ±0.0 °2θ; Cu Kα1 radiation).

In some embodiments, morphic Form F can be characterized by PXRD peaks at about 7.1 °2θ, about 8.4 °2θ, about 11.3 °2θ, about 12.9 °2θ, about 14.3 °2θ, about 15.0 °2θ, about 17.4 °2θ, about 19.7 °2θ, about 22.8 °2θ, and about 23.2 °2θ (±0.2 °2θ; ±0.1 °2θ; or ±0.0 °2θ; Cu Kα1 radiation). In some embodiments, morphic Form F can be characterized by PXRD peaks at about 7.1 °2θ, about 8.4 °2θ, about 11.3 °2θ, about 12.9 °2θ, about 13.7 °2θ, about 14.3 °2θ, about 15.0 °2θ, about 17.4 °2θ, about 19.7 °2θ, about 22.8 °2θ, and about 23.2 °2θ (±0.2 °2θ; ±0.1 °2θ; or ±0.0 °2θ; Cu Kα1 radiation). In some embodiments, morphic Form F can be characterized by PXRD peaks at about 7.1 °2θ, about 8.4 °2θ, about 11.3 °2θ, about 12.9 °2θ, about 13.7 °2θ, about 14.3 °2θ, about 15.0 °2θ, about 17.4 °2θ, about 18.7 °2θ, about 19.7 °2θ, about 22.8 °2θ, and about 23.2 °2θ (±0.2 °2θ; ±0.1 °2θ; or ±0.0 °2θ; Cu Kα1 radiation). In some embodiments, morphic Form F can be characterized by PXRD peaks at about 7.1 °2θ, about 8.4 °2θ, about 11.3 °2θ, about 12.9 °2θ, about 13.7 °2θ, about 14.3 °2θ, about 15.0 °2θ, about 17.4 °2θ, about 18.7 °2θ, about 19.5 °2θ, about 19.7 °2θ, about 22.8 °2θ, and about 23.2 °2θ (±0.2 °2θ; ±0.1 °2θ; or ±0.0 °2θ; Cu Kα1 radiation). In some embodiments, morphic Form F can be characterized by PXRD peaks at about 7.1 °2θ, about 8.4 °2θ, about 11.3 °2θ, about 12.9 °2θ, about 13.7 °2θ, about 14.3 °2θ, about 15.0 °2θ, about 17.4 °2θ, about 18.7 °2θ, about 19.5 °2θ, about 19.7 °2θ, about 20.5 °2θ, about 22.8 °2θ, and about 23.2 °2θ (±0.2 °2θ; ±0.1 °2θ; or ±0.0 °2θ; Cu Kα1 radiation). In some embodiments, morphic Form F can be characterized by PXRD peaks at about 7.1 °2θ, about 8.4 °2θ, about 11.3 °2θ, about 12.9 °2θ, about 13.7 °2θ, about 14.3 °2θ, about 15.0 °2θ, about 17.4 °2θ, about 18.7 °2θ, about 19.5 °2θ, about 19.7 °2θ, about 20.5 °2θ, about 22.8 °2θ, about 23.2 °2θ, and about 24.4 °2θ (±0.2 °2θ; ±0.1 °2θ; or ±0.0 °2θ; Cu Kα1 radiation). In some embodiments, morphic Form F can be characterized by PXRD peaks at about 7.1 °2θ, about 8.4 °2θ, about 11.3 °2θ, about 12.9 °2θ, about 13.7 °2θ, about 14.3 °2θ, about 15.0 °2θ, about 17.4 °2θ, about 18.7 °2θ, about 19.5 °2θ, about 19.7 °2θ, about 20.5 °2θ, about 22.8 °2θ, about 23.2 °2θ, about 24.4 °2θ, and about 28.9 °2θ (±0.2 °2θ; ±0.1 °2θ; or ±0.0 °2θ; Cu Kα1 radiation).

Accordingly, in some embodiments, morphic Form F is characterized by one, two, three, four, five, six, seven, eight, nine, ten, eleven, twelve, thirteen, fourteen, fifteen or sixteen peaks selected from about 7.1, 8.4, 11.3, 12.9, 13.7, 14.3, 15.0, 17.4, 18.7, 19.5, 19.7, 20.5, 22.8, 23.2, 24.4, and 28.9 °2θ (Cu Kα1 radiation).

In some embodiments, morphic Form F is characterized by one peak selected from about 7.1 °2θ, 8.4 °2θ, 11.3 °2θ, 12.9 °2θ, 13.7 °2θ, 14.3 °2θ, 15.0 °2θ, 17.4 °2θ, 18.7 °2θ, 19.5 °2θ, 19.7 °2θ, 20.5 °2θ, 22.8 °2θ, 23.2 °2θ, 24.4 °2θ, and 28.9 °2θ (±0.2 °2θ; ±0.1 °2θ; or ±0.0 °2θ; Cu Kα1 radiation). In some embodiments, morphic Form F is characterized by two peaks selected from about 7.1 °2θ, 8.4 °2θ, 11.3 °2θ, 12.9 °2θ, 13.7 °2θ, 14.3 °2θ, 15.0 °2θ, 17.4 °2θ, 18.7 °2θ, 19.5 °2θ, 19.7 °2θ, 20.5 °2θ, 22.8 °2θ, 23.2 °2θ, 24.4 °2θ, and 28.9 °2θ (±0.2 °2θ; ±0.1 °2θ; or ±0.0 °2θ; Cu Kα1 radiation). In some embodiments, morphic Form F is characterized by three peaks selected from about 7.1 °2θ, 8.4 °2θ, 11.3 °2θ, 12.9 °2θ, 13.7 °2θ, 14.3 °2θ, 15.0 °2θ, 17.4 °2θ, 18.7 °2θ, 19.5 °2θ, 19.7 °2θ, 20.5 °2θ, 22.8 °2θ, 23.2 °2θ, 24.4 °2θ, and 28.9 °2θ (±0.2 °2θ; ±0.1 °2θ; or ±0.0 °2θ; Cu Kα1 radiation). In some embodiments, morphic Form F is characterized by four peaks selected from about 7.1 °2θ, 8.4 °2θ, 11.3 °2θ, 12.9 °2θ, 13.7 °2θ, 14.3 °2θ, 15.0 °2θ, 17.4 °2θ, 18.7 °2θ, 19.5 °2θ, 19.7 °2θ, 20.5 °2θ, 22.8 °2θ, 23.2 °2θ, 24.4 °2θ, and 28.9 °2θ (±0.2 °2θ; ±0.1 °2θ; or ±0.0 °2θ; Cu Kα1 radiation). In some embodiments, morphic Form F is characterized by five peaks selected from about 7.1 °2θ, 8.4 °2θ, 11.3 °2θ, 12.9 °2θ, 13.7 °2θ, 14.3 °2θ, 15.0 °2θ, 17.4 °2θ, 18.7 °2θ, 19.5 °2θ, 19.7 °2θ, 20.5 °2θ, 22.8 °2θ, 23.2 °2θ, 24.4 °2θ, and 28.9 °2θ (±0.2 °2θ; ±0.1 °2θ; or ±0.0 °2θ; Cu Kα1 radiation). In some embodiments, morphic Form F is characterized by six peaks selected from about 7.1 °2θ, 8.4 °2θ, 11.3 °2θ, 12.9 °2θ, 13.7 °2θ, 14.3 °2θ, 15.0 °2θ, 17.4 °2θ, 18.7 °2θ, 19.5 °2θ, 19.7 °2θ, 20.5 °2θ, 22.8 °2θ, 23.2 °2θ, 24.4 °2θ, and 28.9 °2θ (±0.2 °2θ; ±0.1 °2θ; or ±0.0 °2θ; Cu Kα1 radiation). In some embodiments, morphic Form F is characterized by seven peaks selected from about 7.1 °2θ, 8.4 °2θ, 11.3 °2θ, 12.9 °2θ, 13.7 °2θ, 14.3 °2θ, 15.0 °2θ, 17.4 °2θ, 18.7 °2θ, 19.5 °2θ, 19.7 °2θ, 20.5 °2θ, 22.8 °2θ, 23.2 °2θ, 24.4 °2θ, and 28.9 °2θ (±0.2 °2θ; ±0.1 °2θ; or ±0.0 °2θ; Cu Kα1 radiation). In some embodiments, morphic Form F is characterized by eight peaks selected from about 7.1 °2θ, 8.4 °2θ, 11.3 °2θ, 12.9 °2θ, 13.7 °2θ, 14.3 °2θ, 15.0 °2θ, 17.4 °2θ, 18.7 °2θ, 19.5 °2θ, 19.7 °2θ, 20.5 °2θ, 22.8 °2θ, 23.2 °2θ, 24.4 °2θ, and 28.9 °2θ (±0.2 °2θ; ±0.1 °2θ; or ±0.0 °2θ; Cu Kα1 radiation). In some embodiments, morphic Form F is characterized by nine peaks selected from about 7.1 °2θ, 8.4 °2θ, 11.3 °2θ, 12.9 °2θ, 13.7 °2θ, 14.3 °2θ, 15.0 °2θ, 17.4 °2θ, 18.7 °2θ, 19.5 °2θ, 19.7 °2θ, 20.5 °2θ, 22.8 °2θ, 23.2 °2θ, 24.4 °2θ, and 28.9 °2θ (±0.2 °2θ; ±0.1 °2θ; or ±0.0 °2θ; Cu Kα1 radiation). In some embodiments, morphic Form F is characterized by ten peaks selected from about 7.1 °2θ, 8.4 °2θ, 11.3 °2θ, 12.9 °2θ, 13.7 °2θ, 14.3 °2θ, 15.0 °2θ, 17.4 °2θ, 18.7 °2θ, 19.5 °2θ, 19.7 °2θ, 20.5 °2θ, 22.8 °2θ, 23.2 °2θ, 24.4 °2θ, and 28.9 °2θ (±0.2 °2θ; ±0.1 °2θ; or ±0.0 °2θ; Cu Kα1 radiation). In some embodiments, morphic Form F is characterized by eleven peaks selected from about 7.1 °2θ, 8.4 °2θ, 11.3 °2θ, 12.9 °2θ, 13.7 °2θ, 14.3 °2θ, 15.0 °2θ, 17.4 °2θ, 18.7 °2θ, 19.5 °2θ, 19.7 °2θ, 20.5 °2θ, 22.8 °2θ, 23.2 °2θ, 24.4 °2θ, and 28.9 °2θ (±0.2 °2θ; ±0.1 °2θ; or ±0.0 °2θ; Cu Kα1 radiation). In some embodiments, morphic Form F is characterized by twelve peaks selected from about 7.1 °2θ, 8.4 °2θ, 11.3 °2θ, 12.9 °2θ, 13.7 °2θ, 14.3 °2θ, 15.0 °2θ, 17.4 °2θ, 18.7 °2θ, 19.5 °2θ, 19.7 °2θ, 20.5 °2θ, 22.8 °2θ, 23.2 °2θ, 24.4 °2θ, and 28.9 °2θ (±0.2 °2θ; ±0.1 °2θ; or ±0.0 °2θ; Cu Kα1 radiation). In some embodiments, morphic Form F is characterized by thirteen peaks selected from about 7.1 °2θ, 8.4 °2θ, 11.3 °2θ, 12.9 °2θ, 13.7 °2θ, 14.3 °2θ, 15.0 °2θ, 17.4 °2θ, 18.7 °2θ, 19.5 °2θ, 19.7 °2θ, 20.5 °2θ, 22.8 °2θ, 23.2 °2θ, 24.4 °2θ, and 28.9 °2θ (±0.2 °2θ; ±0.1 °2θ; or ±0.0 °2θ; Cu Kα1 radiation). In some embodiments, morphic Form F is characterized by fourteen peaks selected from about 7.1 °2θ, 8.4 °2θ, 11.3 °2θ, 12.9 °2θ, 13.7 °2θ, 14.3 °2θ, 15.0 °2θ, 17.4 °2θ, 18.7 °2θ, 19.5 °2θ, 19.7 °2θ, 20.5 °2θ, 22.8 °2θ, 23.2 °2θ, 24.4 °2θ, and 28.9 °2θ (±0.2 °2θ; ±0.1 °2θ; or ±0.0 °2θ; Cu Kα1 radiation). In some embodiments, morphic Form F is characterized by fifteen peaks selected from about 7.1 °2θ, 8.4 °2θ, 11.3 °2θ, 12.9 °2θ, 13.7 °2θ, 14.3 °2θ, 15.0 °2θ, 17.4 °2θ, 18.7 °2θ, 19.5 °2θ, 19.7 °2θ, 20.5 °2θ, 22.8 °2θ, 23.2 °2θ, 24.4 °2θ, and 28.9 °2θ (±0.2 °2θ; ±0.1 °2θ; or ±0.0 °2θ; Cu Kα1 radiation). In some embodiments, morphic Form F is characterized by sixteen peaks selected from about 7.1 °2θ, 8.4 °2θ, 11.3 °2θ, 12.9 °2θ, 13.7 °2θ, 14.3 °2θ, 15.0 °2θ, 17.4 °2θ, 18.7 °2θ, 19.5 °2θ, 19.7 °2θ, 20.5 °2θ, 22.8 °2θ, 23.2 °2θ, 24.4 °2θ, and 28.9 °2θ (±0.2 °2θ; ±0.1 °2θ; or ±0.0 °2θ; Cu Kα1 radiation).

### Form G

In some embodiments, the disclosure is directed methods of treating and/or preventing a SARS-CoV-2 infection and/or a SARS-CoV-2 infection-associated disease or disorder (e.g. COVID-19) with Compound 1 Form G.

Morphic Form G can be characterized by a PXRD peak at about 22.8 °2θ (Cu Kα1 radiation). Morphic Form G can further be characterized by PXRD peaks at about 7.1 °2θ, 11.3 °2θ, 12.9 °2θ, 14.3 °2θ, 15.0 °2θ, 23.2 °2θ, and/or 24.4 °2θ (Cu Kα1 radiation). Morphic Form G can further be characterized by PXRD peaks at about 8.4 °2θ, 13.7 °2θ, 17.4 °2θ, 19.5 °2θ, 19.7 °2θ, 20.5 °2θ, and/or 28.9 °2θ (Cu Kα1 radiation). In some embodiments, morphic Form G can be characterized by PXRD peaks at about 22.8 °2θ and about 7.1 °2θ (±0.2 °2θ; ±0.1 °2θ; or ±0.0 °2θ; Cu Kα1 radiation). In some embodiments, morphic Form G can be characterized by PXRD peaks at about 22.8 °2θ, about 7.1 °2θ, and about 11.3 °2θ (±0.2 °2θ; ±0.1 °2θ; or ±0.0 °2θ; Cu Kα1 radiation). In some embodiments, morphic Form G can be characterized by PXRD peaks at about 22.8 °2θ, about 7.1 °2θ, about 11.3 °2θ, and about 12.9 °2θ (±0.2 °2θ; ±0.1 °2θ; or ±0.0 °2θ; Cu Kα1 radiation). In some embodiments, morphic Form G can be characterized by PXRD peaks at about 22.8 °2θ, about 7.1 °2θ, about 11.3 °2θ, about 12.9 °2θ, and about 14.3 °2θ (±0.2 °2θ; ±0.1 °2θ; or ±0.0 °2θ; Cu Kα1 radiation). In some embodiments, morphic Form G can be characterized by PXRD peaks at about 22.8 °2θ, about 7.1 °2θ, about 11.3 °2θ, about 12.9 °2θ, about 14.3 °2θ, and about 15.0 °2θ (±0.2 °2θ; ±0.1 °2θ; or ±0.0 °2θ; Cu Kα1 radiation). In some embodiments, morphic Form G can be characterized by PXRD peaks at about 22.8 °2θ, about 7.1 °2θ, about 11.3 °2θ, about 12.9 °2θ, about 14.3 °2θ, about 15.0 °2θ, and about 23.2 °2θ (±0.2 °2θ; ±0.1 °2θ; or ±0.0 °2θ; Cu Kα1 radiation). In some embodiments, morphic Form G can be characterized by PXRD peaks at about 22.8 °2θ, about 7.1 °2θ, about 11.3 °2θ, about 12.9 °2θ, about 14.3 °2θ, about 15.0 °2θ, about 23.2 °2θ, and about 24.4 °2θ (±0.2 °2θ; ±0.1 °2θ; or ±0.0 °2θ; Cu Kα1 radiation).

In some embodiments, morphic Form G can be characterized by PXRD peaks at about 22.8 °2θ, about 7.1 °2θ, about 8.4 °2θ, about 11.3 °2θ, about 12.9 °2θ, about 14.3 °2θ, about 15.0 °2θ, about 23.2 °2θ, and about 24.4 °2θ (±0.2 °2θ; ±0.1 °2θ; or ±0.0 °2θ; Cu Kα1 radiation). In some embodiments, morphic Form G can be characterized by PXRD peaks at about 22.8 °2θ, about 7.1 °2θ, about 8.4 °2θ, about 11.3 °2θ, about 12.9 °2θ, about 13.7 °2θ, about 14.3 °2θ, about 15.0 °2θ, about 23.2 °2θ, and about 24.4 °2θ (±0.2 °2θ; ±0.1 °2θ; or ±0.0 °2θ; Cu Kα1 radiation). In some embodiments, morphic Form G can be characterized by PXRD peaks at about 22.8 °2θ, about 7.1 °2θ, about 8.4 °2θ, about 11.3 °2θ, about 12.9 °2θ, about 13.7 °2θ, about 14.3 °2θ, about 15.0 °2θ, about 17.4 °2θ, about 23.2 °2θ, and about 24.4 °2θ (±0.2 °2θ; ±0.1 °2θ; or ±0.0 °2θ; Cu Kα1 radiation). In some embodiments, morphic Form G can be characterized by PXRD peaks at about 22.8 °2θ, about 7.1 °2θ, about 8.4 °2θ, about 11.3 °2θ, about 12.9 °2θ, about 13.7 °2θ, about 14.3 °2θ, about 15.0 °2θ, about 17.4 °2θ, about 19.5 °2θ, about 23.2 °2θ, and about 24.4 °2θ (±0.2 °2θ; ±0.1 °2θ; or ±0.0 °2θ; Cu Kα1 radiation). In some embodiments, morphic Form G can be characterized by PXRD peaks at about 22.8 °2θ, about 7.1 °2θ, about 8.4 °2θ, about 11.3 °2θ, about 12.9 °2θ, about 13.7 °2θ, about 14.3 °2θ, about 15.0 °2θ, about 17.4 °2θ, about 19.5 °2θ, about 19.7 °2θ, about 23.2 °2θ, and about 24.4 °2θ (±0.2 °2θ; ±0.1 °2θ; or ±0.0 °2θ; Cu Kα1 radiation). In some embodiments, morphic Form G can be characterized by PXRD peaks at about 22.8 °2θ, about 7.1 °2θ, about 8.4 °2θ, about 11.3 °2θ, about 12.9 °2θ, about 13.7 °2θ, about 14.3 °2θ, about 15.0 °2θ, about 17.4 °2θ, about 19.5 °2θ, about 19.7 °2θ, about 20.5 °2θ, about 23.2 °2θ, and about 24.4 °2θ (±0.2 °2θ; ±0.1 °2θ; or ±0.0 °2θ; Cu Kα1 radiation). In some embodiments, morphic Form G can be characterized by PXRD peaks at about 22.8 °2θ, about 7.1 °2θ, about 8.4 °2θ, about 11.3 °2θ, about 12.9 °2θ, about 13.7 °2θ, about 14.3 °2θ, about 15.0 °2θ, about 17.4 °2θ, about 19.5 °2θ, about 19.7 °2θ, about 20.5 °2θ, about 23.2 °2θ, about 24.4 °2θ, and about 28.9 °2θ (±0.2 °2θ; ±0.1 °2θ; or ±0.0 °2θ; Cu Kα1 radiation).

Accordingly, in some embodiments, morphic Form G is characterized by one, two, three, four, five, six, seven, eight, nine, ten, eleven, twelve, thirteen, or fourteen PXRD peaks selected from about 7.1, 8.4, 11.3, 12.9, 13.7, 14.3, 15.0, 17.4, 19.5, 19.7, 20.5, 22.8, and 23.2 °2θ (Cu Kα1 radiation). In some embodiments, morphic Form G is characterized by one PXRD peak selected from about 7.1, 8.4, 11.3, 12.9, 13.7, 14.3, 15.0, 17.4, 19.5, 19.7, 20.5, 22.8, and 23.2 °2θ (Cu Kα1 radiation). In some embodiments, morphic Form G is characterized by two PXRD peaks selected from about 7.1, 8.4, 11.3, 12.9, 13.7, 14.3, 15.0, 17.4, 19.5, 19.7, 20.5, 22.8, and 23.2 °2θ (Cu Kα1 radiation). In some embodiments, morphic Form G is characterized by three PXRD peaks selected from about 7.1, 8.4, 11.3, 12.9, 13.7, 14.3, 15.0, 17.4, 19.5, 19.7, 20.5, 22.8, and 23.2 °2θ (Cu Kα1 radiation). In some embodiments, morphic Form G is characterized by four PXRD peaks selected from about 7.1, 8.4, 11.3, 12.9, 13.7, 14.3, 15.0, 17.4, 19.5, 19.7, 20.5, 22.8, and 23.2 °2θ (Cu Kα1 radiation). In some embodiments, morphic Form G is characterized by five PXRD peaks selected from about 7.1, 8.4, 11.3, 12.9, 13.7, 14.3, 15.0, 17.4, 19.5, 19.7, 20.5, 22.8, and 23.2 °2θ (Cu Kα1 radiation). In some embodiments, morphic Form G is characterized by six PXRD peaks selected from about 7.1, 8.4, 11.3, 12.9, 13.7, 14.3, 15.0, 17.4, 19.5, 19.7, 20.5, 22.8, and 23.2 °2θ (Cu Kα1 radiation). In some embodiments, morphic Form G is characterized by seven PXRD peaks selected from about 7.1, 8.4, 11.3, 12.9, 13.7, 14.3, 15.0, 17.4, 19.5, 19.7, 20.5, 22.8, and 23.2 °2θ (Cu Kα1 radiation). In some embodiments, morphic Form G is characterized by eight PXRD peaks selected from about 7.1, 8.4, 11.3, 12.9, 13.7, 14.3, 15.0, 17.4, 19.5, 19.7, 20.5, 22.8, and 23.2 °2θ (Cu Kα1 radiation). In some embodiments, morphic Form G is characterized by nine PXRD peaks selected from about 7.1, 8.4, 11.3, 12.9, 13.7, 14.3, 15.0, 17.4, 19.5, 19.7, 20.5, 22.8, and 23.2 °2θ (Cu Kα1 radiation). In some embodiments, morphic Form G is characterized by ten PXRD peaks selected from about 7.1, 8.4, 11.3, 12.9, 13.7, 14.3, 15.0, 17.4, 19.5, 19.7, 20.5, 22.8, and 23.2 °2θ (Cu Kα1 radiation). In some embodiments, morphic Form G is characterized by eleven PXRD peaks selected from about 7.1, 8.4, 11.3, 12.9, 13.7, 14.3, 15.0, 17.4, 19.5, 19.7, 20.5, 22.8, and 23.2 °2θ (Cu Kα1 radiation). In some embodiments, morphic Form G is characterized by twelve PXRD peaks selected from about 7.1, 8.4, 11.3, 12.9, 13.7, 14.3, 15.0, 17.4, 19.5, 19.7, 20.5, 22.8, and 23.2 °2θ (Cu Kα1 radiation). In some embodiments, morphic Form G is characterized by thirteen PXRD peaks selected from about 7.1, 8.4, 11.3, 12.9, 13.7, 14.3, 15.0, 17.4, 19.5, 19.7, 20.5, 22.8, and 23.2 °2θ (Cu Kα1 radiation). In some embodiments, morphic Form G is characterized by fourteen PXRD peaks selected from about 7.1, 8.4, 11.3, 12.9, 13.7, 14.3, 15.0, 17.4, 19.5, 19.7, 20.5, 22.8, and 23.2 °2θ (Cu Kα1 radiation).

### Pharmaceutical Compositions and Methods of Treatment

As set forth above, provided herein are pharmaceutical compositions comprising compounds of the disclosure (e.g., Formula I, Formula IA, Formula IB or Formula II) or pharmaceutically acceptable salts thereof. In some embodiments, the present disclosure provides pharmaceutical compositions comprising compounds of Formula I, Formula IA, Formula IB or Formula II or pharmaceutically acceptable salts thereof and a pharmaceutically acceptable carrier and/or diluent. In some embodiments the present disclosure provides compounds of Formula I, Formula IA, Formula IB or Formula II formulated as a pharmaceutical composition. In some embodiments, compounds of Formula I, Formula IA, Formula IB or Formula II is formulated as a tablet. In another embodiment, compounds of Formula I, Formula IA, Formula IB or Formula II is formulated as a suspension.

Techniques for formulation and administration of the disclosed compounds can be found in Remington: the Science and Practice of Pharmacy, 22^{nd} edition, Pharmaceutical Press (2012).

In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

The compounds of the disclosure, e.g., compounds of Formula I, Formula IA, Formula IB or Formula II or pharmaceutically acceptable salts thereof described herein may be combined with a pharmaceutically acceptable carrier according to conventional pharmaceutical compounding techniques. Furthermore, the carrier may take a wide variety of forms depending on the form of the preparation desired for administration, e.g. oral, nasal, rectal, vaginal, parenteral (including intravenous injections or infusions). In preparing compositions for oral dosage form any of the usual pharmaceutical media may be employed. Usual pharmaceutical media include, for example, water, glycols, oils, alcohols, flavoring agents, preservatives, coloring agents, and the like in the case of oral liquid preparations (such as for example, suspensions, solutions, emulsions and elixirs); aerosols; or carriers such as starches, sugars, microcrystalline cellulose, diluents, granulating agents, lubricants, binders, disintegrating agents and the like, in the case of oral solid preparations (such as for example, powders, capsules, and tablets).

In another embodiment, the disclosure provides a method for the therapeutic and/or prophylactic treatment of viral infection in a subject, e.g., an immunodeficient subject, the method comprising administering any one of the compounds of Formula I, Formula IA, Formula IB or Formula II or pharmaceutically acceptable salt thereof. In some embodiments, the salt has a purity of equal to or greater than 91% w/w, e.g., having less than or equal to 9% w/w of impurities, to the subject.

Pharmaceutical compositions comprising the compounds of the present disclosure (e.g., compounds of Formula I, Formula IA, Formula IB or Formula II) may be formulated to have any concentration desired. In some embodiments, the composition is formulated such that it comprises at least a therapeutically effective amount.

Pharmaceutical compositions include those suitable for oral, sublingual, nasal, rectal, vaginal, topical, buccal and parenteral (including subcutaneous, intramuscular, and intravenous) administration, although the most suitable route will depend on the nature and severity of the condition being treated. The compositions may be conveniently presented in unit dosage form, and prepared by any of the methods well known in the art of pharmacy. In certain embodiments, the pharmaceutical composition is formulated for oral administration in the form of a pill, capsule, lozenge or tablet. In other embodiments, the pharmaceutical composition is in the form of a suspension.

When the compounds of the present disclosure are administered as pharmaceuticals to mammals, e.g., humans, they can be given per se or as a pharmaceutical composition containing, for example, about 0.1% to 99.9%, about 0.2 to 98%, about 0.3% to 97%, about 0.4% to 96%, or about 0.5 to 95% of active ingredient in combination with a pharmaceutically acceptable carrier. In some embodiments pharmaceutical composition containing about 0.5% to 90% of active ingredient in combination with a pharmaceutically acceptable carrier is suitable for administration to mammals, e.g., humans. Some embodiments of the present disclosure provide preparation of a pharmaceutical composition comprising about 0.1% to 99.9%, about 0.2 to 98%, about 0.3% to 97%, about 0.4% to 96%, or about 0.5 to 95% of the compounds of Formula I, Formula IA, Formula IB or Formula II or pharmaceutically acceptable salts thereof, e.g., any one of the Compounds in Table 1 or pharmaceutically acceptable salt thereof, for use in treating, preventing, or prophylaxis of viral infections or viral infection associated disorders. The present disclosure provides use of about 0.1% to 99.9%, about 0.2 to 98%, about 0.3% to 97%, about 0.4% to 96%, or about 0.5 to 95% of the compounds of Formula I, Formula IA, Formula IB or Formula II or pharmaceutically acceptable salts thereof for the manufacture of a medicament containing effective amounts of the compound for use in treating, preventing, or prophylaxis of viral infections and viral infection associated diseases.

For any compound, the therapeutically effective amount of a compound or composition can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans.

Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as, for example, peppermint, methyl salicylate, or orange flavoring.

The compounds of Formula I, Formula IA, Formula IB or Formula II or pharmaceutically acceptable salts thereof are formulated as a pharmaceutical composition or are used in the manufacture of a medicament for the treatment of a SARS-CoV-2 infection and/or SARS-CoV-2 infection associated disease and/or disorder. The composition and/or the medicament of the compounds of Formula I, Formula IA, Formula IB or Formula II or pharmaceutically acceptable salts thereof are formulated as a tablet or suspension. Tablets of the compounds of Formula I, Formula IA, Formula IB or Formula II or pharmaceutically acceptable salts thereof are formulated comprising pharmacologically acceptable buffers, excipients, carriers, including emulsifiers, enhancers (e.g., absorption enhancers), disintegrants (e.g., Polyvinylpolypyrrolidone (polyvinyl polypyrrolidone, PVPP, crospovidone, crospolividone or E1202), which is a highly cross-linked modification of polyvinylpyrrolidone (PVP)), and/or polymers disclosed in the present disclosure and well-known in the art.

In some embodiments, the present disclosure provides tablet formulation of the compounds of Formula I, Formula IA, Formula IB or Formula II or pharmaceutically acceptable salts thereof for use in prophylactic treatment or prevention viral infection and/or viral associated disease or disorder. The present disclosure provides tablet formulation of the compounds of Formula I, Formula IA, Formula IB or Formula II or pharmaceutically acceptable salts thereof for use in treating subjects in need of such treatment including but not limited to immunodeficient subjects, or pre- or post-organ and/or tissue transplantation subjects. The present disclosure provides the compounds of Formula I, Formula IA, Formula IB or Formula II or pharmaceutically acceptable salts thereof for the use in the manufacture of a medicament for use in treating subjects in need of such treatment including but not limited to immunodeficient subjects, or pre- or post-organ and/or tissue transplantation subjects.

In some embodiments, the present disclosure provides suspension formulations of the compounds of Formula I, Formula IA, Formula IB or Formula II or pharmaceutically acceptable salts thereof for use in prophylactic treatment or prevention SARS-CoV-2 infection and/or SARS-CoV-2 associated disease and/or disorder. The present disclosure provides suspension formulation of the compounds of Formula I, Formula IA, Formula IB or Formula II or pharmaceutically acceptable salts thereof for use in treating subjects in need of such treatment including but not limited to immunodeficient subjects, or pre- or post-organ and/or tissue transplantation subjects.

The formulations of the present disclosure are used in manufacturing a medicament in prophylactic treatment and/or prevention SARS-CoV-2 infection and/or SARS-CoV-2 associated disease and/or disorder.

In another embodiment, the present disclosure provides compositions (e.g., pharmaceutical compositions) with desirable pharmacokinetic characteristics. For example, the compositions of the disclosure may provide a blood level of the compounds of Formula I, Formula IA, Formula IB or Formula II or pharmaceutically acceptable salts thereof which, after metabolism to the therapeutically-active form (e.g., the diphosphate equivalent), results in blood levels of the metabolite that do not induce toxicity.

### Dosage Regimens

The regimen of administration can affect what constitutes a pharmaceutically effective amount. The compounds of Formula I, Formula IA, Formula IB or Formula II or pharmaceutically acceptable salts thereof can be administered to the subject either prior to or after the onset of a disease. In some embodiments, the compound is administered as a single dose. Further, several divided dosages, as well as staggered dosages can be administered daily or sequentially, or the dose can be continuously infused, or can be a bolus injection. Further, the dosages can be proportionally increased or decreased as indicated by the exigencies of the therapeutic or prophylactic situation. Further, the dosages may be co-administered in combination with another antiviral.

In some embodiments, the compound is administered prior to the onset of a disease. In some embodiments, the compound is administered prior to the onset of infection. In some embodiments, the compound is administered about 24 or about 8 hours before infection. In some embodiments, the compound is administered 24±4, 24±2, 24±1, or 24±0.5 hours before infection. In some embodiments, the compound is administered 8±4, 8±2, 8±1, or 8±0.5 hours before infection. In some embodiments, the compound is administered at about the time of infection. In some embodiments, the compound is administered within 4 hours, within 2 hours, within 1 hour, or within a half hour of infection. In some embodiments, the compound is administered after infection. In some embodiments, the compound is administered about 8 or about 16 hours after infection. In some embodiments, the compound is administered 8±4, 8±2, 8±1, or 8±0.5 hours after before infection. In some embodiments, the compound is administered 16±4, 16±2, 16±1, or 16±0.5 hours after before infection.

In some embodiments, the administration results in a reduced viral titer (e.g. in lung) as compared to a comparable subject without administration. In some embodiments, the administration results in a reduction in viral titer of at least about 4 log 10 copies/mL, at least about 3.9 log 10 copies/mL, at least about 3.8 log 10 copies/mL, at least about 3.7 log 10 copies/mL, at least about 3.6 log 10 copies/mL, at least about 3.5 log 10 copies/mL, or at least about 3.4 log 10 copies/mL, as compared to a comparable subject without administration. In some embodiments, the administration results in a reduction in viral titer of at least about 3 log 10 copies/mL, at least about 2.9 log 10 copies/mL, at least about 2.8 log 10 copies/mL, at least about 2.7 log 10 copies/mL, at least about 2.6 log 10 copies/mL, at least about 2.5 log 10 copies/mL, or at least about 2.4 log 10 copies/mL, as compared to a comparable subject without administration.

In some embodiments, the reduction in viral titer is measured about 1 day, about 2 days, about 3 days, about 4 days, about 5 days, about 7 days, or about 10 days post-infection. In some embodiments, the viral titer is measured by plaque assay. In some embodiments, the viral titer is measured by focus formation assay. In some embodiments, the viral titer is measured by end-point dilution assay (e.g. TCID₅₀, LD₅₀, and/or EID₅₀). In some embodiments, the viral titer is measured by quantitative RNA analysis (e.g. qPCR).

The dosage regimen utilizing the compounds can also be selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter or arrest the progress of the condition.

In some embodiments, the disease or condition to be treated is a coronaviral infection.

In a preferred aspect, the disease or condition to be treated is a SARS-CoV-2 viral infection.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy.

In some embodiments, the dosage is between about 0.001 mg and about 2400 mg. In some embodiments, the dosage is between about 0.1 mg and 2400 mg. In some embodiments, the dosage is between about 5 mg and about 2400 mg. In some embodiments, the dosage is between about 10 mg and about 2400 mg. In some embodiments, the dosage is between about 100 mg and about 2400 mg.

### Routes of Administration

The compounds of the present disclosure, or pharmaceutically acceptable salts, esters or derivatives thereof, can be administered orally, transdermally, subcutaneously, intramuscularly, and intravenously. In some embodiments, the compound is administered orally. One skilled in the art will recognize the advantages of certain routes of administration.

Dosage forms for the topical or transdermal administration of a compound of this disclosure include powders, sprays, ointments, pastes, creams, lotions, gels, solutions, patches and inhalants. In some embodiments, the active compound is mixed under sterile conditions with a pharmaceutically acceptable carrier, and with any preservatives, buffers or propellants that are required.

In some embodiments, the compounds are formulated for pulmonary administration (i.e. administration via the lung). In some embodiments, the pulmonary administration is administration by inhalation. For administration by inhalation, the compounds may be delivered in the form of an aerosol spray from pressured container or dispenser, which contains a suitable propellant, e.g., a gas such as carbon dioxide, or a nebulizer.

In some embodiments, a vial containing a compound as described herein is inserted into an inhaler and/or nebulizer. In some embodiments, a vial containing a compound as described herein is inserted into the bottom of the inhaler and/or nebulizer. In some embodiments, the compound is dispensed as fine aerosols through the mouth.

In some embodiments, the compound is administered in aerosolized form. In some embodiments the compound is administered using a nebulizer or inhaler. In some embodiments, the administration using a nebulizer is daily. In some embodiments, the administration using a nebulizer is twice a day. In some embodiments, the administration using a nebulizer is three times a day. In some embodiments, the administration using a nebulizer is four times a day. In some embodiments, the administration using a nebulizer is six times a day. In some embodiments, the administration using a nebulizer is about every 2 hours, about every 4 hours, about every 6 hours, about every 8 hours, about every 10 hours, or about every 12 hours, about every 24 hours. In some embodiments, the administration using a nebulizer is every 2±1 hours, every 2±0.5 hours, or every 2±0.25 hours. In some embodiments, the administration using a nebulizer is every 4±2 hours, every 4±1 hours, or every 4±0.5 hours. In some embodiments, the administration using a nebulizer is every 6±2 hours, every 6±1 hours, or every 6±0.5 hours. In some embodiments, the administration using a nebulizer is every 8±2 hours, every 8±1 hours, or every 8±0.5 hours. In some embodiments, the administration using a nebulizer is every 10±2 hours, every 10±1 hours, or every 10±0.5 hours. In some embodiments, the administration using a nebulizer is every 12±2 hours, every 12±1 hours, or every 12±0.5 hours. In some embodiments, the administration using a nebulizer is every 24±2 hours, every 24±1 hours, or every 24±0.5 hours.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In some embodiments, the compounds are administered intranasally.

A pharmaceutical composition of the disclosure is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

### Combination Therapy

The present disclosure provides methods of preventing or treating a SARS-CoV-2 viral infection in a subject. The methods comprise administering a subject a therapeutically effective amount of a compound described herein. The compounds may be used in a monotherapy or combination therapy regime.

As used herein, “monotherapy” means or refers to the administration of a single active or therapeutic compound (e.g., a compound of Formula I, Formula IA, Formula IB or Formula II) to a subject in need thereof. Preferably, monotherapy will involve administration of a therapeutically effective amount of an active compound. For example, SARS-CoV-2 monotherapy with one of the compounds of the present disclosure, or a pharmaceutically acceptable salt, prodrug, metabolite, analog or derivative thereof, to a subject in need of treatment of norovirus. Monotherapy may be contrasted with combination therapy, in which a combination of multiple active compounds is administered, preferably with each component of the combination present in a therapeutically effective amount. In some aspects, monotherapy with a compound of the present disclosure, or a pharmaceutically acceptable salt, prodrug, metabolite, polymorph or solvate thereof, is more effective than combination therapy in inducing a desired biological effect.

As used herein, “combination therapy” or “co-therapy” includes the administration of a compound of the present disclosure, or a pharmaceutically acceptable salt, prodrug, metabolite, polymorph or solvate thereof, and at least a second agent as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). “Combination therapy” may be, but generally is not, intended to encompass the administration of two or more of these therapeutic agents as part of separate monotherapy regimens that incidentally and arbitrarily result in the combinations of the present disclosure.

“Combination therapy” is intended to embrace administration of these therapeutic agents in a sequential manner, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical.

“Combination therapy” also embraces the administration of the therapeutic agents as described above in further combination with other biologically active ingredients and non-drug therapies. Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

In some embodiments the present disclosure provides a method of treatment, prevention, or delaying on-set of SARS-CoV-2 infection or SARS-CoV-2 virus infection associated disease or disorder, by oral administration to a subject in need thereof a pharmaceutical composition of a therapeutically effective dose of a compound of Formula I, Formula IA, Formula IB or Formula II or a pharmaceutically acceptable salt thereof in combination with one or more therapeutic agent.

In some embodiments, the therapeutic agent is any agent used to treat COVID-19 or to treat infection with a coronavirus, e.g., SARS-CoV-2 virus. In some embodiments, the additional therapeutic agent is a vaccination for SARS-CoV-2. In some embodiments, the vaccine is an mRNA vaccine. In some embodiments, the vaccine is a viral vector vaccine. In some embodiments, the additional therapeutic agent is selected from the group consisting of: sarilumab, tocilizumab, remdesivir, nirmatrelvir, molnupiravir, paxlovid, favipiravir, zanamivir, laninamivir, peramivir, oseltamivir, camostat, a corticosteroid (e.g., dexamethasone, prednisone, or methylprednisone), an anticoagulation drug (e.g., heparin or enoxaparin), a janus kinase inhibitor (e.g. baricitinib), and immunoglobulin therapy (optionally, IVIG, COVID-19 sera, anti-COVID-19 monoclonal antibodies, or blood transfusions using blood obtained from recovered COVID-19 patients). In some embodiments, intravenous immunoglobulin (IVIG) is a blood product prepared from the serum of human donors, e.g., between 1,000 and 15,000 donors per batch. In particular embodiments, COVID-19 sera is obtained from individuals who have recovered from COVID-19 (e.g., human convalescent sera).

The compounds or compositions provided herein may also be used in combination with other active ingredients. In certain embodiments, the compounds may be administered in combination, or sequentially with another therapeutic agent. Such other therapeutic agents include those known for treatment, prevention, or amelioration of one or more signs or symptoms associated with viral infections. It should be understood that any suitable combination of the compounds provided herein with one or more of the above-mentioned compounds and optionally one or more further pharmacologically active substances are considered to be within the scope of the present disclosure. In another embodiment, the compound provided herein is administered prior to or subsequent to the one or more additional active ingredients. In some embodiments, two or more of the agents disclosed herein are administered serially or in combination. The amount of some enhancers can be selected using methods known in the art to enhance the bioavailability of the agent. Any amount can be used that provides a desired response by some enhancers. The dosages may range, in a non-limiting example, from 0.001 mg to about 3000 mg of compound per kilogram of body weight per day, e.g., 0.01 to 500 mg/kg, or e.g., 0.1-20 mg/kg.

The pharmacokinetic behavior of a composition will vary somewhat from subject to subject within a population. The numbers described above for the compositions disclosed herein are based on the average behavior in a population. The present disclosure is intended to encompass compositions that on average fall within the disclosed ranges, even though it is understood that certain subjects may fall outside of the ranges.

The pharmaceutical compositions can be included in a container, pack, or dispenser together with instructions for administration. The present disclosure provides a kit including, in addition to a pharmaceutical composition of any one of the disclosed compounds, a container, pack, or dispenser together with instructions for administration.

A compound of the present disclosure, or a pharmaceutically acceptable salt, prodrug, metabolite, analog or derivative thereof, may be administered in combination with a second antiviral compound. For example, as noted above, the compositions of the present disclosure may include the compounds as described above in combination with one or more (e.g., 1, 2, 3) additional active agents such as described in this section in analogous manner as known in the art. Additional antiviral active agents that may be used with the compounds of the present disclosure in carrying out the present methods include, but are not limited to, those that target the M2 ion channel in influenza A viruses (e.g., the adamantanes, such as amantadine and rimantadine); those that inhibit viral uncoating following entry into the cell, agents that block release of the newly formed virio4 ns from the surface of infected cells (e.g., the neuraminidase inhibitors, such as oseltamivir and zanamivir).

### SARS-CoV-2

The present disclosure provides a method of treatment, prevention, or delaying on-set of SARS-CoV-2 infections or SARS-CoV-2-infection-associated diseases or disorders by orally administering to a subject a pharmaceutical composition comprising a therapeutically effective dose of a compound of Formula I, Formula IA, Formula IB or Formula II, a pharmaceutically acceptable salt thereof, or a morphic form thereof. In some embodiments, the present disclosure provides a method of treatment, prevention, or delaying on-set of SARS-CoV-2 infections or SARS-CoV-2-infection-associated diseases or disorders by orally administering to a subject a pharmaceutical composition comprising a therapeutically effective dose of a compound of Formula I, Formula IA, Formula IB or Formula II, a pharmaceutically acceptable salt thereof, or a morphic form thereof in combination with one or more of compound or composition selected from an immunosuppressant and/or an antiviral agent.

In some embodiments, the present disclosure provides use of a compound of the present disclosure, a morphic form thereof, or a pharmaceutically acceptable salt thereof in the manufacture of a medicament for treating SARS-CoV-2 as disclosed herein.

In some embodiments, the present disclosure provides use of a compound of the present disclosure, a morphic form thereof, or a pharmaceutically acceptable salt thereof in the manufacture of a medicament for preventing SARS-CoV-2 infection in a subject in need thereof.

In some embodiments, the virus is an RNA virus.

In some embodiments, the RNA virus is a positive-strand RNA virus.

In some embodiments, the positive-strand RNA virus is a coronavirus. In some embodiments, the coronavirus is a betacoronavirus. In some embodiments, the coronavirus is a Sarbecovirus. In some embodiments, the coronavirus is a severe acute respiratory syndrome-related coronavirus.

In some embodiments, the disease or disorder is a coronavirus. In some embodiments, the coronavirus is a betacoronavirus. In some embodiments, the coronavirus is a Sarbecovirus. In some embodiments, the coronavirus is a severe acute respiratory syndrome-related coronavirus.

In some embodiments, the coronavirus is SARS-CoV, MERS-CoV, HCoV-NL63 or SARS-CoV-2.

In some embodiments, the coronavirus is SARS-CoV. In some embodiments, the coronavirus is MERS-CoV. In some embodiments, the coronavirus is HCoV-NL63. In some embodiments, the coronavirus is SARS-CoV-2.

In some embodiments, the positive-strand RNA virus is SARS-CoV-2.

In some embodiments, SARS-CoV-2 is one or more variants of the wild-type. In some embodiments, the SARS-CoV-2 variant is 20I/501Y.V1. In some embodiments, the SARS-CoV-2 variant is 20I/501Y.V1 with a 69/70 deletion In some embodiments, the SARS-CoV-2 variant is 20I/501Y.V1 with a 144Y deletion In some embodiments, the SARS-CoV-2 variant is 20I/501Y.V1 with a N501Y mutation. In some embodiments, the SARS-CoV-2 variant is 20I/501Y.V1 with a A570D mutation. In some embodiments, the 5 SARS-CoV-2 variant is 20I/501Y.V1 with a D614G mutation. In some embodiments, the SARS-CoV-2 variant is 20I/501Y.V1 with a P681H mutation.

In some embodiments, the SARS-CoV-2 variant is 20J/501Y.V3. In some embodiments, the SARS-CoV-2 variant is 20J/501Y.V3 with a E484K mutation. In some embodiments, the SARS-CoV-2 variant is 20J/501Y.V3 with a K417N/T mutation. In some embodiments, the SARS-CoV-2 variant is 20J/501Y.V3 with a N501Y mutation. In some embodiments, the SARS-CoV-2 variant is 20J/501Y.V3 with a D614G mutation.

In some embodiments, the SARS-CoV-2 variant is 20H/501.V2. In some embodiments, the SARS-CoV-2 variant is 20H/501.V2 with a E484K mutation. In some embodiments, the SARS-CoV-2 variant is 20H/501.V2 with a K417N mutation. In some embodiments, the SARS-CoV-2 variant is 20H/501.V2 with a N501Y mutation. In some embodiments, the SARS-CoV-2 variant is 20H/501.V2 with a D614G mutation.

In some embodiments, the subject is a mammal. In some embodiments, the mammal is a human.

In some embodiments, the subject has or is suspected of having a coronavirus. In some embodiments, the subject has been or thought to have been exposed to SARS-CoV-2 and has not yet developed signs or symptoms of SARS-CoV-2 infection (e.g. COVID-19).

In some embodiments, the coronavirus is, for example, the virus that causes COVID-19, SARS, NL63 respiratory disease or MERS. Signs and symptoms of a coronavirus infection can be, for example, fever, cough, and shortness of breath.

Effectiveness of compounds of the disclosure can be determined by industry-accepted assays and/or disease models according to standard practices of elucidating the same as described in the art and are found in the current general knowledge.

In some embodiments, the disease or disorder, or associated sign or symptom is COVID-19, cytokine storm syndrome, CRP, acute respiratory distress syndrome (ARDS), sepsis or septic shock, coagulopathy, respiratory failure, heart failure, dyspnea, or a secondary infection. Each of these has been associated with infection by SARS-CoV-2 and/or with COVID-19. According to particular embodiments, methods disclosed herein may be used to treat, inhibit, or reduce the severity of any of these disorders, e.g., as compared to an untreated subject or a predetermined value, e.g., an average obtained from untreated subjects infected with SARS-CoV-2.

In some embodiments, the methods disclosed herein may also be used to delay the onset of or reduce the severity of any of the diseases, disorders, signs or symptoms disclosed herein. In certain embodiments, the delay of onset is at least one day, at least two days, at least three days, at least four days, at least five days, at least six days, at least seven days, or at least ten days.

In some embodiments, the methods disclosed herein are used to inhibit or prevent the progression of disease associated with SARS-CoV-2 infection (e.g., COVID-19) from a less severe form to a more severe form. As used herein, mild form of the disease or disorder is defined as having systemic symptoms (e.g., fever, pain, fatigue, cough, sore throat) but without evidence of pulmonary involvement; moderate form of the disease or disorder is defined as having evidence of pulmonary involvement (e.g., cough or abnormalities visible on CXR examination or Chest CT examination) but without dyspnea or the need for supplemental oxygen; severe form of the disease or disorder is defined as having pulmonary abnormalities with dyspnea and/or hypoxia requiring supplemental oxygen but not in need of intubation and mechanical ventilation; and critical form of the disease or disorder is defined as requiring admission to intensive care for mechanical ventilation and/or treatment of cardiomyopathy with low ejection fraction. In particular embodiments, methods disclosed herein may be practiced to prevent, inhibit, reduce, or delay the progression of the disease or disorder (e.g., COVID-19) from mild to moderate, severe, or critical; or from moderate to severe or critical; or from severe to critical. In some embodiments, the methods disclosed herein reduce the likelihood of the subject infected with SARS-CoV-2 from dying. Thus, any of the methods disclosed herein may be practiced to reduce morbidity and/or mortality of a subject infected with SARS-CoV-2, e.g., a subject having COVID-19. Also, any of the methods disclosed herein may be practiced to enhance the longevity of a subject infected with SARS-CoV-2, e.g., a subject having COVID-19. In some embodiments, longevity is enhanced as compared to longevity in the absence of treatment with the compounds disclosed herein.

In some embodiments, the methods disclosed herein reduce the likelihood of the subject infected with SARS-CoV-2 from being hospitalized or admitted to an intensive care unit. In some embodiments, the methods disclosed herein reduce the duration of time that the subject infected with SARS-CoV-2 is hospitalized or admitted to an intensive care unit. In some embodiments, the methods disclosed herein reduce the likelihood of the subject infected with SARS-CoV-2 requiring intubation and mechanical ventilation.

As discussed above, methods disclosed herein may be used to alter the response of an infected subject to the infection, e.g., the subject's inflammatory response to the infection. In some embodiments, methods disclosed herein may be practiced to inhibit or reduce the virulence or amount of virus in an infected subject. In addition, methods disclosed herein may be practiced to reduce the duration of time that a subject is infected with SARS-CoV-2, or the duration of time that SARS-CoV-2 is measurably detected in the subject.

In some embodiments, methods disclosed herein comprising administering one or more of the compounds herein to a patient diagnosed with or at risk of developing COVID-19, to inhibit one or more property or characteristic of the virus, such as, e.g., viral replication, viral gene expression or viral shedding by the subject.

In some embodiments of any of the methods disclosed herein related to inhibiting or reducing a value or characteristic, e.g., viral replication, viral shedding, viral gene expression levels, viral protein expression levels, or the amount or severity of a disease, disorder, sign or symptom, the reduction or inhibition is at least 5%, at least 10%, at least 20%, at least 30%, at least 40%, at least 50%, at least 60%, at least 70%, at least 80%, at least 90%, or 100%.

In some embodiments of any of the methods disclosed herein related to a duration of time, the duration of time may be, e.g., about 12 hours, one day, two days, three days, four days, five days, one week, two weeks, three weeks, four weeks, six weeks, eight weeks, three months, four months, six months, one year, or longer; and a reduction in the duration of time may be, e.g., a reduction of at least 5%, at least 10%, at least 20%, at least 30%, at least 40%, at least 50%, at least 60%, at least 70%, at least 80%, at least 90%, or 100%.

In some embodiments, any of the methods disclosed herein further comprising administering to the subject an additional therapeutic agent, e.g., an anti-viral agent. In some embodiments, the therapeutic agent is any agent used to treat COVID-19 or to treat infection with a coronavirus, e.g., SARS-CoV-2 virus. In some embodiments, the additional therapeutic agent is selected from the group consisting of: sarilumab, tocilizumab, remdesivir, nirmatrelvir, molnupiravir, paxlovid, favipiravir, oseltamivir, camostat, a CYP3A4 inhibitor (e.g. ritonavir), a corticosteroid (e.g., dexamethasone, prednisone, or methylprednisone), an anticoagulation drug (e.g., heparin or enoxaparin), a janus kinase inhibitor (e.g. baricitinib), and immunoglobulin therapy (optionally, IVIG, COVID-19 sera, anti-COVID-19 monoclonal antibodies, or blood transfusions using blood obtained from recovered COVID-19 patients). In some embodiments, intravenous immunoglobulin (IVIG) is a blood product prepared from the serum of human donors, e.g., between 1,000 and 15,000 donors per batch. In particular embodiments, COVID-19 sera is obtained from individuals who have recovered from COVID-19 (e.g., human convalescent sera).

### General Assay Methods

Another aspect of the invention relates to compounds for use in methods of inhibiting viral infections, comprising the step of treating a sample or subject suspected of having said inhibition with a composition of the invention.

Within the context of the invention, samples suspected of containing a virus include natural or artificial materials such as living organisms; tissue or cell cultures; biological samples such as samples of biological material (blood, serum, urine, cerebrospinal fluid, tears, sputum, saliva, tissue samples and the like); laboratory samples; food, water or air samples; samples of bioproducts such as cell extracts, particularly recombinant cells that synthesize a desired glycoprotein; and the like Typically, it will be suspected that the sample contains an organism that induces a viral infection, often a pathogenic organism such as a tumor virus. Samples may be contained in any medium including water and organic solvent/water mixtures. Samples include living organisms such as humans and man-made materials such as cell cultures.

If desired, the antivirus activity of a compound of the invention after application of the composition can be observed by any method that includes direct and indirect methods to detect such activity. Quantitative, qualitative and semi-quantitative methods for determining such activity are contemplated. Normally, one of the detection methods described above is applied, however, any other method, such as the observation of the physiological properties of a living organism, is also applicable.

The antiviral activity of a compound of the invention can be measured using standard detection protocols that are known. For example, the antiviral activity of a compound can be measured using the following general protocols.

### Virus, Cells, and Medium Used in Assay

Virus stock of SARS-CoV-2 (USA-WA1/2020) is prepared in various cell lines. Specifically, Vero76 cells or Caco2 cells are used. The medium used for both cell lines is minimum essential medium (MEM) supplemented with 2% FBS and 50 μg/mL gentamicin.

### Test and Control Compounds

Compounds are evaluated alongside sangivamycin as an industry standard.

### Neutral Red Assay in Vero E6 Cells

The antiviral activity of compounds are evaluated based on the ability of the compound to prevent virus from causing viral microscopic cytopathic effect CPE) in mammalian cell culture. Eight dilutions of test compound are evaluated, and the effective antiviral concentration determined by regression analysis. The toxicity of the test compound is determined in parallel. An abbreviated test with four dilutions of compound may be employed to screen large numbers of compounds quickly and at a reduced cost. CPE is determined by microscopic observation of cell culture monolayers as well as uptake of neutral red dye.

Compound dilutions are added to 96-well plates containing 80-100% confluent Vero E6 cells. Cells are incubated with compound for 1-2 hours and then infected with virus at a low MOI to achieve >80% cytopathic effect (CPE) within 5 days of incubation. On day 3 post-infection, CPE is quantitated by neutral red staining. Cells are stained with neutral red for 2 hours, dye is removed, and wells are rinsed with PBS. The incorporated dye was extracted in Sorensen citrate buffer/ethanol (50:50) and absorbance is measured on a spectrophotometer at 540 nm. Compound dilution that reduced virus CPE by 50% (EC50) was calculated along with cytotoxicity in the absence of virus infection (CC50).

### Virus Yield Reduction Assay in Caco-2 Cells

The virus yield reduction assay evaluates the ability of the compound to inhibit virus production in mammalian cell culture. This is a two-step assay where virus is first produced in cultures containing the antiviral substance at varying dilutions, followed later by titration of the samples for virus titer by endpoint dilution in 96-well microplates.

The virus yield reduction (VYR) assay set up is similar to that described for the neutral red assay excepting Caco2 cells were used. On day 3 post-infection, cell culture supernatant from wells was collected and infectious virus determined by running a standard endpoint dilution CCID50 assay. Compound dilution that reduced virus yield by 1 log₁₀ was calculated. After collection of cell culture supernatant from wells on day 3 post-infection, plates are stained as described in the neutral red assay to determine CC50 in Caco2 cells.

Compounds showing antiviral activity in the Caco2 cell VYR assay will demonstrate that the compound inhibited the production of infectious virus. The results for the individual compounds are compared to sangivamycin, a close analog of the compounds of the present disclosure. The sangivamycin does not display selectivity between anti-viral activity and toxicity; whereas, the compounds of the present disclosure are generally non-toxic and active against SARS-CoV-2 making them more selective anti-SARS-CoV-2 agents.

### Methods for Preventing Disease or Disorder Due to Virus Reactivation.

The current disclosure also provides a method of preventing a disease or disorder (e.g. COVID-19) in a subject at risk of SARS-CoV-2 infection reactivation, by orally administering to the subject a pharmaceutical composition of a therapeutically effective dose of a compound of Formula I, Formula IA, Formula IB or Formula II, a pharmaceutically acceptable salt thereof, or a morphic form thereof.

### Effect of Food

In some embodiments, the pharmaceutical composition of the current embodiments, e.g., tablet or suspension, may be provided to a subject when the subject is either fasted or in fed conditions.

In other embodiments, the composition comprising a compound of Formula I, Formula IA, Formula IB or Formula II (or a pharmaceutically acceptable salt thereof) may be provided to a subject in combination with food or subsequent to having food. In some embodiments, a compound of Formula I, Formula IA, Formula IB or Formula II (or a pharmaceutically acceptable salt thereof) may be taken by a subject on an empty stomach.

### Patient Population

In certain embodiments, compounds of Formula I, Formula IA, Formula IB or Formula II (or a pharmaceutically acceptable salt thereof), a composition comprising a compound of Formula I, Formula IA, Formula IB or Formula II, or a combination therapy comprising a composition of Formula I, Formula IA, Formula IB or Formula II is administered to a mammal in need thereof (e.g., a human). In some embodiments, the mammal is suffering from a SARS-CoV-2 infection.

In certain embodiments, a compound of Formula I, Formula IA, Formula IB or Formula II, a composition comprising a compound of Formula I, Formula IA, Formula IB or Formula II, or a combination therapy comprising a compound of Formula I, Formula IA, Formula IB or Formula II is administered to a human at risk for developing a SARS-CoV-2 infection. In certain embodiments, a compound of Formula I, Formula IA, Formula IB or Formula II, a composition comprising a compound of Formula I, Formula IA, Formula IB or Formula II, or a combination therapy comprising a compound of Formula I, Formula IA, Formula IB or Formula II is administered to a human with a SARS-CoV-2 infection.

In some embodiments, a compound of Formula I, Formula IA, Formula IB or Formula II, a composition comprising a compound of Formula I, Formula IA, Formula IB or Formula II, or a combination therapy comprising a compound of Formula I, Formula IA, Formula IB or Formula II is administered to a human infant. In other embodiments, a compound of Formula I, Formula IA, Formula IB or Formula II, or a combination therapy comprising a compound of Formula I, Formula IA, Formula IB or Formula II is administered to a human child. In other embodiments, a compound of Formula I, Formula IA, Formula IB or Formula II, a composition comprising a compound of Formula I, Formula IA, Formula IB or Formula II, or a combination therapy comprising a compound of Formula I, Formula IA, Formula IB or Formula II is administered to a human adult. In yet other embodiments, a compound of Formula I, Formula IA, Formula IB or Formula II, a composition comprising a compound of Formula I, Formula IA, Formula IB or Formula II, or a combination therapy comprising a compound of Formula I, Formula IA, Formula IB or Formula II is administered to an elderly human.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

All patents, patent applications, and publications mentioned herein are hereby incorporated by reference in their entireties. However, where a patent, patent application, or publication containing express definitions is incorporated by reference, those express definitions should be understood to apply to the incorporated patent, patent application, or publication in which they are found, and not to the remainder of the text of this application, in particular the claims of this application.

### EXAMPLES

It is understood that data values described in the examples are approximate, unless indicated otherwise, and that the values are subject to experimental and/or instrumental variations.

### Example 1. In Vitro Antiviral Activity of Compound 1 Against SARS-CoV-2

### Assay Protocol

Virus, cells, and medium used in assay: Virus stock of SARS-CoV-2 (USA-WA1/2020) was prepared in Vero76 cells. Cells used for the assays were Vero E6 and Caco2 cells. The medium used for both cell lines was minimum essential medium (MEM) supplemented with 2% FBS and 50 μg/mL gentamicin.

Test and control compounds: Compound 1 was evaluated alongside sangivamycin as an industry standard.

Neutral red assay in Vero E6 cells: Compound dilutions were added to 96-well plates containing 80-100% confluent Vero E6 cells. Cells were incubated with compound for 1-2 hours and then infected with virus at a low MOI to achieve >80% cytopathic effect (CPE) within 5 days of incubation. On day 3 post-infection, CPE was quantitated by neutral red staining. Briefly cells were stained with neutral red for 2 hours, dye removed, and wells rinsed with PBS. The incorporated dye was extracted in Sorensen citrate buffer/ethanol (50:50) and absorbance measured on a spectrophotometer at 540 nm. Compound dilution that reduced virus CPE by 50% (EC50) was calculated along with cytotoxicity in the absence of virus infection (CC50).

Virus yield reduction assay in Caco-2 cells: The virus yield reduction (VYR) assay set up was similar to that described for the neutral red assay excepting Caco2 cells were used. On day 3 post-infection, cell culture supernatant from wells was collected and infectious virus determined by running a standard endpoint dilution CCID50 assay. Compound dilution that reduced virus yield by 1 log₁₀ was calculated. After collection of cell culture supernatant from wells on day 3 post-infection, plates were stained as described in the neutral red assay to determine CC50 in Caco2 cells.

### Results

The observed in vitro antiviral activity against SARS-CoV-2 are summarized in Table 2A below.

TABLE 2A

| | Neutral Red in Vero | | | Virus Yield Reduction | | |
|---|---|---|---|---|---|---|
| | E6 cells | | | in Caco2 cells | | |
| | EC50 | CC50 | | EC90 | CC50 | |
| | (μM) | (μM) | SI | (μM) | (μM) | SI90 |
| Compound 1 | 56 | >100 | >1.8 | 2.1 | >100 | >47 |
| Sangivamycin | 0.007 | 0.01 | 1.9 | 0.12 | 0.12 | 1.0 |

EC50: 50% effective antiviral concentration
CC50: 50% cytotoxic concentration of compound without virus added
EC90: Calculated concentration to reduce virus yield by 1 log (90%)
SI: Selectivity Index (CC50/EC50)

Compound 1 showed antiviral activity in the Caco2 cell VYR assay demonstrating that the compound inhibited the production of infectious virus. Inhibition in the neutral red CPE assay in Vero E6 cells was observed but the potency was less than in the VYR assay. Similar differences between results in the two assay formats have been reported for another SARS-CoV-2 antiviral, remdesivir and could be attributed to assay readout. The reported in vitro activity for remdesivir against SARS-CoV-2 in the different assay formats tested spans between 0.77 μM and 23 μM which is similar to that seen for Compound 1. In comparison sangivamycin, a close analog of Compound 1, did not display substantial selectivity between activity and toxicity in either assay.

### Example 2: Compound 1 Inhibits SARS-CoV-2 in Human Airway Epithelial Cell Cultures and Exhibits Prophylactic and Therapeutic Efficacy Against Respiratory Disease in a Mouse Model of SARS-CoV-2 Infection

Compound 1 inhibits SARS-CoV-2 replication in primary human airway epithelial cell cultures (average EC50=0.9 μM). Compound 1 is not genotoxic or mitotoxic, has a favorable toxicology profile (rats/dogs, oral GLP), and was generally well-tolerated up to 2400 mg in a healthy volunteer Phase 1 study of oral Compound 1. In vivo evaluation of aerosol Compound 1 was performed in uninfected BALB/c mice (tolerability/PK) or mice infected intranasally with 10,000 PFU of SARS-CoV-2-MA10 (a mouse-adapted SARS-CoV-2 virus) (N=79). Prophylactic administration of Compound 1 (q8 hours) starting 24 hours prior to infection reduced average viral titers in lung on day 4 post-infection by 3.62 log 10 and prevented weight loss/clinical progression versus placebo (n=9/group). The most comprehensive single study randomized mice to Compound 1 at −8, 0, ±8 or ±16 hours post-infection or placebo (n=6/group). Compound 1 treatment significantly reduced lung viral RNA (Kruskal-Wallis p<0.0001) and viral titer (p<0.0001) at day 4 post-infection relative to placebo. Compound 1 treatment post-infection also protected mice from clinical signs of disease, significantly reducing weight loss and decreasing lung pathology compared to placebo (p<0.0001). For example, Compound 1 initiated 16 hours post-infection reduced average viral lung titer by 2.56 log 10, prevented weight loss and reduced average clinical score from 3 to 0 (day 4). Overall, no gross safety or tolerability issues were identified in Compound 1 treated mice. These data indicate that aerosol Compound 1 is an effective prophylactic and therapeutic for SARS-CoV-2 in a preclinical animal model and support further development as an antiviral against emerging coronaviruses.

### Example 3: Evaluation of Antiviral Activity of Compound 1 Against SARS-CoV-2 and Related Viruses In Vitro

The antiviral efficacy of Compound 1 against SARS-CoV-2, murine hepatitis virus, and bat-derived SARS-like coronaviruses was studied in vitro.

Murine hepatitis virus (MHV) strain MHV-A59 and SARS-CoV-2 strain USA-WA1 2020 were used. In each case, the viruses were modified to encode a nanoluciferase (nLuc) reporter. For MHV, the majority of the coding sequence of orf4a and 4b were replaced with nLuc. The SARS-CoV-2 nLuc virus has been described previously (Hou Y J, Okuda K, Edwards C E, et al. SARS-CoV-2 Reverse Genetics Reveals a Variable Infection Gradient in the Respiratory Tract. Cell. 2020. 182:429-446).

Compound 1 was tested against MHV, SARS-CoV-2, and bat-derived SARS-like coronaviruses in cell culture. The MHV assay was performed in DBT cells. DBT cells are mouse brain tumor cells transformed by Rous sarcoma virus which support propagation of MHV. Cells were infected at a multiplicity of infection (MOI) of 0.1 infectious units per cell as determined by TCID₅₀ assay. The drugs were added to cells at the indicated concentrations 1 h prior to infection and luciferase activity was measured at 10 h post infection by using the Promega Nano-Glo Luciferase Assay System.

The SARS-CoV-2 assay was performed in primary human epithelial (HAE) cells grown at the air-liquid interface in transwell plates. Washed HAE cells on transwells were transferred into a fresh plate containing the appropriate drug concentrations in each well. The cells were infected with SARS-CoV-2 at an MOI of 1 just after placing them in drug-containing medium. Luciferase activity was measured at 24 h post infection as above. The same assay was used for the bat-derived SARS-like coronaviruses

For each assay, percent inhibition was calculated by comparing luciferase activity in cells treated with compound to that in cells treated with vehicle (DMSO) control. Cells were treated with 2.5 μM remdesivir as a control where indicated. Cytotoxicity was measured in uninfected cells in parallel assays using the Sigma TOX7 assay for LDH release.

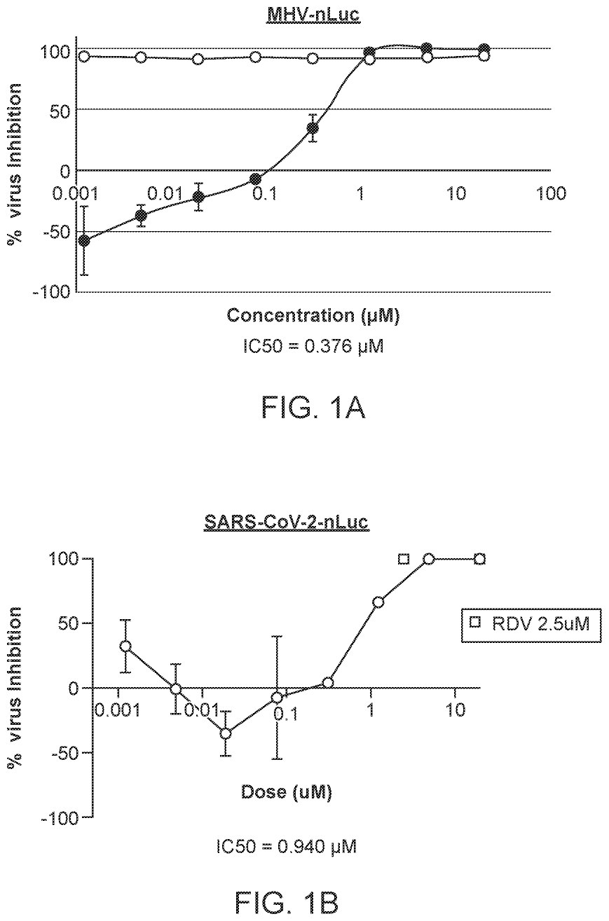

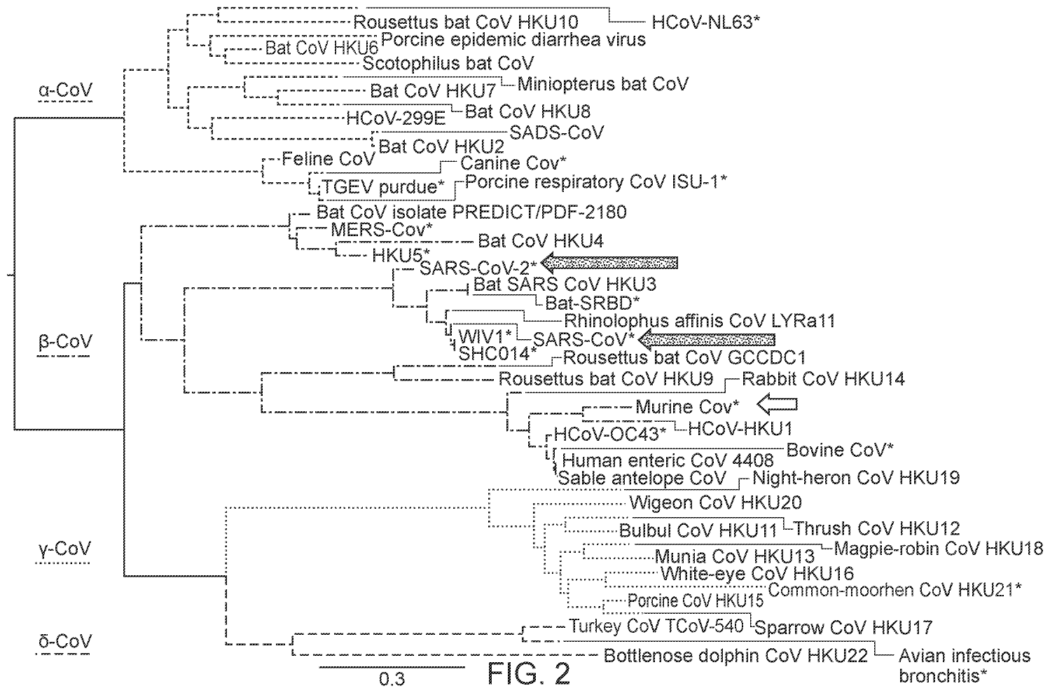

Compound 1 was active against MHV and SARS-CoV-2 with EC₅₀ values of 0.38 μM and 0.54 μM (mean), respectively (Table 2B). FIGS. 1A and 1B show representative dose-response curves. No effect on cell viability was seen at any concentration (not shown). Compound 1 was also active against two strains of bat-derived SARS-like viruses, WIV1 and SHC014. The EC₅₀ values against these viruses were in the range of 1 μM. A phylogenetic tree illustrating the relationship between these viruses and SARS is shown in FIG. 2.

TABLE 2B
Compound 1 EC50 Values Against nLuc SARS-CoV-2, nLuc
MHV, and Bat-Derived SARS-Like Coronaviruses.

| Virus | EC50 | Notes |
|---|---|---|
| SARS-CoV-2 | 0.16 | HAE cells donor code yellow;* 5 point dose- |
| | | response curve |
| | 0.55 | HAE cells donor code blue; 5 point dose- |
| | | response curve |
| | 0.38 | HAE cells donor code purple; 5 point dose- |
| | | response curve |
| SARS-CoV-2 | 0.35 | HAE cells donor code yellow; 7 point dose- |
| | | response curve |
| SARS-CoV-2 | 0.31 | HAE cells donor code yellow; 4 point dose- |
| | | response curve |
| SARS-CoV-2 | 1.1 | HAE cells donor code yellow; 8 point dose- |
| | | response curve |
| SARS-CoV-2 | 0.94 | HAE cells donor code yellow; 8 point dose- |
| | | response curve |
| Mean | 0.54 | |
| SARS-CoV-2 | | |
| MHV | 0.38 | DBT cells; 8 point dose-response curve |
| WIV1 | 1.01 | HAE cells |
| SHC014 | 0.98 | HAE cells |

*HAE cells derived from different donors are indicated by different color codes

Previously, Compound 1 was tested against SARS-CoV-1 and showed mixed results, with EC50 values of >100 and 37 μM (see, e.g. U.S. Pat. No. 9,708,359). These tests were cytopathic effect (CPE) assays performed in Vero cells (Vero E6 or Vero 76) with neutral red (vital stain-based) or CellTiter Glo (ATP luminescence-based) readouts. Following these studies, initial assays using SAR-CoV-2 compared activity of Compound 1 in Vero E6 cells using a neutral red CPE readout and in Caco2 cells using a viral yield reduction (endpoint dilution CCID₅₀ assay as readout). The Compound 1 EC₅₀ in Vero 76 cells was 56 μM and the 90% viral yield reduction in Caco-2 cells was 2.1 μM. Without wishing to be bound by theory, these results suggest activity may vary by cell line.

Without wishing to be bound by theory, the lower antiviral activity seen in the Vero cell assays may reflect a relatively lower degree of phosphorylation to Compound 60 (triphosphate form of Compound 1) in those cells. In the current example, good antiviral activity for Compound 1 was observed in primary human airway epithelial cells, a relevant target cell for SARS-CoV-2 in patients.

### Example 4: Evaluation of Compound 1 in the hACE2 Mouse Model

The human ACE2 transgenic mouse model of SARS-CoV-2 virus disease (Rathnasinghe, Raveen et al. “Comparison of transgenic and adenovirus hACE2 mouse models for SARS-CoV-2 infection.” Emerging microbes & infections vol. 9,1 (2020): 2433-2445.) was used to evaluated efficacy of Compound 1 for activity against virus infection.

While the model does not recapitulate normal human disease, it shows viremia in lungs beyond day 2 and animals succumb to disease on day 7-9. Pneumonia is present but may be the product of encephalitis resulting in breathing problems as viremia in the brain is also high when they succumb. However, for evaluation of small molecule treatments, a drop in the virus load in the lung is likely sufficient to indicate efficacy. A drop of 10-fold or more would represent a significant change in virus load and potential for treatment.

The dose of Compound 1 was at 50 mg/kg dosed once per day by the intraperitoneal route. The compound was formulated in 3% DMSO, 4.8% PEG400 in 5% Dextrose. Groups of 10 mice, (5 female and 5 male) were first challenged by intranasal installation of 10⁴ PFU (plaque forming units) of SARS-CoV-2. Treatment was started 4-6 h later and repeated once each day for 4 additional days. On Day 5, animals were euthanized and viremia measured in the lungs by qPCR. Primers were those used by the CDC detection assay and were obtained from IDT. Each sample was assayed twice. Cq (quantification cycle) values were determined

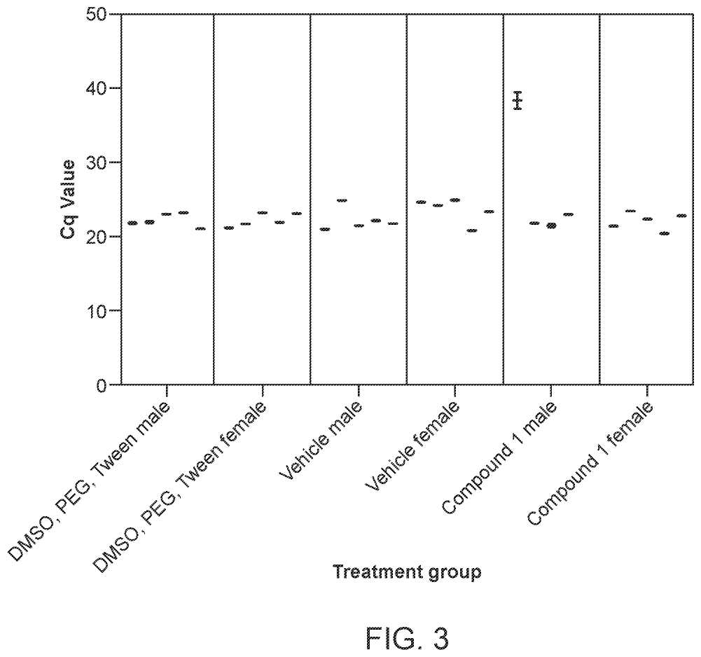

The results are shown in FIG. 3. Treatment groups are indicated. Each set of points represent a sample from a single mouse assayed twice. Virus genome loads were detected by qPCR and fell within the log-linear portion of the dose response curve for the assay. For Compound 1, treatment did not alter the virus genome levels detected in lungs, which gave an average Cq of 22. One of the male mice showed lower than expected virus genome loads (Cq of 38). Without wishing to be bound by theory, this is likely due to failure of virus challenge or recovery of RNA. Vehicle alone showed a slightly higher but statistically insignificantly different Cq of 23. Another vehicle control that contained Tween 20 was evaluated and gave an identical outcome.

This test indicates that Compound 1 was not effective at inhibiting SARS-CoV-2 in this animal disease model and for the formulation and dosage tested. Other doses, formulations or frequency of dosing may give a different outcome.

### Example 5: Evaluation of Clinical and Antiviral Efficacy of Prophylaxis Vs Treatment with Inhaled Aerosolized Compound 1 in a SARS-CoV-2-MA10 Mouse Model

The clinical and antiviral efficacy of aerosol Compound 1 delivered three-times (TID) daily when dosing was initiated 8 hours pre-infection versus 24 hours post-infection to mice infected with SARS-CoV-2-MA10.

The experiment utilized a mouse pie cage comprised of 12 separate wedges open to a central core from which aerosol enters through tubing from a nebulizer compressor containing a liquid formulation of Compound 1. Each pie wedge holds a single mouse and aerosolized liquid enters each wedge through 3 mm holes (n=5) at the core and exits through one 3 mm hole in the center of the outer edge.

For each treatment group, 9 female, 9-11 week old BALB/c mice were placed in 9 individual wedges of the inhalation device described above. The input holes of the remaining 3 wedges were taped off to maximize delivery to the 9 mice. A liquid formulation of 5 mg/mL Compound 1 (pH 6.0) or vehicle control was prepared immediately prior to dosing and dispensed into the compressor chamber. Group 1 (n=9) received vehicle control. For Group 2 (n=9), Compound 1 dosing was initiated 8 hours prior to infection. Compound 1 was initiated in Group 3 (n=9) 24 hours post-infection. The entire contents of the formulation solutions (9 mL) were aerosolized and delivered to the 9 mice over a period of 20-30 min.

In Compound 1 treated animals, this method delivered 5 mg Compound 1 to each wedge containing a single mouse over the period of nebulization. The dose of Compound 1 inhaled by each mouse is currently unknown, could not be greater than 5 mg but is believed to be significantly lower.

Mice were dosed as described above either 8 hours prior to- or 24 hours after-infection with 10,000 PFU SARS-CoV-2-MA10 via nasal instillation. After initiation of dosing, Compound 1 (or vehicle) were administered every 8 hours for 4 days post-infection. Body weights and detailed clinical observations were recorded once daily prior to dosing. Clinical observations were recorded according to the rubric presented in Table 3.

TABLE 3
Detailed clinical observations

| Clinical Score | Clinical observations |
|---|---|
| 0 | Normal |
| 1 | Unkempt coat |
| 2 | Unkempt coat and hunched posture |
| 3 | Unkempt coat, hunched posture and reduced |
| | movement |
| 4 | Minimal spontaneous movement +/− dyspnea |
| | (humane endpoint) |
| 5 | Moribund, dead or euthanized |

On Day 4, mice were euthanized via an overdose of isoflurane anesthetic 8 hours after the final dosing occasion. A portion of the inferior lung lobe was homogenized in 70:30 methanol:water and frozen at −80° C. for future bioanalysis of Compound 1 and Compound 60 concentrations. Half the nasal tissue and the superior and middle lung lobes (combined) were each isolated and homogenized in cell culture media for viral titer analysis. The remaining nasal tissue and postcaval lung lobe were each homogenized in Trizol and prepared for nsp4 qPCR. The left lung lobe was fixed in formalin for future histopathological analysis. Upon collection of the lungs, gross lung discoloration was noted and scored according to the rubric presented in Table 4.

TABLE 4
Gross lung discoloration (GLD)

| | | Lobe surface |
|---|---|---|
| GLD Score | Discoloration Grade* | area affected |
| 0.0 | Normal, Pink | N/A |
| 0.5 | Mild/moderate OR Severe | <33% OR <5% |
| 1.0 | Severe | 5-33% |
| 1.5 | Mild/moderate | 33-67% |
| 2.0 | Severe | 33-67% |
| 2.5 | Mild/moderate | 67-99% |
| 3.0 | Severe | 67-99% |
| 3.5 | Mild/moderate | 100% |
| 4.0 | Severe | 100% |

*Mild/moderate discoloration = dark pink or purple; Severe discoloration = red, maroon, or dark brown.
NOTE:
If lungs have a combination of mild/moderate and severe discoloration, whichever severity level is predominant will determine the overall GLD score.

Relative nsp4 expression in the postcaval lobe was calculated by normalizing duplicate nsp4 Cq values from each animal to the duplicate GAPDH Cq values in the same animal. The average normalized nsp4 expression was calculated for the vehicle group and the relative nsp4 expression was determined for each animal (including vehicle treated animals) compared to the vehicle group average.

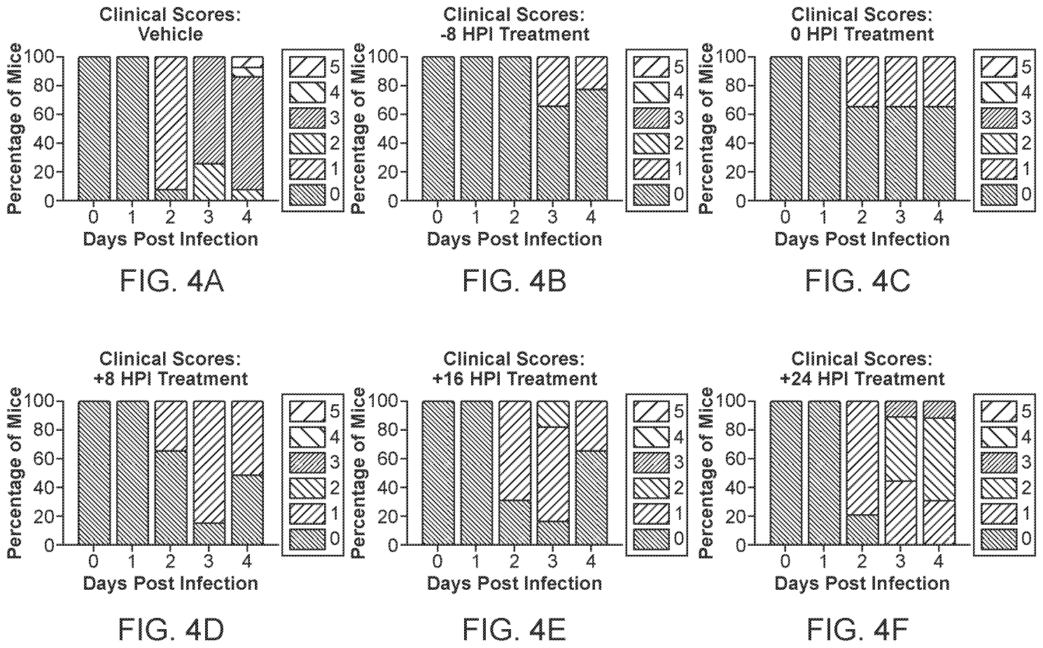

Infected animals treated with vehicle control exhibited clinical signs of infection beginning on day 2 with further deterioration of condition through days 3 and 4 resulting in mortality of 1/9 animals on day 4 (FIG. 4A). Initiation of treatment 8 hours prior to infection protected animals from clinical signs of disease (FIG. 4B) and initiation 24 hours post-infection mitigated the clinical signs of disease relative to placebo but did not provide the protection of Compound 1 initiated prior to infection (FIG. 4F).

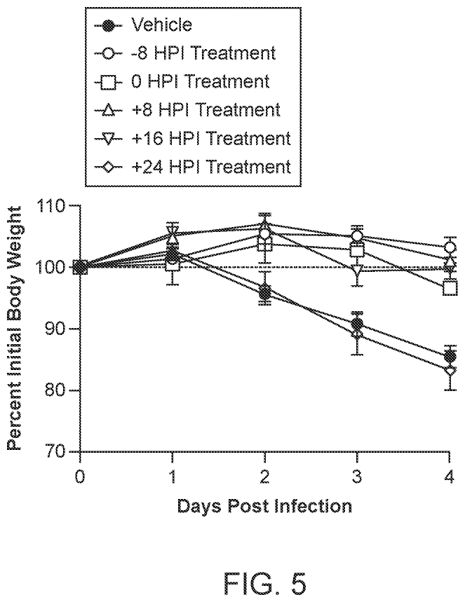

Body weights of animals given vehicle or Compound 1 initiated 24 hours post infection decreased by approximately 15% baseline body weight by day 4 post infection. Animals treated with Compound 1 initiated 8 hours prior to infection maintained or slightly gained weight during the 4 day post-infection period (FIG. 5).

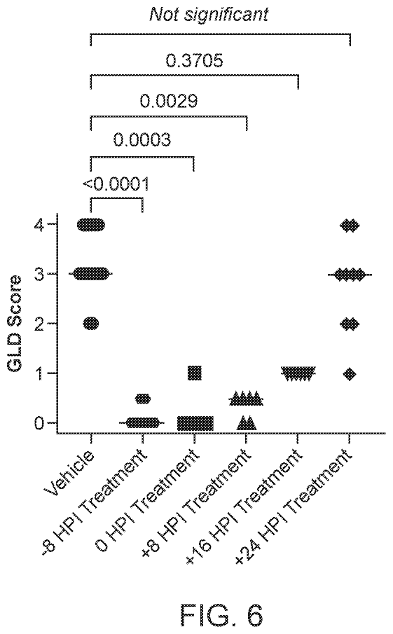

Lungs of infected mice administered vehicle exhibited severe discoloration on >67% of the surface area of the lung lobe. Administration of Compound 1 initiated 24 hours post-infection mitigated the discoloration relative to vehicle treated mice, but the discoloration was severe in at least 5% of the lung lobe in 1 animal, and greater than 33% of the surface area of the lobes in the remaining 8 animals. Only a slight area of mild/moderate discoloration was observed in the lung lobes of animals administered Compound 1 initiating 8 hours prior to infection (FIG. 6).

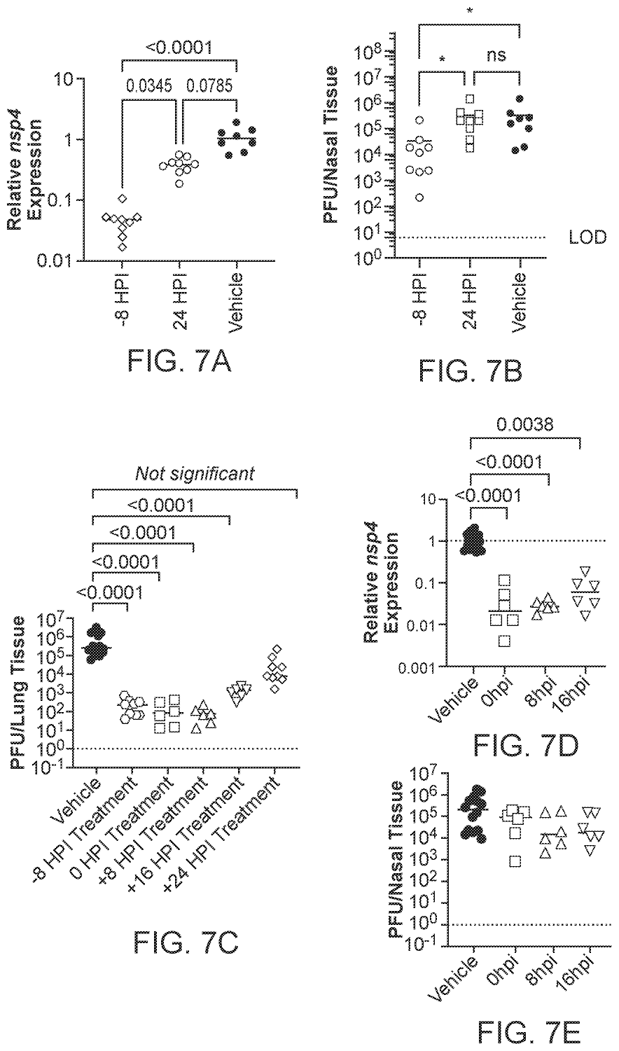

The delivery of 5 mg Compound 1 via inhalation exposure (inhaled deposited dose not determined) administered TID initiating 8 hours prior to infection with SARS-CoV-2-MA10 significantly reduced viral RNA (nsp4) (FIG. 7A) and viral lung and nasal titer (FIGS. 7B and 7C) compared to vehicle. Compound 1 administration 24 hours after infection reduced lung RNA and lung titer levels relative to vehicle treated animals but did not reach significance (FIGS. 7A-7C).

### Example 6: Evaluation of Clinical and Antiviral Efficacy of Inhaled Aerosol Compound 1 Initiated at 0-, 8- and 16-Hours Post-Infection in a SARS-CoV-2-MA10 Mouse Model

The clinical and antiviral efficacy of aerosol Compound 1 delivered three-times daily (TID) when initiated at the time of infection versus 8- and 16-hours post-infection to mice infected with SARS-CoV-2-MA10 was evaluated.

The experiment utilized a mouse pie cage comprised of 12 separate wedges open to a central core from which aerosol enters through tubing from a nebulizer compressor containing a liquid formulation of Compound 1. Each pie wedge holds a single mouse and aerosolized liquid enters each wedge through 3 mm holes (n=5) at the core and exits through one 3 mm hole in the center of the outer edge.

For the initiation of treatment, 6-12 female, 9-11 week old BALB/c mice were placed in individual wedges of the inhalation device described above. The input holes of the remaining 3 wedges were taped off to deliver ˜1 mL aerosolized Compound 1 (5 mg) to each wedge. A liquid formulation of 5 mg/mL Compound 1 (pH 6.0) or vehicle control was prepared immediately prior to dosing and dispensed into the compressor chamber. Group 1 (n=6) received vehicle control. For Group 2 (n=6), Compound 1 dosing was initiated immediately before infection. Compound 1 was initiated in Group 3 (n=6) 8 hours post-infection and Group 4 (n=6) initiated treatment 16 hours post-infection. The entire contents of the formulation solutions (9 mL) were aerosolized and delivered to the 6 mice over a period of 20-30 min.

In Compound 1 treated animals, this method delivered 5 mg Compound 1 to each wedge containing a single mouse over the period of nebulization. The dose of Compound 1 inhaled by each mouse is currently unknown, could not be greater than 5 mg but is believed to be significantly lower.

Mice were dosed as described above either 0-, 8- or 16-hours after infection with 10,000 PFU SARS-CoV-2-MA10 via nasal instillation. After initiation of dosing, Compound 1 (or vehicle) were administered every 8 hours for 4 days post-infection. Body weights and detailed clinical observations were recorded once daily prior to dosing. Clinical observations were recorded according to the rubric presented above in Table 3.

On Day 4, mice were euthanized via an overdose of isoflurane anesthetic 8 hours after the final dosing occasion. A portion of the inferior lung lobe was homogenized in 70:30 methanol:water and frozen at −80° C. for future bioanalysis of Compound 1 and Compound 60 concentrations. Half the nasal tissue and the superior and middle lung lobes (combined) were each isolated and homogenized in cell culture media for viral titer analysis. The remaining nasal tissue and postcaval lung lobe were each homogenized in Trizol and prepared for nsp4 RT-PCR. The left lung lobe was fixed in formalin for future histopathological analysis. Upon collection of the lungs, gross lung discoloration was noted and scored according to the rubric presented above in Table 4.

Relative nsp4 expression in the postcaval lobe was calculated by normalizing duplicate nsp4 Cq values from each animal to the duplicate GAPDH Cq values in the same animal. The average normalized nsp4 expression was calculated for the vehicle group and the relative nsp4 expression was determined for each animal (including vehicle treated animals) compared to the vehicle group average.

Infected animals treated with vehicle control exhibited clinical signs of infection beginning on day 2 with further deterioration of condition through days 3 and 4 resulting in mortality of 1/9 animals on day 4 (FIG. 4A). Initiation of treatment at the time of infection and 8 hours post-infection protected animals from clinical signs of disease (FIGS. 4C and 4D) and initiation 16 hours post-infection mitigated the clinical signs of disease relative to placebo and 2 animals which deteriorated on day 3 improved condition by day 4 (FIG. 4E).

Body weights of animals given vehicle decreased by approximately 15% of baseline body weight by day 4 post infection. Animals treated with Compound 1 initiated at the time of infection or 8- or 16-hours post-infection maintained or slightly gained weight during the 4 day post-infection period (FIG. 5).

Lungs of infected mice administered vehicle exhibited severe discoloration on >67% of the surface area of the lung lobe. Administration of Compound 1 initiated at the time of infection or 8 or 16 hours post-infection mitigated the discoloration relative to vehicle treated mice. Only a slight area of mild/moderate discoloration was observed in the lung lobes of animals administered Compound 1 initiating at all 3 timepoints (FIG. 6B).

The delivery of 5 mg Compound 1 via inhalation exposure (inhaled deposited dose not determined) administered TID initiating at the time of infection or 8- or 16-hours post-infection with SARS-CoV-2-MA10 significantly reduced lung viral RNA (nsp4) (FIG. 7D) and viral lung titer (FIG. 7E) compared to vehicle. Compared to vehicle, Compound 1 treatment initiated at the time of infection or 8- or 16-hours post-infection reduced lung viral titer 2.18, 1.93 and 3.1 logs, respectively. Nasal titer levels were lower than in vehicle treated animals but did not reach significance (FIG. 7C).

### Example 7: Evaluation of Antiviral Efficacy of Prophylactic Dosing of Inhaled Aerosolized Compound 1 at 16 Hours Post-Infection in a SARS-CoV-2-MA10 Mouse Model

The tolerability and antiviral efficacy of aerosol Compound 1 was studied when delivered three-times daily when initiated 8-hours pre-infection to mice infected with SARS-CoV-2-MA10.

The experiment utilized a mouse pie cage comprised of 12 separate wedges open to a central core from which aerosol enters through tubing from a nebulizer compressor containing a liquid formulation of Compound 1. Each pie wedge contains a single mouse and aerosolized liquid enters each wedge through 3 mm holes (n=5) at the core and exits through one 3 mm hole in the center of the outer edge.

Nine female, 9-11 week old BALB/c mice were housed in 9 individual wedges of the inhalation device described above for each dosing occasion. The input holes of the remaining 3 wedges were taped off to maximize delivery to the 9 mice. A liquid formulation of 5 mg/mL Compound 1 (pH 6.0) was prepared immediately prior to dosing and dispensed into the compressor chamber. The entire contents (9 mL) of the formulation solution were aerosolized and delivered to the 9 mice over a period of 20-30 min. This method delivered 5 mg Compound 1 to each wedge containing a single mouse over the period of nebulization. The dose of Compound 1 inhaled by each mouse is currently unknown, could not be greater than 5 mg, but is believed to be significantly lower.

The procedure described above was repeated with an additional 10 mice administered vehicle formulation via aerosol in the pie chamber.

Mice were dosed as described above 8 hours prior to infection with 10,000 PFU SARS-CoV-2-MA10 via nasal instillation. A second aerosolized dose was administered immediately before infection and a third dose was administered 8 hours after infection. Mice were euthanized 24 hours after the first dose of Compound 1 (16 hours post-infection) via an overdose of isoflurane anesthetic. A portion of the inferior lung lobe was homogenized in 70:30 methanol:water and frozen at −80° C. for future bioanalysis of Compound 1 and Compound 60 concentrations. Half the nasal tissue and the superior and middle lung lobes (combined) were each isolated and homogenized in cell culture media for viral titer analysis. The remaining nasal tissue and postcaval lung lobe were each homogenized in Trizol and prepared for nsp4 qPCR. The left lung lobe was fixed in formalin for future histopathological analysis.

Relative nsp4 expression in the postcaval lobe was calculated by normalizing duplicate nsp4 Cq values from each animal to the duplicate GAPDH Cq values in the same animal. The average normalized nsp4 expression was calculated for the vehicle group and the relative nsp4 expression was determined for each animal (including vehicle treated animals) compared to the vehicle group average.

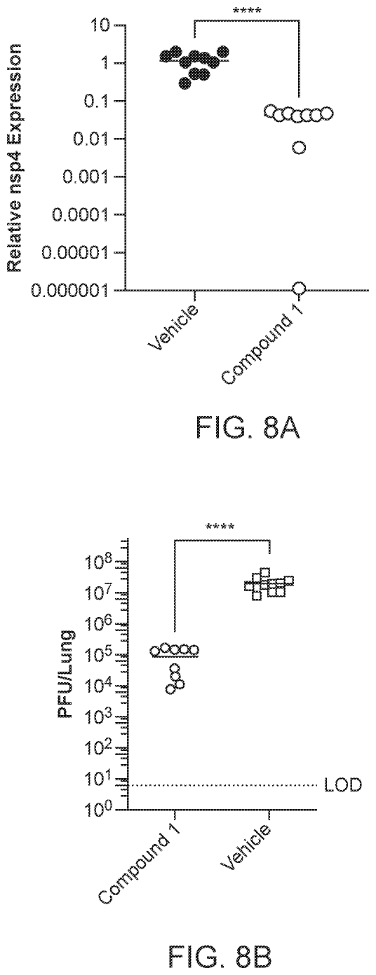

The delivery of 5 mg Compound 1 via inhalation exposure (inhaled deposited dose not determined) administered TID initiating 8 hours prior to infection with SARS-CoV-2-MA10 significantly reduced viral RNA (nsp4) (FIG. 8A) and viral titer in lung (FIG. 8B; Table 5) compared to vehicle. Nasal RNA and titer levels were not significantly changed compared to vehicle control.

TABLE 5
Individual animal viral (superior + middle lobe) lung and nasal titer

| Treatment | Animal ID | Lung Titer | Nasal Titer |
|---|---|---|---|
| Compound 1 | 1 | 1.31E+05 | 1.74E+04 |
| (5 mg/kg; TID) | 2 | 1.35E+05 | 3.36E+03 |
| | 3 | 7.50E+03 | 2.33E+05 |
| | 4 | 2.01E+04 | 3.15E+05 |
| | 5 | 1.41E+05 | 3.56E+04 |
| | 6 | 1.39E+05 | 3.09E+04 |
| | 7 | 1.61E+05 | 9.50E+02 |
| | 8 | 1.06E+04 | 2.99E+05 |
| | 9 | 3.66E+04 | 2.16E+05 |
| Vehicle | 10 | 1.91E+07 | 2.54E+04 |
| | 11 | 2.29E+07 | 2.59E+04 |
| | 12 | 1.68E+07 | 2.66E+05 |
| | 13 | 2.94E+07 | 2.71E+05 |
| | 14 | 4.43E+07 | 2.11E+04 |
| | 15 | 1.03E+07 | 1.38E+05 |
| | 16 | 1.06E+07 | 1.46E+05 |
| | 17 | 2.00E+07 | 2.01E+05 |
| | 18 | 1.83E+07 | 2.81E+05 |
| | 19 | 8.00E+06 | 3.61E+04 |

### Example 8: Healthy (Uninfected) BALB/c Mouse Pharmacokinetic Study with Compound 1 Inhalation Delivery

Tolerability and pharmacokinetics (PK) of a single dose of Compound 1 delivered as aerosol via nebulized liquid in a flow-past pie-chamber model to healthy BALB/c mice. Compound 1 and Compound 60 concentrations were evaluated in plasma and lung tissue were determined.

The experiment utilized a mouse pie cage comprised of 12 separate wedges open to a central core from which aerosol enters through tubing from a nebulizer compressor containing a liquid formulation of Compound 1. Each pie wedge holds a single mouse and aerosolized liquid enters each wedge through 3 mm holes (n=5) at the core and exits through one 3 mm hole in the center of the outer edge.

Nine female, 9-11 week old BALB/c mice were housed in 9 individual wedges of the inhalation device described above. The input holes of the remaining 3 wedges were taped off to maximize delivery to the 9 mice. A liquid formulation of 5 mg/mL Compound 1 (pH 6.0) was prepared immediately prior to dosing and dispensed into the compressor chamber. The entire contents (9 mL) of the formulation solution were aerosolized and delivered to the 9 mice over a period of 20-30 min. This method delivered 5 mg Compound 1 to each wedge containing a single mouse over the period of nebulization. The dose of Compound 1 inhaled by each mouse is currently unknown, could not be greater than 5 mg but is believed to be significantly lower.

Mice were monitored for adverse clinical signs with special attention given to signs related to eye irritation.

One-hour post-dose, 3 mice were euthanized via an overdose of isoflurane anesthetic and blood was collected via cardiocentesis, processed to plasma and frozen for bioanalysis. Additionally, the left, middle, post-caval, superior and inferior lung lobes were collected, immediately homogenized in 70:30 methanol:water and frozen at −80° C. for bioanalysis of Compound 1 and Compound 60. This euthanization and collection process was repeated with 3 mice at 3 hours post-dose and with the last 3 mice at 8 hours post-dose.

Plasma was analyzed for Compound 1 by liquid chromatography/tandem mass spectrometry (LC-MS/MS) using a qualified assay where the dynamic range of the assay was 0.500-500 ng/mL Compound 1.

Lung homogenate extracts were analyzed for Compound 1 and Compound 60 by LC-MS/MS in positive ionization mode under optimized conditions for detection of Compound 60 positive ions formed by electrospray ionization. The dynamic ranges of the assay were 0.100-100 ng/mL for Compound 1 and 0.100-500 ng/mL for Compound 60.

No clinical signs of irritation or intolerability were observed during the dosing period or during the 8-hour post-dose period prior to termination.

The delivery of 5 mg Compound 1 via inhalation exposure (inhaled dose not determined) delivered low levels of Compound 1 to the systemic circulation (Table 6). It is unknown whether systemic concentrations were achieved via the lung or through the oral route via self-cleaning. Compound 1 was detectable in plasma through 8 hours post-dose.

While significant variability was observed, the delivery of 5 mg Compound 1 via inhalation exposure (inhaled dose not determined) resulted in the presence of detectable levels of both Compound 1 and Compound 60 through 8 hours post-dose (Tables 7 and 8).

Compound 1 concentrations were approximately 20-fold higher in the lung than the plasma at 1 and 3 hours post-dose and approximately 12-fold higher at 8 hours post-dose. Without wishing to be bound by theory, higher concentrations of Compound 60 vs Compound 1 in the lung suggests that Compound 1 associated with lung tissue is absorbed and efficiently converted to Compound 60, the presumptive active antiviral. Molar conversion of Compound 60 concentrations per gram of tissue yielded average intracellular concentrations of 5 μM, 1 hours post-dose and 3 μM, 3 hours post-dose. At 8 hours post-dose, the average measured Compound 60 concentration across lobes was 0.5 μM.

TABLE 6
Plasma Compound 1 concentrations

| | Compound 1 | Time | Compound 1 | Mean Compound |
|---|---|---|---|---|
| Animal | Delivered Dose | Post-Dose | Concentration | 1 Concentration |
| # | (mg) | (hr) | (ng/mL) | (ng/mL) |
| 1 | 5 | 1 | 32.5 | 41.77 |
| 2 | 5 | 1 | 39.1 | |
| 3 | 5 | 1 | 53.7 | |
| 4 | 5 | 3 | 18.5 | 18.03 |
| 5 | 5 | 3 | 20.8 | |
| 6 | 5 | 3 | 14.8 | |
| 7 | 5 | 8 | 11.9 | 13.3 |
| 8 | 5 | 8 | 15.4 | |
| 9 | 5 | 8 | 12.6 | |

TABLE 7
Lung tissue Compound 1 concentrations

| Time post- | | Lung Lobe Concentrations (pmol/g) | | | | |
|---|---|---|---|---|---|---|
| Dose (hr) | Animal ID | Inferior | Left | Middle | PC | Superior |
| 1 | 1 | 2436 | 1521 | 1323 | 986 | 1867 |
| | 2 | 1084 | 769 | 988 | 673 | 1219 |
| | 3 | 374 | 378 | 1061 | 430 | 1212 |
| 3 | 4 | 110 | 140 | 129 | 264 | 305 |
| | 5 | 209 | 106 | 601 | 1515 | 398 |
| | 6 | 93 | 116 | 205 | 581 | 167 |
| 8 | 7 | 38 | 43 | 73 | 50 | 37 |
| | 8 | 323 | 169 | 137 | 1010 | 270 |
| | 9 | 51 | 60 | 99 | 57 | 65 |

TABLE 8
Lung tissue Compound 60 concentrations

| Time post- | | Lung Lobe Concentrations (pmol/g) | | | | |
|---|---|---|---|---|---|---|
| Dose (hr) | Animal ID | Inferior | Left | Middle | PC | Superior |
| 1 | 1 | 3767 | 6512 | 7373 | 7212 | 7347 |
| | 2 | 4461 | 3883 | 3634 | 4140 | 4058 |
| | 3 | 4159 | 2482 | 7319 | 4575 | 9849 |
| 3 | 4 | 1359 | 1482 | 1078 | 1513 | 2439 |
| | 5 | 3281 | 981 | 6530 | 14316 | 4384 |
| | 6 | 1138 | 1550 | 731 | 2942 | 2080 |
| 8 | 7 | 444 | 552 | 640 | 697 | 389 |
| | 8 | 421 | 383 | 348 | 617 | 814 |
| | 9 | 402 | 407 | 563 | 463 | 720 |

### EQUIVALENTS

The disclosure can be embodied in other specific forms without departing from the spirit or essential characteristics thereof. The foregoing embodiments are therefore to be considered in all respects illustrative rather than limiting on the disclosure described herein. Scope of the disclosure is thus indicated by the appended claims rather than by the foregoing description, and all changes that come within the meaning and range of equivalency of the claims are intended to be embraced therein.

What is claimed is:

1. A method of treating or preventing a SARS-CoV-2 infection or a SARS-CoV-2-infection associated disease or disorder (e.g., COVID-19) comprising administering, or causing to be administered, to a subject in need thereof an effective amount of a compound of Formula II, 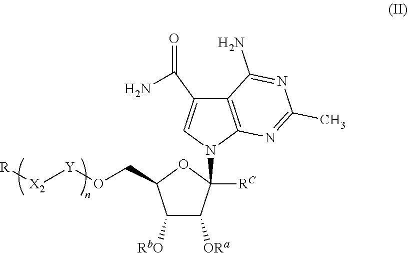
a prodrug thereof, a metabolite thereof, or a pharmaceutically acceptable salt, solvate, enantiomer, diastereomer, racemate or mixture thereof, wherein:
Y is —C(O)—, or 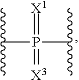
wherein X¹ is independently O, NH, or S, X² is independently a bond, —O—, —S—, or —NH—, and X³ is independently —OR, —NHR, or —SR;
R is independently —H, —C₁-C₂₀alkyl, —C₂-C₂₀alkenyl, —C₂-C₂₀alkynyl, —C₃-C₈cycloalkyl, —C₄-C₈cycloalkenyl, aryl, heteroaryl, or heterocyclyl, wherein each alkyl, alkenyl, alkynyl, cycloalkyl, cycloalkenyl, aryl, heteroaryl, or heterocyclyl is optionally substituted with one or more halogen, oxo, R¹, —OR¹, —NR¹R², —SR¹, —OC(O)R¹, —C(O)OR¹, —NHC(O)OR¹, or —NHC(O)R¹;
R^{a} and R^{b} are each independently, at each occurrence, —H, —C₁-C₂₀alkyl, —C₂-C₂₀alkenyl, —C₂-C₂₀alkynyl, —C₃-C₈cycloalkyl, —C₄-C₈cycloalkenyl, aryl, heteroaryl, or heterocyclyl, wherein each alkyl, alkenyl, alkynyl, cycloalkyl, cycloalkenyl, aryl, heteroaryl, or heterocyclyl is optionally substituted with one or more halogen, oxo —OR¹, —NR¹R², —SR¹, —OC(O)R¹, —C(O)OR¹—NHC(O)OR¹, or —NHC(O)R¹;
R¹ and R² are each independently, at each occurrence, —H, —C₁-C₂₀alkyl, —C₂-C₂₀alkenyl, —C₂-C₂₀alkynyl, —C₃-C₈cycloalkyl, —C₄-C₈cycloalkenyl, aryl, heteroaryl, or heterocyclyl, wherein each alkyl, alkenyl, alkynyl, cycloalkyl, cycloalkenyl, aryl, heteroaryl, or heterocyclyl is optionally substituted with one or more halogen, oxo, R³, R⁴, —OR³, —NR³R₄, —SR³, —OC(O)R³, —C(O)OR³, —NHC(O)OR³, or —NHC(O)R³;
R³ and R⁴ are each independently, at each occurrence, —H, —C₁-C₂₀alkyl, —C₂-C₂₀alkenyl, —C₂-C₂₀alkynyl, —C₃-C₈cycloalkyl, —C₄-C₈cycloalkenyl, aryl, heteroaryl, or heterocyclyl, wherein each alkyl, alkenyl, alkynyl, cycloalkyl, cycloalkenyl, aryl, heteroaryl, or heterocyclyl is optionally substituted with one or more halogen, oxo, aryl, heteroaryl, —OH, —NH₂, —SH, —OC(O)H, —C(O)OH, —NHC(O)OH, or —NHC(O)H;
R^{c} is independently —H or -D; and
n is independently 0, 1, 2 or 3.

2. The method of claim 1, wherein the compound is: 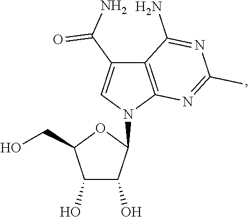 [SMILES: Cc1nc(N)c2c(C(N)=O)cn([C@@H]3O[C@H](CO)[C@@H](O)[C@H]3O)c2n1]
a prodrug thereof, or a metabolite thereof.

3. The method of claim 1, wherein the compound is:

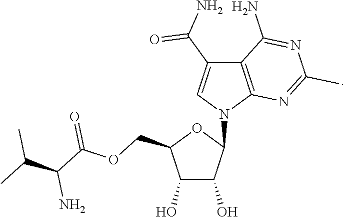 [SMILES: Cc1nc(N)c2c(C(N)=O)cn([C@@H]3O[C@H](COC(=O)[C@@H](N)C(C)C)[C@@H](O)[C@H]3O)c2n1]

4. The method of claim 1, wherein the compound is:

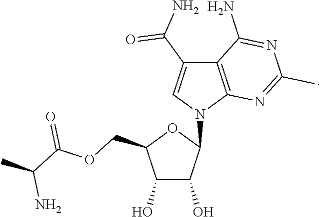 [SMILES: Cc1nc(N)c2c(C(N)=O)cn([C@@H]3O[C@H](COC(=O)[C@H](C)N)[C@@H](O)[C@H]3O)c2n1]

5. The method of claim 1, wherein the compound is:

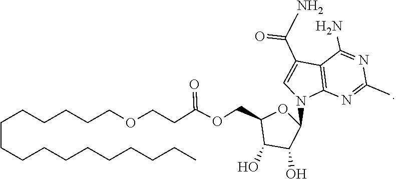 [SMILES: CCCCCCCCCCCCCCCCOCCC(=O)OC[C@H]1O[C@@H](n2cc(C(N)=O)c3c(N)nc(C)nc32)[C@H](O)[C@@H]1O]

6. The method of claim 1, wherein the compound is:

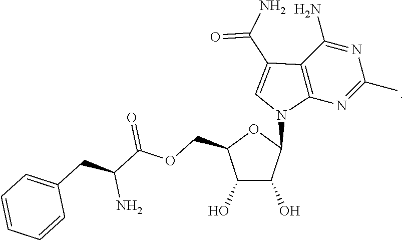 [SMILES: Cc1nc(N)c2c(C(N)=O)cn([C@@H]3O[C@H](COC(=O)[C@@H](N)Cc4ccccc4)[C@@H](O)[C@H]3O)c2n1]

7. The method of claim 1, wherein the compound is:

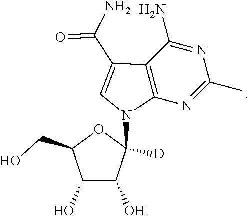 [SMILES: [2H][C@@]1(n2cc(C(N)=O)c3c(N)nc(C)nc32)O[C@H](CO)[C@@H](O)[C@H]1O]

8. The method of claim 1, wherein the compound is:

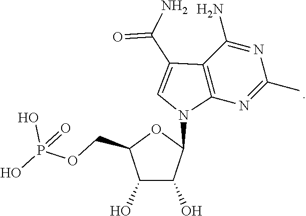 [SMILES: Cc1nc(N)c2c(C(N)=O)cn([C@@H]3O[C@H](COP(=O)(O)O)[C@@H](O)[C@H]3O)c2n1]

9. The method of claim 1, wherein the compound is:

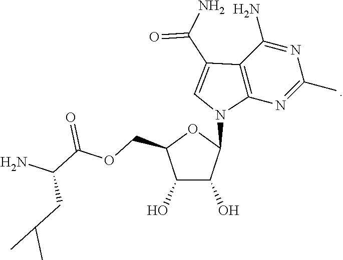 [SMILES: Cc1nc(N)c2c(C(N)=O)cn([C@@H]3O[C@H](COC(=O)[C@@H](N)CC(C)C)[C@@H](O)[C@H]3O)c2n1]

10. The method of claim 1, wherein the compound is:

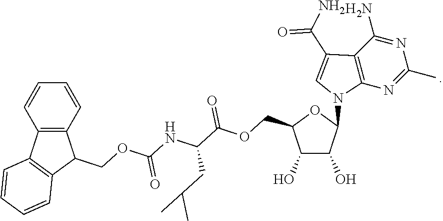 [SMILES: Cc1nc(N)c2c(C(N)=O)cn([C@@H]3O[C@H](COC(=O)[C@H](CC(C)C)NC(=O)OCC4c5ccccc5-c5ccccc54)[C@@H](O)[C@H]3O)c2n1]

11. The method of claim 1, wherein the compound is:

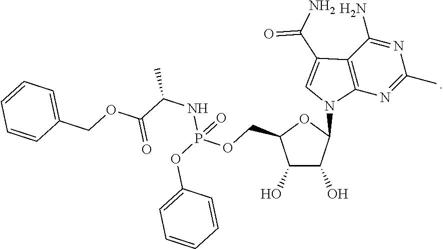 [SMILES: Cc1nc(N)c2c(C(N)=O)cn([C@@H]3O[C@H](COP(=O)(N[C@@H](C)C(=O)OCc4ccccc4)Oc4ccccc4)[C@@H](O)[C@H]3O)c2n1]

12. The method of claim 1, wherein the compound is:

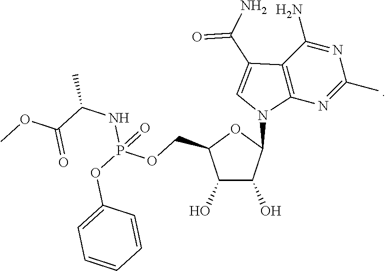

13. The method of claim 1, wherein the compound is:

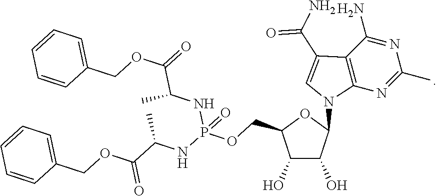 [SMILES: Cc1nc(N)c2c(C(N)=O)cn([C@@H]3O[C@H](COP(=O)(N[C@@H](C)C(=O)OCc4ccccc4)N[C@H](C)C(=O)OCc4ccccc4)[C@@H](O)[C@H]3O)c2n1]

14. The method of claim 1, wherein the compound is:

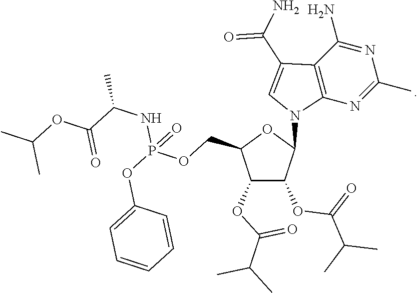 [SMILES: Cc1nc(N)c2c(C(N)=O)cn([C@@H]3O[C@H](COP(=O)(N[C@@H](C)C(=O)OC(C)C)Oc4ccccc4)[C@@H](OC(=O)C(C)C)[C@H]3OC(=O)C(C)C)c2n1]

15. The method of claim 1, wherein the compound is:

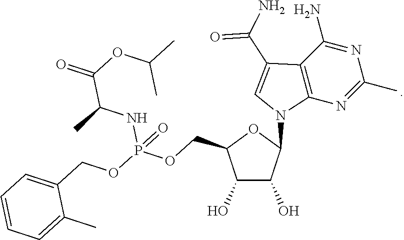 [SMILES: Cc1nc(N)c2c(C(N)=O)cn([C@@H]3O[C@H](COP(=O)(N[C@@H](C)C(=O)OC(C)C)OCc4ccccc4C)[C@@H](O)[C@H]3O)c2n1]

16. The method of claim 1, wherein the compound is:

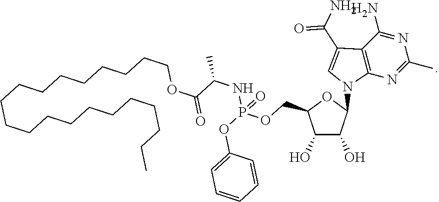 [SMILES: CCCCCCCCCCCCCCCCCCCCOC(=O)[C@H](C)NP(=O)(OC[C@H]1O[C@@H](n2cc(C(N)=O)c3c(N)nc(C)nc32)[C@H](O)[C@@H]1O)Oc1ccccc1]

17. The method of claim 1, wherein the compound is:

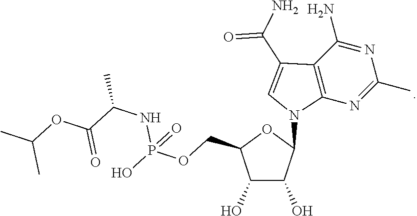 [SMILES: Cc1nc(N)c2c(C(N)=O)cn([C@@H]3O[C@H](COP(=O)(O)N[C@@H](C)C(=O)OC(C)C)[C@@H](O)[C@H]3O)c2n1]

18. The method of claim 1, wherein the compound is:

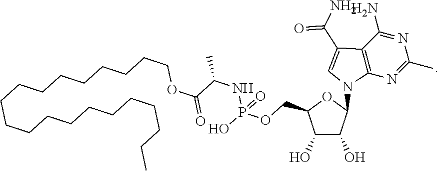 [SMILES: CCCCCCCCCCCCCCCCCCCCOC(=O)[C@H](C)NP(=O)(O)OC[C@H]1O[C@@H](n2cc(C(N)=O)c3c(N)nc(C)nc32)[C@H](O)[C@@H]1O]

19. The method of claim 1, wherein the compound is:

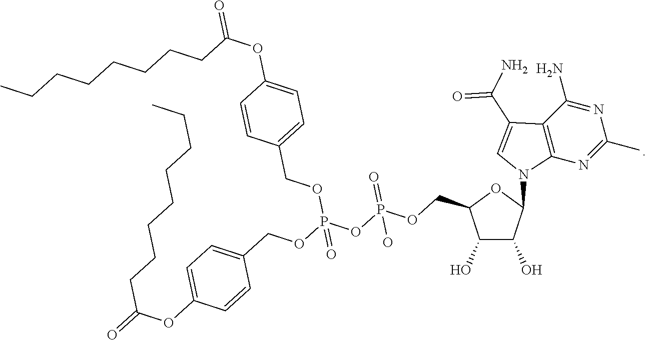 [SMILES: CCCCCCCCC(=O)Oc1ccc(COP(=O)(OCc2ccc(OC(=O)CCCCCCCC)cc2)OP(=O)(O)OC[C@H]2O[C@@H](n3cc(C(N)=O)c4c(N)nc(C)nc43)[C@H](O)[C@@H]2O)cc1]

20. The method of any one of claims 1-19, wherein the compound of Formula II is administered to the lungs of the subject.

21. The method of any one of claims 1-20, wherein the compound of Formula II is administered to the subject via inhalation.

22. The method of any one of claims 1-20, wherein the compound of Formula II is administered to the subject intratracheally, nasally, or intranasally.

23. The method of any one of claims 1-22, wherein the compound of Formula II is administered to the subject via a nebulizer, metered dose inhaler (MDI), a propellant-driven metered dose inhaler (pMDI) breath actuated devices (BAIs) such as a dry powder inhaler (DPI), heat vaporizer, soft mist inhaler, thermal aerosol inhaler, or electrohydrodynamic-based solution misting inhaler.

24. The method of any one of claims 1-22, wherein the compound of Formula II is administered to the subject via a dry powder inhaler.

25. The method of any one of claims 1-22, wherein the compound of Formula II is administered to the subject via a nebulizer.

26. The method of any one of claims 1-22, wherein the compound of Formula II is administered to the subject via a metered dose inhaler.

27. The method of any one of claims 1-26, wherein the method comprises administering a pharmaceutical composition comprising a compound of Formula II and a pharmaceutically acceptable carrier.

28. The method of claim 27, wherein the pharmaceutical composition is formulated for delivery by inhalation.

29. The method of claim 27, wherein the pharmaceutical composition is formulated for delivery intratracheally, nasally, or intranasally.

30. The method of claim 27, wherein the pharmaceutical composition is formulated for delivery by a nebulizer, metered dose inhaler (MDI), a propellant-driven metered dose inhaler (pMDI) breath actuated devices (BAIs) such as a dry powder inhaler (DPI), heat vaporizer, soft mist inhaler, thermal aerosol inhaler, or electrohydrodynamic-based solution misting inhaler.

31. The method of claim 27, wherein the pharmaceutical composition is formulated for delivery by a dry powder inhaler.

32. The method of claim 27, wherein the pharmaceutical composition is formulated for delivery by a nebulizer.

33. The method of claim 27, wherein the pharmaceutical composition is formulated for delivery by metered dose inhaler.

34. The method of claims 1-33, wherein the method further comprises administering a second therapeutic agent.

35. The method of claim 34, wherein the second therapeutic agent is selected from the group consisting of: an mRNA vaccine, a viral vector vaccine, sarilumab, tocilizumab, remdesivir, nirmatrelvir, molnupiravir, paxlovid, favipiravir, oseltamivir, camostat, a CYP3A4 inhibitor (e.g. ritonavir), a corticosteroid, an anticoagulation drug, a janus kinase inhibitor (e.g. baricitinib), and immunoglobulin therapy, intravenous immunoglobulin (IVIG), COVID-19 sera, anti-COVID-19 monoclonal antibodies, or blood transfusions from recovered COVID-19 patients.

36. A method of any one of the preceding claims, wherein the method prevents a patient from developing acute respiratory distress syndrome.

37. A method of any one of the preceding claims, wherein the method prevents the progression of acute respiratory distress syndrome in a patient.

38. Use of a compound of Formula II for preparing a medicament for preventing a SARS-CoV-2 infection or a SARS-CoV-2-infection associated disease or disorder (e.g., COVID-19) in a patient in need thereof.

39. A compound of Formula II for use in preventing a SARS-CoV-2 infection or a SARS-CoV-2-infection associated disease or disorder (e.g., COVID-19) in a patient in need thereof.

40. A method for treating or preventing a SARS-CoV-2 infection or a SARS-CoV-2-infection associated disease or disorder (e.g., COVID-19) in a patient in need thereof, comprising administering, or causing to be administered, to the patient a compound of Formula II, a prodrug thereof, a metabolite thereof, or a pharmaceutically acceptable salt, solvate, enantiomer, diastereomer, racemate or mixture thereof.

41. The method of claim 40, comprising causing to be administered to the patient one or more active metabolites by in vivo formation.

42. The method of claim 41, wherein the active metabolite is one or more of a monophosphate, a diphosphate, or a triphosphate analog of a compound of Formula II.

43. The method of claim 42, wherein the active metabolite is Compound 1 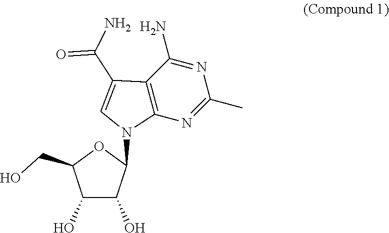 [SMILES: Cc1nc(N)c2c(C(N)=O)cn([C@@H]3O[C@H](CO)[C@@H](O)[C@H]3O)c2n1]
or a monophosphate, a diphosphate, or a triphosphate analog thereof.
